SEQUENCE LISTING (293 sequences)

SEQ ID NO: 1 - protein, 4 aa, synthetic construct
AQAQ

SEQ ID NO: 2 - protein, 11 aa, synthetic construct
DGTLAVPFKAQ

SEQ ID NO: 3 - protein, 11 aa, synthetic construct
AQTLAVPFSNP

SEQ ID NO: 4 - protein, 11 aa, synthetic construct
DGTLSQPFRAQ

SEQ ID NO: 5 - protein, 11 aa, synthetic construct
DGTADKPFRAQ

SEQ ID NO: 6 - protein, 11 aa, synthetic construct
AQFVVGQSYAQ

SEQ ID NO: 7 - protein, 11 aa, synthetic construct
AQRYQGDSVAQ

SEQ ID NO: 8 - protein, 11 aa, synthetic construct
AQWSTNAGYAQ

SEQ ID NO: 9 - protein, 11 aa, synthetic construct
AQERVGFAQAQ

SEQ ID NO: 10 - protein, 11 aa, synthetic construct
AQWMTHGSAAQ

SEQ ID NO: 11 - protein, 11 aa, synthetic construct
AQWPTSYDAAQ

SEQ ID NO: 12 - protein, 11 aa, synthetic construct
DGSSSYYDAAQ

SEQ ID NO: 13 - protein, 11 aa, synthetic construct
AQGENPGRWAQ

SEQ ID NO: 14 - protein, 11 aa, synthetic construct
DGTGQVTGWAQ

SEQ ID NO: 15 - protein, 11 aa, synthetic construct
DGTGSTTGWAQ

SEQ ID NO: 16 - protein, 7 aa, synthetic construct
NGSGQNQ

SEQ ID NO: 17 - protein, 7 aa, synthetic construct
DGAATKN

SEQ ID NO: 18 - protein, 7 aa, synthetic construct
DGQSSKS

SEQ ID NO: 19 - protein, 11 aa, synthetic construct
  VARIANT 1..3: note = Xaa = any amino acid
XXXYQGDSVAQ

SEQ ID NO: 20 - protein, 11 aa, synthetic construct
  VARIANT 3..5: note = Xaa = any amino acid
AQXXXGDSVAQ

SEQ ID NO: 21 - protein, 11 aa, synthetic construct
  VARIANT 5..7: note = Xaa = any amino acid
AQRYXXXSVAQ

SEQ ID NO: 22 - protein, 11 aa, synthetic construct
  VARIANT 7..9: note = Xaa = any amino acid
AQRYQGXXXAQ

SEQ ID NO: 23 - protein, 11 aa, synthetic construct
  VARIANT 9..11: note = Xaa = any amino acid
AQRYQGDSXXX

SEQ ID NO: 24 - protein, 11 aa, synthetic construct
  VARIANT 1..3: note = Xaa = any amino acid
XXXSTNAGYAQ

SEQ ID NO: 25 - protein, 11 aa, synthetic construct
  VARIANT 3..5: note = Xaa = any amino acid
AQXXXNAGYAQ

SEQ ID NO: 26 - protein, 11 aa, synthetic construct
  VARIANT 5..7: note = Xaa = any amino acid
AQWSXXXGYAQ

SEQ ID NO: 27 - protein, 11 aa, synthetic construct
  VARIANT 7..9: note = Xaa = any amino acid
AQWSTNXXXAQ

SEQ ID NO: 28 - protein, 11 aa, synthetic construct
  VARIANT 9..11: note = Xaa = any amino acid
AQWSTNAGXXX

SEQ ID NO: 29 - protein, 11 aa, synthetic construct
  VARIANT 1..3: note = Xaa = any amino acid
XXXRVGFAQAQ

SEQ ID NO: 30 - protein, 11 aa, synthetic construct
  VARIANT 3..5: note = Xaa = any amino acid
AQXXXGFAQAQ

SEQ ID NO: 31 - protein, 11 aa, synthetic construct
  VARIANT 5..7: note = Xaa = any amino acid
AQERXXXAQAQ

SEQ ID NO: 32 - protein, 11 aa, synthetic construct
  VARIANT 7..9: note = Xaa = any amino acid
AQERVGXXXAQ

SEQ ID NO: 33 - protein, 11 aa, synthetic construct
  VARIANT 9..11: note = Xaa = any amino acid
AQERVGFAXXX

SEQ ID NO: 34 - protein, 11 aa, synthetic construct
  VARIANT 1..3: note = Xaa = any amino acid
XXXMTHGSAAQ

SEQ ID NO: 35 - protein, 11 aa, synthetic construct
  VARIANT 3..5: note = Xaa = any amino acid
AQXXXHGSAAQ

SEQ ID NO: 36 - protein, 11 aa, synthetic construct
  VARIANT 5..7: note = Xaa = any amino acid
AQWMXXXSAAQ

SEQ ID NO: 37 - protein, 11 aa, synthetic construct
  VARIANT 7..9: note = Xaa = any amino acid
AQWMTHXXXAQ

SEQ ID NO: 38 - protein, 11 aa, synthetic construct
  VARIANT 9..11: note = Xaa = any amino acid
AQWMTHGSXXX

SEQ ID NO: 39 - protein, 11 aa, synthetic construct
AQWSTKSGYAQ

SEQ ID NO: 40 - protein, 11 aa, synthetic construct
AQWSTKTGYAQ

SEQ ID NO: 41 - protein, 11 aa, synthetic construct
AQWSTATGYAQ

SEQ ID NO: 42 - protein, 11 aa, synthetic construct
AQWTKNAGYAQ

SEQ ID NO: 43 - protein, 11 aa, synthetic construct
AQWSKNAGYVQ

SEQ ID NO: 44 - protein, 11 aa, synthetic construct
AQWSTHAGYAQ

SEQ ID NO: 45 - protein, 11 aa, synthetic construct
AQWRTHAGYAQ

SEQ ID NO: 46 - protein, 11 aa, synthetic construct
AQWSKNAGYTQ

SEQ ID NO: 47 - protein, 11 aa, synthetic construct
ERWMTHGSAAQ

SEQ ID NO: 48 - protein, 11 aa, synthetic construct
APRYKGDSVAQ

SEQ ID NO: 49 - protein, 11 aa, synthetic construct
AQRYQGDSVQR

SEQ ID NO: 50 - protein, 11 aa, synthetic construct
AQLRVGFAQAQ

SEQ ID NO: 51 - protein, 11 aa, synthetic construct
SQWSTNAGYSQ

SEQ ID NO: 52 - protein, 11 aa, synthetic construct
AQWSTHTGYAQ

SEQ ID NO: 53 - protein, 11 aa, synthetic construct
AQWSTNAGYTQ

SEQ ID NO: 54 - protein, 11 aa, synthetic construct
AQWMTHGSAAM

SEQ ID NO: 55 - protein, 11 aa, synthetic construct
AQWSTKAGYAQ

SEQ ID NO: 56 - protein, 11 aa, synthetic construct
SQRYQGDSVAQ

SEQ ID NO: 57 - protein, 11 aa, synthetic construct
SPRYKGDSVAQ

SEQ ID NO: 58 - protein, 11 aa, synthetic construct
NRSMTHGSAAQ

SEQ ID NO: 59 - protein, 8 aa, synthetic construct
AQAQAQTG

SEQ ID NO: 60 - protein, 14 aa, synthetic construct
AQTLVPFKAQAQTG

SEQ ID NO: 61 - protein, 15 aa, synthetic construct
DGTLAVPFKAQAQTG

SEQ ID NO: 62 - protein, 15 aa, synthetic construct
DGTLSQPFRAQAQTG

SEQ ID NO: 63 - protein, 15 aa, synthetic construct
AQRYQGDSVAQAQTG

SEQ ID NO: 64 - protein, 15 aa, synthetic construct
AQWSTNAGYAQAQTG

SEQ ID NO: 65 - protein, 15 aa, synthetic construct
AQWPTSYDAAQAQTG

SEQ ID NO: 66 - protein, 15 aa, synthetic construct
DGSSSYYDAAQAQTG

SEQ ID NO: 67 - protein, 18 aa, synthetic construct
VHKKLTSSKEDSKDKFAV

SEQ ID NO: 68 - protein, 24 aa, synthetic construct
VHEKEKGTSRNVKEAQDPEDEIAV

SEQ ID NO: 69 - protein, 24 aa, synthetic construct
VHEKEKGTSRNVKEAQDPEDEIAV

SEQ ID NO: 70 - protein, 15 aa, synthetic construct
AKPTPLLGLLQAQTG

SEQ ID NO: 71 - protein, 15 aa, synthetic construct
AKPTPLLLLLQAQTG

SEQ ID NO: 72 - protein, 15 aa, synthetic construct
AQPPLGGLLAQAQTG

SEQ ID NO: 73 - protein, 15 aa, synthetic construct
AKPPGPWAEAQAQTG

SEQ ID NO: 74 - protein, 15 aa, synthetic construct
AQPPLLGGLAQAQTG

SEQ ID NO: 75 - protein, 15 aa, synthetic construct
AQPPGPWAAAQAQTG

SEQ ID NO: 76 - protein, 15 aa, synthetic construct
AQPPGPYAVAQAQTG

SEQ ID NO: 77 - protein, 15 aa, synthetic construct
ASSETATWATQAQTG

SEQ ID NO: 78 - protein, 15 aa, synthetic construct
AQPPGPWAVAQAQTG

SEQ ID NO: 79 - protein, 15 aa, synthetic construct
AQAGGDYDAVQAQTG

SEQ ID NO: 80 - protein, 15 aa, synthetic construct
AQGASGIDQAQAQTG

SEQ ID NO: 81 - protein, 15 aa, synthetic construct
AKPPSPWAEAQAQTG

SEQ ID NO: 82 - protein, 15 aa, synthetic construct
AQGGGEYEEVQAQTG

SEQ ID NO: 83 - protein, 15 aa, synthetic construct
AQAKGLYEAVQAQTG

SEQ ID NO: 84 - protein, 15 aa, synthetic construct
AQPPGPWAEAQAQTG

SEQ ID NO: 85 - protein, 15 aa, synthetic construct
AKPTPLLKLLQAQTG

SEQ ID NO: 86 - protein, 15 aa, synthetic construct
AQAGGDYEAVQAQTG

SEQ ID NO: 87 - protein, 15 aa, synthetic construct
AQPPPLGGLAQAQTG

SEQ ID NO: 88 - protein, 15 aa, synthetic construct
AQPKGPWAEAQAQTG

SEQ ID NO: 89 - protein, 15 aa, synthetic construct
AQAGGEYEEAQAQTG

SEQ ID NO: 90 - protein, 15 aa, synthetic construct
AQGKGLYEEALAQTG

SEQ ID NO: 91 - protein, 15 aa, synthetic construct
AQAKGDYEAALAQTG

SEQ ID NO: 92 - protein, 15 aa, synthetic construct
AKPPSPWAKAQAQTG

SEQ ID NO: 93 - protein, 15 aa, synthetic construct
AQGGGDYEAALAQTG

SEQ ID NO: 94 - protein, 15 aa, synthetic construct
AQGKGDYEAALAQTG

SEQ ID NO: 95 - protein, 15 aa, synthetic construct
AKPPPLLLLLQAQTG

SEQ ID NO: 96 - protein, 15 aa, synthetic construct
AKPPGPFAKAQAQTG

SEQ ID NO: 97 - protein, 15 aa, synthetic construct
AQPKGPYAEAQAQTG

SEQ ID NO: 98 - protein, 15 aa, synthetic construct
AQPPGPYAEAQAQTG

SEQ ID NO: 99 - protein, 15 aa, synthetic construct
AQGGAEVLTAQAQTG

SEQ ID NO: 100 - protein, 15 aa, synthetic construct
AQGKGLYEAVQAQTG

SEQ ID NO: 101 - protein, 15 aa, synthetic construct
AKPPGPWAKAQAQTG

SEQ ID NO: 102 - protein, 15 aa, synthetic construct
AKPPGPFAEAQAQTG

SEQ ID NO: 103 - protein, 15 aa, synthetic construct
AQAGGDYEAALAQTG

SEQ ID NO: 104 - protein, 15 aa, synthetic construct
AKPPSPFAEAQAQTG

SEQ ID NO: 105 - protein, 15 aa, synthetic construct
AQNKKMYYYAQAQTG

SEQ ID NO: 106 - protein, 15 aa, synthetic construct
AQGGGDYEAVQAQTG

SEQ ID NO: 107 - protein, 15 aa, synthetic construct
AQGKGLYEEVQAQTG

SEQ ID NO: 108 - protein, 15 aa, synthetic construct
AKPPSPFAKAQAQTG

SEQ ID NO: 109 - protein, 15 aa, synthetic construct
AQPPLGGGLAQAQTG

SEQ ID NO: 110 - protein, 15 aa, synthetic construct
AQGGGEYEEALAQTG

SEQ ID NO: 111 - protein, 15 aa, synthetic construct
AQAKGLYEAALAQTG

SEQ ID NO: 112 - protein, 15 aa, synthetic construct
AQGGGEYEEAQAQTG

SEQ ID NO: 113 - protein, 15 aa, synthetic construct
AQGKGGYWYGQAQTG

SEQ ID NO: 114 - protein, 15 aa, synthetic construct
AQAKGLYEEAQAQTG

SEQ ID NO: 115 - protein, 15 aa, synthetic construct
AQAKGLYEEVQAQTG

SEQ ID NO: 116 - protein, 15 aa, synthetic construct
AQGKGLYEEAQAQTG

SEQ ID NO: 117 - protein, 15 aa, synthetic construct
AQGKGDYEAVQAQTG

SEQ ID NO: 118 - protein, 15 aa, synthetic construct
AQAKGLYEEALAQTG

SEQ ID NO: 119 - protein, 15 aa, synthetic construct
AKPPGPYAEAQAQTG

SEQ ID NO: 120 - protein, 15 aa, synthetic construct
AQAKGLYEAAQAQTG

SEQ ID NO: 121 - protein, 15 aa, synthetic construct
AQNMTAAHYAQAQTG

SEQ ID NO: 122 - protein, 15 aa, synthetic construct
AQAGGEYEEALAQTG

SEQ ID NO: 123 - protein, 15 aa, synthetic construct
AQNDKMYWYGQAQTG

SEQ ID NO: 124 - protein, 15 aa, synthetic construct
AQNDKMYEAAQAQTG

SEQ ID NO: 125 - protein, 15 aa, synthetic construct
AQGKGLYEAAQAQTG

SEQ ID NO: 126 - protein, 15 aa, synthetic construct
AQAKGDYEAAQAQTG

SEQ ID NO: 127 - protein, 15 aa, synthetic construct
AQGGGDYDAALAQTG

SEQ ID NO: 128 - protein, 15 aa, synthetic construct
AQAGGDYDAALAQTG

SEQ ID NO: 129 - protein, 15 aa, synthetic construct
AQAKGDYEAVQAQTG

SEQ ID NO: 130 - protein, 15 aa, synthetic construct
AQAKNPAQPALAQTG

SEQ ID NO: 131 - protein, 15 aa, synthetic construct
AQEDKVYDAAQAQTG

SEQ ID NO: 132 - protein, 15 aa, synthetic construct
AQGKGLYEAALAQTG

SEQ ID NO: 133 - protein, 15 aa, synthetic construct
AQGKGDYEAAQAQTG

SEQ ID NO: 134 - protein, 15 aa, synthetic construct
AQNGGMYAAAQAQTG

SEQ ID NO: 135 - protein, 15 aa, synthetic construct
AQNGKGYAYAQAQTG

SEQ ID NO: 136 - protein, 15 aa, synthetic construct
AQAGGEYEEVQAQTG

SEQ ID NO: 137 - protein, 15 aa, synthetic construct
AQGGKGYAYAQAQTG

SEQ ID NO: 138 - protein, 15 aa, synthetic construct
AKPPGPYAKAQAQTG

SEQ ID NO: 139 - protein, 15 aa, synthetic construct
AKPPSPYAEAQAQTG

SEQ ID NO: 140 - protein, 15 aa, synthetic construct
AQPPGPAAEAQAQTG

SEQ ID NO: 141 - protein, 15 aa, synthetic construct
AQPPGPYAAAQAQTG

SEQ ID NO: 142 - protein, 15 aa, synthetic construct
AQPPGPAAVAQAQTG

SEQ ID NO: 143 - protein, 15 aa, synthetic construct
AKPPSPYAKAQAQTG

SEQ ID NO: 144 - protein, 15 aa, synthetic construct
AQGKNPAQPAQAQTG

SEQ ID NO: 145 - protein, 15 aa, synthetic construct
AQAGGDYEAAQAQTG

SEQ ID NO: 146 - protein, 15 aa, synthetic construct
AQNGKMYYYAQAQTG

SEQ ID NO: 147 - protein, 15 aa, synthetic construct
AQGGGDYDAVQAQTG

SEQ ID NO: 148 - protein, 15 aa, synthetic construct
AQNKKMYAAAQAQTG

SEQ ID NO: 149 - protein, 15 aa, synthetic construct
AQGKGGYEAAQAQTG

SEQ ID NO: 150 - protein, 15 aa, synthetic construct
AKPPSPSAKAQAQTG

SEQ ID NO: 151 - protein, 15 aa, synthetic construct
AQGGKGYEYAQAQTG

SEQ ID NO: 152 - protein, 15 aa, synthetic construct
AKPPGPAAKAQAQTG

SEQ ID NO: 153 - protein, 15 aa, synthetic construct
AKPPGPSAEAQAQTG

SEQ ID NO: 154 - protein, 15 aa, synthetic construct
AKPPSPAAEAQAQTG

SEQ ID NO: 155 - protein, 15 aa, synthetic construct
AQAGGDYDAAQAQTG

SEQ ID NO: 156 - protein, 15 aa, synthetic construct
AKPPGPSAKAQAQTG

SEQ ID NO: 157 - protein, 15 aa, synthetic construct
AQGKNPAQPALAQTG

SEQ ID NO: 158 - protein, 15 aa, synthetic construct
AQGNNPAKPALAQTG

SEQ ID NO: 159 - protein, 15 aa, synthetic construct
AQGGGDYEAAQAQTG

SEQ ID NO: 160 - protein, 15 aa, synthetic construct
AQGKNPAQPVQAQTG

SEQ ID NO: 161 - protein, 15 aa, synthetic construct
AKPPGPAAEAQAQTG

SEQ ID NO: 162 - protein, 15 aa, synthetic construct
ATAPSGGGEGQAQTG

SEQ ID NO: 163 - protein, 15 aa, synthetic construct
AKPPSPAAKAQAQTG

SEQ ID NO: 164 - protein, 15 aa, synthetic construct
AQPKGPAAEAQAQTG

SEQ ID NO: 165 - protein, 15 aa, synthetic construct
AQPPGPAAAAQAQTG

SEQ ID NO: 166 - protein, 15 aa, synthetic construct
AQGNNPAKPVQAQTG

SEQ ID NO: 167 - protein, 15 aa, synthetic construct
AKPPSPSAEAQAQTG

SEQ ID NO: 168 - protein, 15 aa, synthetic construct
AQGGGDYDAAQAQTG

SEQ ID NO: 169 - protein, 15 aa, synthetic construct
AQANNPAKPVQAQTG

SEQ ID NO: 170 - protein, 15 aa, synthetic construct
AHADERADTAQAQTG

SEQ ID NO: 171 - protein, 15 aa, synthetic construct
AQANNPAKPALAQTG

SEQ ID NO: 172 - protein, 15 aa, synthetic construct
ASAPPKEDSSQAQTG

SEQ ID NO: 173 - protein, 15 aa, synthetic construct
AQAKNPAQPAQAQTG

SEQ ID NO: 174 - protein, 15 aa, synthetic construct
AQAKNPAQPVQAQTG

SEQ ID NO: 175 - protein, 15 aa, synthetic construct
AQGNNPAKPAQAQTG

SEQ ID NO: 176 - protein, 15 aa, synthetic construct
AQANNPAKPAQAQTG

SEQ ID NO: 177 - protein, 15 aa, synthetic construct
AQEGAESDSAQAQTG

SEQ ID NO: 178 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 305 aa, synthetic construct
MQLLLALLALAYVAPSTEDSGWCYEIQTKDPRSSCLGPEKWPGACKENQQSPINIVTARTKVNPRLTPFILVGYDQKQQWPIKNNQHTVEMTLGGGACIIGGDLPARYEAVQLHLHWSNGNDNGSEHSIDGRHFAMEMHIVHKKLTSSKEDSKDKFAVLAFMIEVGDKVNKGFQPLVEALPSISKPHSTSTVRESSLQDMLPPSTKMYTYFRYNGSLTTPNCDETVIWTVYKQPIKIHKNQFLEFSKNLYYDEDQKLNMKDNVRPLQPLGKRQVFKSHAPGQLLSLPLPTLLVPTLTCLVANFLQ

SEQ ID NO: 180 - protein, 312 aa, synthetic construct
MRMLLALLALSAARPSASAESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKSGAPGRPLPWALPALLGPMLACLLAGFLR

SEQ ID NO: 181 - protein, 4 aa, synthetic construct
KPTP

SEQ ID NO: 182 - protein, 4 aa, synthetic construct
PTPL

SEQ ID NO: 183 - protein, 4 aa, synthetic construct
TPLL

SEQ ID NO: 184 - protein, 4 aa, synthetic construct
PLLG

SEQ ID NO: 185 - protein, 4 aa, synthetic construct
LLGL

SEQ ID NO: 186 - protein, 4 aa, synthetic construct
LGLL

SEQ ID NO: 187 - protein, 4 aa, synthetic construct
GLLQ

SEQ ID NO: 188 - protein, 5 aa, synthetic construct
KPTPL

SEQ ID NO: 189 - protein, 5 aa, synthetic construct
PTPLL

SEQ ID NO: 190 - protein, 5 aa, synthetic construct
TPLLG

SEQ ID NO: 191 - protein, 5 aa, synthetic construct
PLLGL

SEQ ID NO: 192 - protein, 5 aa, synthetic construct
LLGLL

SEQ ID NO: 193 - protein, 5 aa, synthetic construct
LGLLQ

SEQ ID NO: 194 - protein, 6 aa, synthetic construct
KPTPLL

SEQ ID NO: 195 - protein, 6 aa, synthetic construct
PTPLLG

SEQ ID NO: 196 - protein, 6 aa, synthetic construct
TPLLGL

SEQ ID NO: 197 - protein, 6 aa, synthetic construct
PLLGLL

SEQ ID NO: 198 - protein, 6 aa, synthetic construct
LLGLLQ

SEQ ID NO: 199 - protein, 7 aa, synthetic construct
KPTPLLG

SEQ ID NO: 200 - protein, 7 aa, synthetic construct
PTPLLGL

SEQ ID NO: 201 - protein, 7 aa, synthetic construct
TPLLGLL

SEQ ID NO: 202 - protein, 7 aa, synthetic construct
PLLGLLQ

SEQ ID NO: 203 - protein, 8 aa, synthetic construct
KPTPLLGL

SEQ ID NO: 204 - protein, 8 aa, synthetic construct
PTPLLGLL

SEQ ID NO: 205 - protein, 8 aa, synthetic construct
TPLLGLLQ

SEQ ID NO: 206 - protein, 9 aa, synthetic construct
KPTPLLGLL

SEQ ID NO: 207 - protein, 9 aa, synthetic construct
PTPLLGLLQ

SEQ ID NO: 208 - protein, 4 aa, synthetic construct
PLLL

SEQ ID NO: 209 - protein, 4 aa, synthetic construct
LLLL

SEQ ID NO: 210 - protein, 4 aa, synthetic construct
LLLQ

SEQ ID NO: 211 - protein, 5 aa, synthetic construct
TPLLL

SEQ ID NO: 212 - protein, 5 aa, synthetic construct
PLLLL

SEQ ID NO: 213 - protein, 5 aa, synthetic construct
LLLLL

SEQ ID NO: 214 - protein, 5 aa, synthetic construct
LLLLQ

SEQ ID NO: 215 - protein, 6 aa, synthetic construct
PTPLLL

SEQ ID NO: 216 - protein, 6 aa, synthetic construct
TPLLLL

SEQ ID NO: 217 - protein, 6 aa, synthetic construct
PLLLLL

SEQ ID NO: 218 - protein, 6 aa, synthetic construct
LLLLLQ

SEQ ID NO: 219 - protein, 7 aa, synthetic construct
KPTPLLL

SEQ ID NO: 220 - protein, 7 aa, synthetic construct
PTPLLLL

SEQ ID NO: 221 - protein, 7 aa, synthetic construct
TPLLLLL

SEQ ID NO: 222 - protein, 7 aa, synthetic construct
PLLLLLQ

SEQ ID NO: 223 - protein, 8 aa, synthetic construct
KPTPLLLL

SEQ ID NO: 224 - protein, 8 aa, synthetic construct
PTPLLLLL

SEQ ID NO: 225 - protein, 8 aa, synthetic construct
TPLLLLLQ

SEQ ID NO: 226 - protein, 9 aa, synthetic construct
KPTPLLLLL

SEQ ID NO: 227 - protein, 9 aa, synthetic construct
PTPLLLLLQ

SEQ ID NO: 228 - protein, 4 aa, synthetic construct
PPLG

SEQ ID NO: 229 - protein, 4 aa, synthetic construct
PLGG

SEQ ID NO: 230 - protein, 4 aa, synthetic construct
LGGL

SEQ ID NO: 231 - protein, 4 aa, synthetic construct
GGLL

SEQ ID NO: 232 - protein, 4 aa, synthetic construct
GLLA

SEQ ID NO: 233 - protein, 4 aa, synthetic construct
LLAQ

SEQ ID NO: 234 - protein, 5 aa, synthetic construct
PPLGG

SEQ ID NO: 235 - protein, 5 aa, synthetic construct
PLGGL

SEQ ID NO: 236 - protein, 5 aa, synthetic construct
LGGLL

SEQ ID NO: 237 - protein, 5 aa, synthetic construct
GGLLA

SEQ ID NO: 238 - protein, 5 aa, synthetic construct
GLLAQ

SEQ ID NO: 239 - protein, 6 aa, synthetic construct
PPLGGL

SEQ ID NO: 240 - protein, 6 aa, synthetic construct
PLGGLL

SEQ ID NO: 241 - protein, 6 aa, synthetic construct
LGGLLA

SEQ ID NO: 242 - protein, 6 aa, synthetic construct
GGLLAQ

SEQ ID NO: 243 - protein, 7 aa, synthetic construct
PPLGGLL

SEQ ID NO: 244 - protein, 7 aa, synthetic construct
PLGGLLA

SEQ ID NO: 245 - protein, 7 aa, synthetic construct
LGGLLAQ

SEQ ID NO: 246 - protein, 8 aa, synthetic construct
PPLGGLLA

SEQ ID NO: 247 - protein, 8 aa, synthetic construct
PLGGLLAQ

SEQ ID NO: 248 - protein, 4 aa, synthetic construct
KPPG

SEQ ID NO: 249 - protein, 4 aa, synthetic construct
PPGP

SEQ ID NO: 250 - protein, 4 aa, synthetic construct
PGPW

SEQ ID NO: 251 - protein, 4 aa, synthetic construct
GPWA

SEQ ID NO: 252 - protein, 4 aa, synthetic construct
PWAE

SEQ ID NO: 253 - protein, 4 aa, synthetic construct
WAEA

SEQ ID NO: 254 - protein, 4 aa, synthetic construct
AEAQ

SEQ ID NO: 255 - protein, 5 aa, synthetic construct
KPPGP

SEQ ID NO: 256 - protein, 5 aa, synthetic construct
PPGPW

SEQ ID NO: 257 - protein, 5 aa, synthetic construct
PGPWA

SEQ ID NO: 258 - protein, 5 aa, synthetic construct
GPWAE

SEQ ID NO: 259 - protein, 5 aa, synthetic construct
PWAEA

SEQ ID NO: 260 - protein, 5 aa, synthetic construct
WAEAQ

SEQ ID NO: 261 - protein, 6 aa, synthetic construct
KPPGPW

SEQ ID NO: 262 - protein, 6 aa, synthetic construct
PPGPWA

SEQ ID NO: 263 - protein, 6 aa, synthetic construct
PGPWAE

SEQ ID NO: 264 - protein, 6 aa, synthetic construct
GPWAEA

SEQ ID NO: 265 - protein, 6 aa, synthetic construct
PWAEAQ

SEQ ID NO: 266 - protein, 7 aa, synthetic construct
KPPGPWA

SEQ ID NO: 267 - protein, 7 aa, synthetic construct
PPGPWAE

SEQ ID NO: 268 - protein, 7 aa, synthetic construct
PGPWAEA

SEQ ID NO: 269 - protein, 7 aa, synthetic construct
GPWAEAQ

SEQ ID NO: 270 - protein, 8 aa, synthetic construct
KPPGPWAE

SEQ ID NO: 271 - protein, 8 aa, synthetic construct
PPGPWAEA

SEQ ID NO: 272 - protein, 8 aa, synthetic construct
PGPWAEAQ

SEQ ID NO: 273 - protein, 9 aa, synthetic construct
KPPGPWAEA

SEQ ID NO: 274 - protein, 9 aa, synthetic construct
PPGPWAEAQ

SEQ ID NO: 275 - protein, 4 aa, synthetic construct
PPLL

SEQ ID NO: 276 - protein, 4 aa, synthetic construct
LLGG

SEQ ID NO: 277 - protein, 4 aa, synthetic construct
GGLA

SEQ ID NO: 278 - protein, 4 aa, synthetic construct
GLAQ

SEQ ID NO: 279 - protein, 5 aa, synthetic construct
PPLLG

SEQ ID NO: 280 - protein, 5 aa, synthetic construct
PLLGG

SEQ ID NO: 281 - protein, 5 aa, synthetic construct
LLGGL

SEQ ID NO: 282 - protein, 5 aa, synthetic construct
LGGLA

SEQ ID NO: 283 - protein, 5 aa, synthetic construct
GGLAQ

SEQ ID NO: 284 - protein, 6 aa, synthetic construct
PPLLGG

SEQ ID NO: 285 - protein, 6 aa, synthetic construct
PLLGGL

SEQ ID NO: 286 - protein, 6 aa, synthetic construct
LLGGLA

SEQ ID NO: 287 - protein, 6 aa, synthetic construct
LGGLAQ

SEQ ID NO: 288 - protein, 7 aa, synthetic construct
PPLLGGL

SEQ ID NO: 289 - protein, 7 aa, synthetic construct
PLLGGLA

SEQ ID NO: 290 - protein, 7 aa, synthetic construct
LLGGLAQ

SEQ ID NO: 291 - protein, 8 aa, synthetic construct
PPLLGGLA

SEQ ID NO: 292 - protein, 8 aa, synthetic construct
PLLGGLAQ

SEQ ID NO: 293 - protein, 131 aa, synthetic construct
MDTSHTTKSCVLILLVALLCAERAQGLQCYECYGVPIETSCPAVTCRASDGFCIAQNIELIEDSQRRKLKTRQCLSFCPAGVPIRDPNIRERTSCCSEDLCNAAVPTAGSTWTMAGVLLFSLSSVVLQTLL

(19) United States
(12) Patent Application Publication
(10) Pub. No.: US 2023/0357434 A1
(43) Pub. Date: Nov. 9, 2023

(54) COMPOSITIONS AND METHODS FOR CROSSING BLOOD BRAIN BARRIER

(71) Applicant: California Institute of Technology, Pasadena, CA (US)

(72) Inventors: Timothy F. Shay, Studio City, CA (US); Viviana Gradinaru, La Canada Flintridge, CA (US); Xiaozhe Ding, Pasadena, CA (US)

(21) Appl. No.: 18/177,566

(22) Filed: Mar. 2, 2023

Related U.S. Application Data
(60) Provisional application No. 63/316,298, filed on Mar. 3, 2022.
(60) Provisional application No. 63/359,168, filed on Jul. 7, 2022.
(60) Provisional application No. 63/480,279, filed on Jan. 17, 2023.

(51) Int. Cl. C07K 16/40; C12N 15/86; A61P 25/00

(52) U.S. Cl. CPC C07K 16/40; C12N 15/86; A61P 25/00; C12N 2750/14143; C07K 2317/31

(57) ABSTRACT

Disclosed herein include novel blood-brain barrier (BBB)-crossing receptors on the BBB interface, targeting peptides and derivatives thereof capable of binding to the novel receptors, and related methods of using the receptors to increase the permeability of the BBB and to deliver an agent to a nervous system (e.g., CNS). In some embodiments, the BBB-crossing receptor is carbonic anhydrase IV. Disclosed herein also include recombinant adeno-associated viruses (rAAVs) with increased specificity and transduction efficiency across the BBB and related compositions and methods of treating various diseases and conditions.

### RELATED APPLICATIONS

This application claims the benefit under 35 U.S.C. § 119(e) of U.S. Provisional Patent Application No. 63/316,298, filed on Mar. 3, 2022, U.S. Provisional Patent Application No. 63/359,168, filed on Jul. 7, 2022, and U.S. Provisional Patent Application No. 63/480,279, filed on Jan. 17, 2023. The content of these related applications is incorporated herein by reference in its entirety for all purposes.

### STATEMENT REGARDING FEDERALLY SPONSORED R&D

This invention was made with government support under Grant No. NS111369 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-302459-US-SeqList, created Mar. 2, 2023, which is 258 kilobytes in size. The information in the electronic format of the Sequence Listing is incorporated herein by reference in its entirety.

### BACKGROUND

### Field

The present disclosure relates to methods and targeting peptides for crossing the blood brain barrier.

### Description of the Related Art

The blood brain barrier (BBB) presents a fundamental bottleneck to the development of effective research tools and therapeutics for the central nervous system (CNS). This structure, comprising mainly of brain endothelial cells, requires large molecules to be delivered via invasive intracranial injections, technically challenging focused ultrasound, or receptor-mediated transcytosis. The rational design of BBB-crossing large molecules has long been hampered by the imperfect understanding of the mechanisms involved in transcytosis, with only a handful of targets, such as the transferrin receptor, validated for research and therapies.

Thus, the identification of BBB-crossing targets, mechanisms, molecules and methods is needed to improve the efficiencies of research tools and therapies for CNS.

### SUMMARY

Disclosed herein includes a method of increasing permeability of the blood brain barrier. The method, in some embodiments, comprises: providing a targeting peptide capable of binding to a carbonic anhydrase IV, thereby increasing permeability of the blood brain barrier. In some embodiments, at least one activity of the carbonic anhydrase IV is reduced through binding to a targeting peptide. Also provided rein includes a method of increasing permeability of the blood brain barrier. The method, in some embodiments, comprises: reducing carbonic anhydrase IV activity, thereby increasing permeability of the blood brain barrier. In the methods disclosed herein, the targeting peptide can, for example, bind a zinc binding site and/or hydrophobic substrate binding pocket of the carbonic anhydrase IV. In some embodiments the permeability of the blood brain barrier is increase by at least 25%, 50%, 75%, 100%, or more as compared to the absence of the target peptide or the reduction of carbonic anhydrase IV activity. Also provides herein includes a method of delivering a payload to a nervous system. The method, in some embodiments, comprises: providing a targeting peptide capable of binding to a carbonic anhydrase IV or a derivative thereof, wherein the targeting peptide is part of a delivery system, and wherein the delivery system comprises the payload to be delivered to a nervous system; and administering the delivery system to the subject.

As disclosed herein, the delivery system can, for example, comprise nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a viral vector or a non-viral vector. The viral vector can, for example, comprise an adenovirus vector, an AAV vector, a lentiviral vector, or a retrovirus vector. In some embodiments, the target peptide is part of a capsid protein of an AAV vector. The AAV vector can be a vector selected from AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, and a variant thereof. In some embodiments, the AAV vector is AAV 9. The non-viral vector can, for example, comprise lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

In some embodiments, the payload to be delivered to a nervous system is a biological molecule, a non-biological molecule, or a combination thereof. The biological molecule can be a nucleic acid sequence, a protein, a peptide, a lipid, a polysaccharide, or a combination thereof. In some embodiments, the payload is a therapeutic molecule. In some embodiments, the nucleic acid sequence to be delivered to a nervous system comprises one or more of: a) a trophic factor, a growth factor, or other soluble factors that might be released from the transduced cells and affect the survival or function of that cell and/or surrounding cells; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid used for assessing the state of a cell; e) a cDNA and/or associated guide RNA for performing genomic engineering; f) a sequence for genome editing via homologous recombination; g) a DNA sequence encoding a therapeutic RNA; h) an shRNA or an artificial miRNA delivery system; or i) a DNA sequence that influences the splicing of an endogenous gene.

In some embodiments, the carbonic anhydrase IV is a mouse carbonic anhydrase IV. In some embodiments, the carbonic anhydrase IV has an amino acid sequence having at least 80% sequence identity to an amino acid sequence of SEQ ID NO: 179. In some embodiments, the carbonic anhydrase IV is a human carbonic anhydrase IV. In some embodiments, the carbonic anhydrase IV has an amino acid sequence having at least 80% sequence identity to an amino acid sequence of SEQ ID NO: 180. In some embodiments, upon binding the targeting peptide is capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179; or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180. In some embodiments, the targeting peptide comprises (1) an amino acid sequence selected from the group consisting of SEQ ID NOs: 70-174; or (2) an amino acid sequence having at least 80% sequence identity to an amino acid sequence selected from the group consisting of SEQ ID NOs: 70-174. In some embodiments, the targeting peptide comprises an amino acid sequence selected from SEQ ID NOs: 70-108. In some embodiments, the targeting peptide comprises an amino acid sequence selected from SEQ ID NOs: 70-86. In some embodiments, the targeting peptide comprises (1) an amino acid sequence selected from the group consisting of: AKPTPLLGLLQAQTG (SEQ ID NO: 70), AKPTPLLLLLQAQTG (SEQ ID NO: 71), AQPPLGGLLAQAQTG (SEQ ID NO: 72), AKPPGPWAEAQAQTG (SEQ ID NO: 73), and AQPPLLGGLAQAQTG (SEQ ID NO: 74); or (2) an amino acid sequence having at least 80% sequence identity to an amino acid sequence selected from the group consisting of SEQ ID NOs: 70-74. In some embodiments, the targeting peptide is inserted between two adjacent amino acids in AA587-594 of SEQ ID NO: 178 of the AAV9 vector or functional equivalents of AA587-594 in an amino acid sequence at least 80% identical to SEQ ID NO: 178. In some embodiments, the targeting peptide is inserted between AA588-589 of SEQ ID NO: 178 of the AAV9 vector or functional equivalents of AA588-589 in an amino acid sequence at least 80% identical to SEQ ID NO: 178. The AAV vector can be, for example, conjugated to a nanoparticle, a second molecule, or a combination thereof.

The administration can be a systemic administration. In some embodiments, the administration is an intravenous administration. In some embodiments, the administration is an intrathecal administration. The subject can be, for example, a mammal. In some embodiments, the subject is a human. In some embodiments, the subject is a subject suffering from or at a risk to develop one or more of chronic pain, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. The lysosomal storage disorder that involve cells within the CNS can be, for example, Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. In some embodiments, the subject is a subject suffering from, at risk to develop, or has suffered from a stroke, traumatic brain injury, epilepsy, or spinal cord injury.

Disclosed herein includes an AAV capsid protein comprising a targeting peptide having a binding specificity to a carbonic anhydrase IV. In some embodiments, the targeting peptide is part of a capsid protein of the rAAV vector. In some embodiments, the targeting peptide is inserted between two adjacent amino acids in AA587-594 of SEQ ID NO: 178 or functional equivalents of AA587-594 in an amino acid sequence at least 80% identical to SEQ ID NO: 178. In some embodiments, the targeting peptide comprises (1) an amino acid sequence selected from the group consisting of SEQ ID NOs: 70-174; (2) an amino acid sequence having at least 80% sequence identity to an amino acid sequence selected from the group consisting of SEQ ID NOs: 70-174. The AAV can be, for example, AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, or a variant thereof. In some embodiments, the AAV vector is AAV9.

Disclosed herein includes a recombinant adeno-associated virus (rAAV), comprising any of the AAV capsid proteins disclosed herein. Disclosed herein includes a rAAV, comprising an AAV capsid protein which comprises a targeting peptide having a binding specificity to a carbonic anhydrase IV, wherein the amino acid sequence of the targeting peptide is inserted between two adjacent amino acids in AA587-594, or functional equivalents thereof, of the AAV capsid protein. In some embodiments, the two adjacent amino acids are AA588 and AA589. In some embodiments, the targeting peptide comprises (1) an amino acid sequence selected from SEQ ID NOs: 70-174; or (2) an amino acid sequence having at least 80% sequence identity to an amino acid sequence selected from SEQ ID NOs: 70-174.

Disclosed herein includes a composition for use in the delivery of an agent to a nervous system of a subject in need. The composition, in some embodiments, comprises an AAV comprising (1) any of the AAV capsid proteins disclosed herein and (2) an agent to be delivered to the nervous system of the subject. In some embodiments, the nervous system is the central nervous system (CNS), the peripheral nervous system (PNS), or a combination thereof. The nervous system can be, for example, brain endothelial cells, neurons, capillaries in the brain, arterioles in the brain, arteries in the brain, or a combination thereof. In some embodiments, the composition is a pharmaceutical composition comprising one or more pharmaceutical acceptable carriers. In some embodiments, the agent to be delivered comprises a nucleic acid, a peptide, a small molecule, an aptamer, or a combination thereof.

Disclosed herein includes an antibody or fragment thereof, wherein the antibody or fragment thereof comprises an amino acid sequence having a binding specificity to a carbonic anhydrase IV. In some embodiments, the antibody or fragment thereof comprises (1) an amino acid sequence selected from the group consisting of SEQ ID NOs: 70-174; or (2) an amino acid sequence having at least 80% sequence identity to an amino acid sequence selected from the group consisting of SEQ ID NOs: 70-174. In some embodiments, the antibody or fragment thereof is a bispecific antibody comprising at least one Fab having specificity to the carbonic anhydrase IV. Disclosed herein includes an antibody conjugate comprising any one of the antibodies disclosed herein or fragment thereof. In some embodiments, the antibody conjugate further comprises a therapeutic agent or a detectable label.

Disclosed herein includes a peptide or a derivative or a conjugate thereof, having specificity to a carbonic anhydrase IV. Disclosed herein includes a nucleic acid comprising a sequence encoding any one of the antibodies disclosed herein or fragment thereof, or any of the peptides disclosed herein or the derivative or the conjugate thereof.

Disclosed herein includes a delivery system, comprising (1) a targeting peptide having specificity to a carbonic anhydrase IV; and (2) an agent. In some embodiments, the targeting peptide is (1) displayed on the surface of the delivery system; or (2) partially embedded in the delivery system. In some embodiments, the delivery system is selected from nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a viral vector or a non-viral vector. In some embodiments, the delivery system comprises a nanoparticle selected from: lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

Disclosed herein includes a method of designing a targeting peptide having specificity to a carbonic anhydrase IV. The method, in some embodiments, comprises: generating in silico one or more targeting peptides each capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179, or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180. In some embodiments, generating in silico the one or more targeting peptides comprises: generating in silico a plurality of candidate peptides; performing computer-assisted docking simulations for each of the plurality of candidate peptides binding to the carbonic anhydrase IV; and analyzing the structure of the carbonic anhydrase IV binding to one or more of the plurality of candidate peptides to identify one or more targeting peptides capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179, or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180. In some embodiments, the method comprises: obtaining a binding score for each of the plurality of candidate peptides binding to the carbonic anhydrase IV; and selecting one or more of the plurality of candidate peptides having a binding score above a threshold value as a targeting peptide having specificity to the carbonic anhydrase IV. In some embodiments, the method comprises: comparing the binding scores of two or more of the plurality of candidate peptides to rank the candidate peptide sequences. In some embodiments, obtaining the binding score for each of the plurality of candidate peptide sequences comprises (1) counting a total number of atoms in the interface of a candidate peptide and the carbonic anhydrase IV; (2) counting a total number of atoms in the candidate peptide, wherein the atoms are clashing with the carbonic anhydrase IV; (3) obtaining a binding angle of the candidate peptide; and (4) obtaining a binding depth of the candidate peptide.

Disclosed herein includes a method of increasing permeability of the blood brain barrier in a murine species. The method, in some embodiments, comprises: providing a targeting peptide capable of binding to Ly6c1, thereby increasing permeability of the blood brain barrier in the murine species. In some embodiments, the targeting peptide has 1) an amino acid sequence selected from the group consisting of AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), and SPRYKGDSVAQ (SEQ ID NO: 57); or (2) an amino acid sequence having at least 80% sequence identity to an amino acid sequence selected from the group consisting of AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), and SPRYKGDSVAQ (SEQ ID NO: 57). In some embodiments, the targeting peptide has an amino acid sequence of SPRYKGDSVAQ (SEQ ID NO: 57) or an amino acid sequence having at least 80% identity to SEQ ID NO: 57. In some embodiments, the targeting peptide is inserted between two adjacent amino acids in AA587-590 of SEQ ID NO: 178 or functional equivalents of AA587-590 in an amino acid sequence at least 80% identical to SEQ ID NO: 178.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1A-FIG. 1C depict non-limiting exemplary embodiments and data related to the identification of engineered AAVs that did not utilize Ly6a for BBB crossing. FIG. 1A depicts that AAV-PHP.eB can efficiently cross the BBB using membrane-localized Ly6a in C57BL/6J mice but not GPI-disrupted Ly6a in BALB/cJ mice. FIG. 1B depicts a clustering analysis of CNS-tropic engineered AAV insertion sequences. The thickness of connecting lines represents the degree of relatedness between nodes. AAVs in the box in the top left corner showed a high degree of relatedness to PHP.eB. AAVs outside the box showed little relation to PHP.eB or each other. The AA sequences inserted between positions 588-589 and 452-458 of the AAV9 capsid for each variant are shown. FIG. 1C depicts surface plasmon resonance (SPR) of engineered AAVs binding to Ly6a-Fc captured on a protein A chip. Data are representative of 2 independent experiments.

FIG. 2A-FIG. 2C depict non-limiting exemplary embodiments and data related to the performance of Ly6a-independent AAVs across mouse strains. FIG. 2A depicts transduction by AAV-PHP.eB, AAV-PHP.N, AAV-PHP.C1-C4, and AAV-F in the thalamus brain region of 129S1/SvlmJ (membrane-localized Ly6a), CBA/J (GPI-disrupted Ly6a), DBA/2J (membrane-localized Ly6a), and NOD/ShiLtJ (GPI-disrupted Ly6a) mice shown from sagittal brain sections. All images were acquired under the same image settings, except the bottom left quadrant of PHP.eB images across strains, which were optimized for DBA/2J. The indicated capsids were used to package ssAAV:CAG-mNeonGreen and administered to 6-8-week-old adult mice (n=2 per group, 1×10¹¹ viral genomes (v.g.) delivered i.v. per mouse, 3 weeks of expression). All images were from one experiment, in which all viruses were freshly prepared and titered in the same assay to ensure consistent dosing. FIG. 2B depicts a mouse phylogenetic tree including the strains used. FIG. 2C depicts thalamus mean pixel intensity across animals. Each data point represents one animal and bars indicate the mean values.

FIG. 3A-FIG. 3G depict non-limiting exemplary embodiments and data related to novel receptors enhancing the infectivity of engineered AAVs in vitro. FIG. 3A depicts a plot of membrane protein differential expression score in endothelial cells calculated in Scanpy versus mean transcript abundance in endothelial cells constructed from single-cell RNA sequencing of C57BL/6J cortex. Proteins selected for cell culture screening are indicated by larger dots (Table 3) and hits are labeled and marked with crosses. FIG. 3B depicts the screening of potential receptors in cell culture by comparing AAV fluorescent protein transgene levels at low multiplicity of infection (MOI) in mock-transfected cells and cells transfected with each potential receptor. FIG. 3C depicts dose dependence of AAV9 and PHP.eB packaging CAG-mNeonGreen in HEK293T cells in 96-well plates. At 1×10⁹ v.g. per well, PHP.eB had remarkably higher potency than AAV9 in Ly6a-transfected HEK293T cells. Scales show the extent of infection (Max: 0.75, Min: 0.03) and total brightness per signal area (Max: 0.79, Min: 0.39). FIG. 3D depicts the potency of engineered AAVs for HEK293T cells transfected with the potential receptor panel. Extent of infection (Left, Max: 0.33, Min: 0.001; Right, Max: 0.55, Min: 0.03). Total brightness per signal area (Left, Max: 0.55, Min: 0.06; Right, Max: 0.61, Min: 0.11) FIG. 3E depicts representative images of HEK cells cultured in 96 well plates, either mock-transfected (none) or transfected with a potential receptor, infected with 1×10⁹ v.g. per well of AAVs (the left two columns are Ly6a binding AAVs) packaging CAG-mNeonGreen and imaged 24 hours post-transduction. An overlay of brightfield and fluorescence images is presented. Scale bar=200 μm. FIG. 3F depicts amino acid frequencies by position among engineered AAVs found to have infectivity enhancements with Ly6a (6 viruses), Ly6c1 (SEQ ID NO: 293; 9 viruses), and Car4 (SEQ ID NO: 179; 2 viruses). FIG. 3G depicts the potency of engineered AAVs for HEK293T cells transfected with Ly6 and Car family potential receptors. Extent of infection (Left, Max: 0.51, Min: 0.03; Right, Max: 0.52, Min: 0.05). Total brightness per signal area (Left, Max: 0.75, Min: 0.13; Right, Max: 0.79, Min: 0.34).

FIG. 4A-FIG. 4C depict non-limiting exemplary embodiments and data related to the role of carbonic anhydrase IV in the CNS potency of Car4-dependent AAV. FIG. 4A depicts immunostaining for Car4 in the brains of WT/WT and KO/KO Car4 mice. Magnified regions from WT/WT demonstrate endothelial expression across diverse brain regions. FIG. 4B depicts fluorescence images from transgene expression assay throughout the brain and liver of WT/WT Car4 mice administered with AAV-PHP.eB, 9P31, and 9P36. AAV-PHP.eB, 9P31, and 9P36 packaging mNeonGreen under the control of the ubiquitous CAG promoter were intravenously administered to WT/WT Car4 mice at a dose of 3×10¹¹ v.g. per animal (n=3 per condition). Three weeks after administration, transgene expression was assayed by mNeonGreen fluorescence throughout the brain and liver. FIG. 4C depicts that fluorescence images from transgene expression assay throughout the brin and liver of KO/KO Car4 mice administered with AAV-PHP.eB, 9P31, and 9P36. AAV-PHP.eB, 9P31, and 9P36 packaging mNeonGreen under the control of the ubiquitous CAG promoter were intravenously administered to KO/KO Car4 mice at a dose of 3×10¹¹ v.g. per animal (n=3 per condition). Three weeks after administration, transgene expression was assayed by mNeonGreen fluorescence throughout the brain and liver. Scale bars=2 mm.

FIG. 5A-FIG. 5D depict non-limiting exemplary embodiments and data related to the engineering of an enhanced Ly6c1-dependent AAV. FIG. 5A depicts AAV library design strategy. Scanning 3-mer libraries of PHP.C1-C4 were constructed as shown with Xs indicating the position of NNK codons. AAVs were pooled and two rounds of M-CREATE selections were performed in 6-8 week old Cre-transgenic mice. FIG. 5B depicts the round 2 selection of brain enrichments in diverse Cre-transgenic and wild-type mice administrated with selected control AAVs and 20 capsid variants selected for further study. The yield was determined during small-scale production for final screening across strains. FIG. 5C depicts the potency in cell culture infectivity assay of PHP.eB, PHP.C2, and PHP.eC (variant 19 in FIG. 5B) in HEK293T cells transfected with mock (none) or Ly6c1 receptors, demonstrating that PHP.eC retained Ly6c1 interaction. Extent of infection (Max: 0.93, Min: 0.02). Total brightness per signal area (Max: 0.74, Min: 0.16). FIG. 5D depicts that fluorescence images from transgene expression assay throughout the brain and liver of NOD/ShiLtJ and BALB/cJ mice administered with PHP.C2 and PHP.eC. PHP.C2 and PHP.eC packaging mNeonGreen under the control of the ubiquitous CAG promoter were intravenously administered to NOD/ShiLtJ and BALB/cJ mice at a dose of 3×10¹¹ v.g. per animal (n=3 per condition). Three weeks after administration, transgene expression was assayed by mNeonGreen fluorescence throughout the brain and liver, demonstrating PHP.eC's increased potency. Sagittal image scale bars=2 mm. Brain region scale bars=250 μm.

FIG. 6A-FIG. 6G depict non-limiting exemplary embodiments and data related to in silico predictions of engineered AAV receptors and Ly6a interaction complex pose. FIG. 6A depicts an overview of AlphaFold2-based in silico Automated Pairwise Peptide-Receptor Analysis for Screening Engineered AAVs (APPRAISE-AAV). Surface peptides from AAV variants were put in pairwise binding competition using AlphaFold2. A peptide competition metric was calculated according to each peptide's interface energy, binding angle, and pocket depth before being assembled into broader ranked matrices of interaction likelihood. Competition results reflect the relative peptide binding probability encoded in the AlphaFold2 neural network. FIG. 6B depicts a table of engineered AAV capsids, their confirmed receptors, and the capsid peptide sequences used in APPRAISE-AAV. FIG. 6C depicts matrices ranking AAV peptides by their average competition metric over ten replicate conditions for Ly6a, Ly6c1, and mouse Car4. AAV peptide labels in bold indicate those experimentally identified to interact with the corresponding receptor. Metric values out of range (−100-100) were capped to range limits. FIG. 6D depicts AlphaFold2-predicted Ly6a-PHP.eB peptide complex structure. PHP.eB peptide was colored by pLDDT (predicted Local Distance Difference Test) score, a per-residue estimation of the model confidence. The highest confidence side chains, P5′ and F6′, are shown as spheres. Ly6a A58R mutation, chosen to disrupt the predicted peptide interaction, resulted in reduced potency in the cell culture infectivity assay. Extent of infection (Max: 0.29, Min: 0.03). Total brightness per signal area (Max: 0.61, Min: 0.16). FIG. 6E depicts Ly6a residues with at least 2 atoms within 5 angstroms (Å) of the modeled PHP.eB peptide. FIG. 6F depicts the complete model of the PHP.eB trimer and Ly6a complex. The AlphaFold2 structural prediction from FIG. 6D was combined with a capsid monomer-receptor structural prediction and optimized using Rosetta Remodel within the context of the AAV trimer (FIG. 13A). FIG. 6G depicts a zoom-in view of the PHP.eB-Ly6a binding interface in modeled PHP.eB-Ly6a complex and PHP.eB residues with at least 2 atoms within 5 A of Ly6a.

FIG. 7A-FIG. 7D depict non-limiting exemplary embodiments and data related to the interactions of engineered AAVs with carbonic anhydrase IV. FIG. 7A depicts AlphaFold2-predicted mouse Car4-9P31 peptide complex structure. 9P31 peptide was colored by pLDDT score at each residue with the highest confidence side chains shown as spheres. FIG. 7B depicts a cut-away view of mouse Car4 catalytic pocket with modeled 9P31 peptide binding pose (top left) and crystallographic brinzolamide binding pose (PDB ID: 3ZNC, top right). Cell culture infectivity assay of brinzolamide's effects on engineered AAVs (bottom). Extent of infection (Max: 0.63, Min: 0.04). Total brightness per signal area (Max: 0.75, Min: 0.18). FIG. 7C depicts views of amino acid side chains that differ between mouse (PDB ID: 3ZNC) and human (PDB ID: 1ZNC) carbonic anhydrase IV in relation to brinzolamide and 9P31 peptide binding poses. FIG. 7D depicts the potency in cell culture infectivity assay of 9P31 and 9P36 in HEK293T cells transfected with mouse, rhesus macaque, or human carbonic anhydrase IV receptors, as well as two chimeric receptors of mouse and human carbonic anhydrase IV that exchange the loop sequences depicted. Extent of infection (left, Max: 0.52, Min: 0.05, right, Max: 0.65, Min: 0.03). Total brightness per signal area (left, Max: 0.78, Min: 0.46, right, Max: 0.75, Min: 0.13).

FIG. 8A-FIG. 8C depict non-limiting exemplary embodiments and data related to low-dose AAV performance across mouse strains. FIG. 8A depicts low-dose AAV performance across mouse strains that are corresponding to sagittal sections from FIG. 2A of 129S1/SvlmJ, CBA/J, DBA/2J, and NOD/ShiLtJ mice retro-orbitally injected with 1×10¹¹ v.g. per animal of engineered AAV packaging CAG-mNeonGreen (n=2). Mice were 6-8 weeks old at injection and tissue was collected 3 weeks after injection. Scale bars=2 mm. FIG. 8B depicts the quantification of mean fluorescence intensity in the thalamus brain region of male and female 129S1/SvlmJ and CBA/J mice retro-orbitally injected with 1×10¹¹ v.g. per animal of PHP.C1 or PHP.C2 packaging CAG-mNeonGreen (n=2). Mice were 6-8 weeks old at injection. Tissues were collected 3 weeks after injection. Each data point represents one animal and bars indicate the mean values. FIG. 8C depicts AAV-PHP.C3 potency in C57BL/6J, CBA/J, and F1-cross mice. Animals were retro-orbitally injected at 6-8 weeks old with 3×10¹¹ v.g. AAV-PHP.C3 packaging CAG-mNeonGreen. Three weeks after injection, tissues were collected. The thalamus was imaged to assay virus potency.

FIG. 9A-FIG. 9C depict non-limiting exemplary embodiments and data related to representative images from cell culture infectivity assay. FIG. 9A depicts fluorescence images showing the dose dependence of AAV9 and PHP.eB packaging CAG-mNeonGreen in HEK293T cells in 96-well plates. At 1×10⁹ v.g. per well, PHP.eB has a remarkably higher potency than AAV9 in Ly6a-transfected HEK293T cells. Representative images are shown. Scale bars=500 μm. FIG. 9B depicts representative images of HEK cells cultured in 96-well plates, either mock-transfected (none) or transfected with a potential receptor, infected with 1×10⁹ v.g. per well of PHP.B sequence family AAVs (AAVs labeled as “Ly6a AAV” were Ly6a binding) or Ly6a-independent AAVs (labeled as Ly6a-independent AAVs) packaging CAG-mNeonGreen and imaged 24 hours post-transduction. An overlay of brightfield and fluorescence images is presented. Scale bars=200 μm. FIG. 9C depicts representative images of HEK cells cultured in 96-well plates, either mock-transfected (none) or transfected with Ly6a or Slco1c1. Slco1c1-infected cells displayed dim and diffuse fluorescence that extended beyond cell boundaries for every AAV tested. An overlay of brightfield and fluorescence images is presented. Scale bar=200 μm.

FIG. 10A depicts non-limiting exemplary embodiments and data related to the expression of selected membrane proteins from the cell culture screen confirmed by immunofluorescence. DAPI and immunofluorescence (IF) overlay for untransfected and receptor-transfected HEK293T cells stained with the indicated antibody as well as the IF channel alone from receptor-transfected HEK293T cell images (at left). Scale bars=50 μm. FIG. 10B depicts non-limiting, exemplary brightfield (left in each column) and fluorescence (right in each column) images of PHP.eB, PHP.N, and CAP-B10 packaging CAG-mNeonGreen as well as CAP-B22 packaging CAG-NLS-GFP at two different doses in mock-transfected and Ly6a-expressing cells. Ly6a-interacting AAVs engineered from PHP.eB display the same degree of potency enhancement in cell culture. Scale bar=100 μm. FIG. 10C depicts non-limiting exemplary embodiments and data related to the potency of CAP-B10 and CAP-B22. CAP-B10 and CAP-B22 did not display a potency enhancement with marmoset Ly6 proteins. The potency of engineered AAVs for HEK293T cells transfected with the potential receptors is shown. Extent of infection (Max: 0.52, Min: 0.05). Total brightness per signal area (Max: 0.79, Min: 0.17).

FIG. 11A depicts non-limiting exemplary embodiments and data related to the performance of PHP.N, AAV9 and PHP.eB. In CBA/J mice at high doses, PHP.N outperformed AAV9 and PHP.eB. Animals were retro-orbitally injected at 6-8 weeks old with 1×10¹² v.g. of AAV9, PHP.eB, or PHP.N packaging CAG-mNeonGreen (n=3). Four weeks after injection, tissues were collected and imaged to detect virus potency. Each data point in the thalamus mean pixel intensity quantitation represents one animal and bars indicate the mean values. FIG. 11B depicts non-limiting exemplary embodiments and data related to the quantification of AAV potency in WT and Car4-KO mice. The mean pixel intensities of whole brain sagittal slices and liver sections were quantified. Each individual data point was then normalized to the mean PHP.eB intensity. Each data point represents one animal and bars indicate the mean values.

FIG. 12 depicts non-limiting exemplary embodiments and data related to enriched capsid variants during M-CREATE selections. Capsids were ranked according to their enrichment scores from round 1 and round 2 selections using M-CREATE Cre-dependent recovery or Cre-independent recovery. Sequence frequency plots indicate the patterns of the most enriched variants from each round and selection type.

FIG. 13A-FIG. 13D depict non-limiting exemplary embodiments and data related to snapshots of PHP.eB-Ly6a interaction yielded by an integrative structure modeling method. FIG. 13A depicts a workflow for modeling engineered AAV-receptor complex structures (PHP.eB: colored by pLDDT score, Ly6a: indicated by arrows). FIG. 13B depicts a comparison between computationally modeled PHP.eB-Ly6a and CryoEM structure of unbound PHP.eB (PDB ID: 7WQO). All high-confidence residues (pLDDT>45, see left panel arrow) within the inserted peptide showed consistent conformations between the two models except for F6′, which did not have clear side chain density in the CryoEM map. The side chain of F6′ predicted in the unbound PHP.eB model would cause a steric clash in the predicted complex model. FIG. 13C depicts assembled PHP.eB capsid-Ly6a model representing an ensemble of all Ly6a binding states, despite steric clashes, as would occur in cryo-EM particle reconstruction (PHP.eB: colored by pLDDT score, Ly6a: indicated by arrow). Top: cross section of the assembled capsid model. Middle: zoom-in of a 3-fold spike, highlighting steric clashes between three different binding states. FIG. 13D depicts overlaid structures of computationally modeled PHP.eB-Ly6a (PHP.eB: rainbow) and CryoEM model of PHP.eB-AAVR PKD2 (PDB ID: 7WQP).

FIG. 14A-FIG. 14B depict non-limiting exemplary embodiments and data related to modeling peptide complexes with Ly6c1 and mouse Car4. FIG. 14A depicts a comparison between the computationally modeled PHP.C2-Ly6c1 binding poses predicted by AlphaFold-Multimer v1 and v2. PHP.C2 peptides were colored by residue according to pLDDT score. Ly6c1 residues chosen for mutation are shown as spheres. The potency of PHP.C2 for HEK293T cells transfected with wild type or mutant Ly6c1 receptors is also shown (FIG. 14A (Continued)). Extent of infection (Max: 0.67, Min: 0.13). Total brightness per signal area (Max: 0.72, Min: 0.31). FIG. 14B depicts the top five AlphaFold2-predicted mouse Car4-9P31 and Car4-9P36 peptide complex structures, with 9P31 and 9P36 peptides colored by pLDDT score at each residue, and potency of 9P31 and 9P36 for HEK293T cells transfected with wild type or mutant Car4 receptors designed to disrupt putative site 2 binding. Extent of infection (top, Max: 0.99, Min: 0.12, bottom, Max: 0.67, Min: 0.13). Total brightness per signal area (top, Max: 0.89, Min: 0.33, bottom, Max: 0.72, Min: 0.31).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for increasing permeability of the blood brain barrier. In some embodiments, the method comprises providing a targeting peptide capable of binding to a carbonic anhydrase IV, thereby increasing permeability of the blood brain barrier.

Disclosed herein include methods for increasing permeability of the blood brain barrier. In some embodiments, the method comprises reducing carbonic anhydrase IV activity, thereby increasing permeability of the blood brain barrier.

Disclosed herein include methods for delivering a payload to a nervous system. In some embodiments, the method comprises providing a targeting peptide capable of binding to a carbonic anhydrase IV or a derivative thereof, wherein the targeting peptide is part of a delivery system, and wherein the delivery system comprises a nucleic acid sequence to be delivered to a nervous system, and administering the delivery system to the subject. In some embodiments, the delivery system is an AAV vector. Disclosed herein also include recombinant AAV (rAAV) vectors comprising a targeting peptide having a binding specificity to a carbonic anhydrase IV.

Disclosed herein include antibodies or fragments thereof. In some embodiments, the antibody or fragment thereof comprises a peptide or a portion thereof having specificity to a carbonic anhydrase IV. The antibody or fragment thereof can comprise (1) an amino acid sequence selected from SEQ ID NOs: 11-12 and SEQ ID NOs: 70-174; (2) an amino acid sequence having at least 80% sequence identity to an amino acid sequence selected SEQ ID NOs: 11-12 and SEQ ID NOs: 70-174. Disclosed herein include antibody conjugates comprising the antibody or fragment thereof disclosed herein. In some embodiments, the antibody conjugate can further comprise a therapeutic agent or a detectable label. Disclosed herein include peptides or derivatives or conjugates thereof, having specificity to a carbonic anhydrase IV. Disclosed herein include nucleic acids, comprising a sequence encoding the antibody or fragment thereof or the peptide or the derivative or the conjugate thereof disclosed herein.

Disclosed herein include delivery systems comprising (1) a targeting peptide having specificity to a carbonic anhydrase IV; and (2) a pharmaceutical agent.

Disclosed herein include methods for designing a targeting peptide having specificity to a carbonic anhydrase IV. In some embodiments, the method comprises: generating in silico one or more targeting peptides each capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179: or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180.

Disclosed herein include methods for increasing permeability of the blood brain barrier in a murine species. In some embodiments, the method comprises: providing a targeting peptide capable of binding to Ly6c1, thereby increasing permeability of the blood brain barrier in the murine species.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see e.g., Lewin, “Genes V” (Oxford University Press, Oxford) pages 847-873). Exemplary regulatory elements in prokaryotes include promoters, operator sequences and a ribosome binding site. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, the term “variant” can refer to a polynucleotide or polypeptide having a sequence substantially similar to a reference polynucleotide or polypeptide. In the case of a polynucleotide, a variant can have deletions, substitutions, and/or additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

As used herein, the term “binding” refers to a non-covalent interaction between macromolecules (e.g., between two proteins and/or peptides). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

The wording “specific,” “specifically,” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred. The term “specific,” “specifically,” or “specificity” as used herein with reference to a binding of a targeting peptide to carbonic anhydrase IV refers to the ability of the targeting peptide to form stable complex with carbonic anhydrase IV, with substantially less to no binding to macromolecules other than carbonic anhydrase IV that may be present. The term “specific,” “specifically,” or “specificity” as used herein with reference to a binding of a targeting peptide to carbonic anhydrase IV also refers to the ability of carbonic anhydrase IV to form stable complex with the targeting peptide, with substantially less to no binding to candidate peptides other than the identified targeting peptide that may be present.

The term “AAV” or “adeno-associated virus” refers to a Dependoparvovirus within the Parvoviridae genus of viruses. For example, the AAV can be an AAV derived from a naturally occurring “wild-type” virus, an AAV derived from an rAAV genome packaged into a capsid derived from capsid proteins encoded by a naturally occurring cap gene and/or an rAAV genome packaged into a capsid derived from capsid proteins encoded by a non-natural capsid cap gene. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infects primates. Likewise, an AAV may infect bovine animals (e.g., “bovine AAV”, and the like). In some instances, the AAV is wild type, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be an rAAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV (ITR sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

The terms “native” and “wild type” are used interchangeably herein, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus, the term “antibody” includes any protein or peptide containing molecule that comprises at least a portion of an immunoglobulin molecule having biological activity of binding to the antigen. Examples of such include, but are not limited to, a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The blood-brain barrier (BBB) presents a major challenge to delivering large molecules to the central nervous system (CNS). This is due in part to the scarcity of effective targets for BBB-crossing, the identification of which is the crucial first step of drug development.

Disclosed herein include novel receptors on the BBB interface for enhancing BBB crossing (e.g., carbonic anhydrase IV), and related methods of using the same to increase the permeability of the BBB and to deliver an agent to a nervous system (e.g., CNS). Disclosed herein also include targeting peptides or derivatives thereof capable of binding to a BBB crossing receptor disclosed herein (e.g., carbonic anhydrase IV), and recombinant AAV vectors, antibodies or fragments thereof, peptides or derivatives thereof, which comprise the targeting peptides or derivatives thereof. Disclosed herein also includes methods of designing targeting peptides having binding specificity to a carbonic anhydrase IV. The identification of carbonic anhydrase IV and structural insights from computational modeling provide new paths toward human brain-penetrant chemicals (drugs) and biologicals (including gene delivery).

### Receptors for Enhanced Blood-Brain Barrier Crossing

Blood-brain barrier (BBB) has emerged as a complex, dynamic, adaptable interface that controls the exchange of substances between the central nervous system (CNS) and the blood, to prevent the uncontrolled leakage of substances from the blood into the brain. The cells that make up the structure of the BBB include mostly brain endothelial cells, which constantly communicate with the other cells of the CNS (e.g., astrocytes, microglia, neurons, mast cells and pericytes, as well as circulating immune cells), adapting their behaviors to serve the needs of the CNS, responding to pathological conditions, and in some cases participating in the onset, maintenance or progression of disease. The complexity of BBB functions explains much of the difficulty in developing drugs that can cross the BBB. Utilizing receptors on the BBB interface can offer a method of crossing BBB.

Disclosed herein includes novel receptors on the BBB interface and methods of using the same to enhance BBB crossing and CNS potency, such as increasing the permeability of the BBB and delivering a therapeutic agent across the BBB to a nervous system. In some embodiments, the receptor for enhancing BBB crossing is carbonic anhydrase IV. In some embodiments, the BBB-crossing receptor is Ly6c1. Without being bound by any theory, the novel target receptors disclosed herein may facilitate enhanced BBB receptor-mediated transcytosis across various species, including mammals such as human.

In some embodiments, a method of increasing permeability of the BBB comprises providing a targeting peptide capable of binding to a BBB crossing receptor (e.g., carbonic anhydrase IV or lymphocyte antigen 6 complex, locus C1 (Ly6c1)), thereby increasing permeability of the BBB (e.g., through transcytosis). In some embodiments, at least one activity of the BBB-crossing receptor (e.g., carbonic anhydrase IV or Ly6c1) can be reduced through binding to a targeting peptide. Accordingly, in some embodiments, a method of increasing permeability of the BBB comprises reducing the activity of carbonic anhydrase IV or Ly6c1, thereby increasing permeability of the BBB. In some embodiments, a targeting peptide binds to one or more of the zinc binding site (e.g., a catalytic pocket) and substrate binding site of the carbonic anhydrase IV. The carbonic anhydrase IV can be a vertebrate carbonic anhydrase IV including non-human primates and humans. In some embodiments, the carbonic anhydrase IV is a mouse carbonic anhydrase IV (Car4), a human carbonic anhydrase IV (CA4), or a variant or a homolog thereof. In some embodiments, a targeting peptide binds to Ly6c1 in a murine species, thereby increasing permeability of the blood brain barrier in the murine species.

Carbonic Anhydrase IV

In some embodiments, the BBB-crossing receptor disclosed herein capable of facilitating the delivery of a pharmaceutical agent across the BBB is a carbonic anhydrase IV. Carbonic anhydrase IV is an isozyme that belongs to the carbonic anhydrase family, a family of zinc metalloenzymes, which catalyzes the reversible reaction of hydration of CO₂ (H₂O+CO₂⇄HCO₃⁻+H⁺), allowing the enzyme to regulate intra- and extra-cellular concentrations of CO₂, H⁺, and HCO₃⁻. The carbonic anhydrases participate in a variety of biological processes, including respiration, calcification, acid-base balance, bone resorption, and the formation of aqueous humor, cerebrospinal fluid, saliva, and gastric acid. The carbonic anhydrases show extensive diversity in tissue distribution and in their subcellular localization. There are at least seven genetically distinct isozymes of mammalian carbonic anhydrase, designated I-VII, each of which catalyzes the reversible hydration of carbon dioxide by a zinc-hydroxide mechanism. Physiological functions that are regulated by carbonic anhydrase comprise, for example, removal of HCO³⁻ in lung by respiration, reutilization of HCO³⁻ in kidney, production of aqueous humor in eyes, cerebrospinal fluids in brain, gastric juice production in stomach, pancreatic juice, and bone resorption by osteoclasts. Carbonic anhydrase family members also play important roles in metabolic processes that include ureagenesis, gluconeogenesis, and lipogenesis.

Different from other carbonic anhydrases that are either soluble or attached to the plasma membrane by a membrane-spanning domain, carbonic anhydrase IV is a glycosylphosphatidyl-inositol-anchored membrane isozyme. Carbonic anhydrase IV is broadly conserved across vertebrates and has similar CNS expression profiles in humans, with a recent single cell analysis of human brain vasculature confirming CA4's expression in the human BBB. Carbonic anhydrase IV has been shown to regulate pH, which is associated with neural discharge and can influence neuronal function through ion-gated channels.

In some embodiments, the carbonic anhydrase IV disclosed herein is a human carbonic anhydrase IV (CA4). CA4 is known to localize on the luminal surface of brain endothelial cells throughout the cortex and cerebellum where it enzymatically modulates carbon dioxide-bicarbonate balance. Human CA4 has been previously characterized as a 35-kDa protein with a “high activity” in CO₂ hydration and a higher activity than other isozymes in catalyzing the dehydration of HCO₃⁻. In general, human CA4 contains an 18-amino acid signal sequence at the N-terminal of the protein for endoplasmic reticulum (ER) translocation and a 260-amino acid “CA domain” containing active site amino acid residues that shows 30-36% homology with cytoplasmic CAs. At the C-terminal, an additional 27 amino acid residues containing the hydrophobic sequences of 21 amino acids sufficient to span the membrane are preceded by the 6-amino acid signal sequence for GPI-anchoring. The amino acid residue, Ser 266, was identified as the site for the attachment of the GPI anchor. The removal of C-terminal hydrophobic domain found in the CA4 precursor has important impact on GPI-anchoring, cell surface expression, and realization of the enzyme activity. Based on amino acid sequences deduced from the nucleotide sequence, human CA4 contains no classical consensus sites (Asn-Xxx-Ser/Thr) for N-glycosylation. Human CA4 also contains no oligosaccharide chains, while other mammalian carbonic anhydrase IV (e.g. mouse carbonic anhydrases IV (Car4)) are glycoproteins with one to several oligosaccharide side chains.

In some embodiments, the carbonic anhydrase IV disclosed herein is a mouse carbonic anhydrase IV (Car4). Car4 has recently been found to be among the mouse proteins most strongly positively correlated with plasma-protein uptake in the brain (slightly stronger than the often-targeted transferrin receptor). This property is useful for identifying receptors for enhanced BBB crossing. Car4 is also expressed in the GI tract, kidney, and lung, as well as taste receptor cells where it allows the sensing of carbonation. Mouse Car4 and human CA4 are highly homologous, containing the same amino acids at positions crucial for enzyme activity (e.g., histidine residue 64 (His 64)), with several differences including, for example, that mouse Car4 is an N-linked glycoprotein and the CO₂ hydration rate catalyzed by mouse Car4 is much lower than human CA4. Without being bound by any theory, the lower enzyme activity of mouse Car4 may be associated with the replacement of Gly 63 in human CA4 with Gln 63, among several other amino acid replacements. Another difference between mouse Car4 and human CA4 is the Val-131-Asp-136 segment (130's segment) that forms an α-helix in mouse Car4 and an extended loop in human CA4.

In some embodiments, a carbonic anhydrase IV (Car4) disclosed herein as a receptor for enhancing BBB crossing can be any carbonic anhydrase IV, such as a mouse Car4, a human CA4, or a homology or a variant thereof. Carbonic anhydrase IV homologs and/or variants can be derived from a vertebrate species including, but not limited to, mouse, rat, human, bovine, rabbit, monkey, pig, horse, rainbow trout, chimpanzee, squirrel, chicken, goat, and sheep. Carbonic anhydrase IV homologs from various species can be found in public databases identifiable to a person skilled in the art, including for example UniProt, NCBI, and Swiss-Prot.

In some embodiments, a mouse Car4 disclosed herein as a receptor for enhancing BBB crossing comprises a sequence having SEQ ID NO: 179. In some embodiments, a human CA4 disclosed herein as a receptor for enhancing BBB crossing comprises a sequence having SEQ ID NO: 180. In some embodiments, a receptor disclosed herein for enhancing BBB crossing is a carbonic anhydrase IV homolog or variant of human CA4 (e.g., SEQ ID NO: 180) or mouse Car4 (SEQ ID NO: 179). The carbonic anhydrase IV homologs and variants can be about or can be at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of SEQ ID NO: 179 or SEQ ID NO: 180.

In some embodiments, a targeting peptide can bind to a carbonic anhydrase IV disclosed herein (e.g., mouse Car4, human CA4 or a homology or a variant thereof), thereby increasing permeability of the BBB (e.g., through transcytosis). In some embodiments, the increase in the permeability of the BBB is achieved by altering (e.g., increasing or decreasing) the carbonic anhydrase IV activity, such as reducing its activity.

In some embodiments, the alteration of carbonic anhydrase IV activity is achieved by a targeting peptide binding to one or more active sites of the carbonic anhydrase IV including the zinc binding site and the hydrophobic substrate binding pocket. For example, the targeting peptide can bind to the zinc binding site, the hydrophobic substrate binding pocket, or both.

The zinc binding site in carbonic anhydrase IV has a conserved structure dominated by a β-sheet super-structure with a metal binding site formed by at least three His residues. Without being bound by any theory, it is believed that the zinc binding site is on one face of the β-sheet at the bottom of a 15-Å-deep, conical active site cleft in which zinc is liganded by three His residues and hydroxide ion with tetrahedral geometry. The hydrophobic substrate binding pocket is adjacent to zinc-bound hydroxide, formed in large part by bulky residues such as Val at its base and Val, Trp and Leu at its neck. This pocket is highly conserved among all active isozymes on the basis of phylogenetic comparisons. Without being bound by any theory, it is believed that the hydrophobic pocket has a minimum width and depth for efficient catalysis, and linear free energy relationships indicate that the volume of the amino acid residue at the base of the pocket and the hydrophobicity of residues at the neck of the pocket are critical for activity. Both the zinc binding site and the hydrophobic substrate binding pocket are highly conserved among carbonic anhydrase isozymes.

A targeting peptide can bind to one or more conservative amino acid residues at the interaction sites of the carbonic anhydrase IV. In some embodiments, computational modeling methods (e.g., AlphaFolder2-multimer) can be used to predict peptide interaction sites (e.g., AAV interaction sites when the peptide is part of an AAV capsid) on carbonic anhydrase IV (e.g., mouse Car4). Table 1 provides a list of Alphafold2-multimer predicted AAV-interaction site on mouse carbonic anhydrase IV and the corresponding residues in human CA4.

TABLE 1
Exemplary AAV-interaction sites on mouse and human carbonic
anhydrase IV predicted by AlphaFold2-multimer

| | | Number of | Corresponding |
|---|---|---|---|
| Mouse Car4 | | contacting atoms | human CA4 |
| (U37091.1) | | (<5 angstrom, | (NM_000717.5) |
| residue index | pLDDT | non- hydrogen) | residue index |
| S20 | 59.17 | 5 | S21 |
| G21 | 82.35 | 4 | H22 |
| W22 | 90.49 | 12 | W23 |
| L36 | 91.66 | 6 | L37 |
| W41 | 93.24 | 2 | W42 |
| P42 | 90.06 | 2 | G43 |
| E90 | 94.79 | 4 | M92 |
| V111 | 93.54 | 3 | K113 |
| Q112 | 94.05 | 4 | Q114 |
| H114 | 96.91 | 4 | H116 |
| H139 | 98.17 | 4 | H141 |
| V141 | 95.98 | 3 | V143 |
| K143 | 92.39 | 5 | E145 |
| F156 | 93.28 | 4 | Q158 |
| L217 | 94.14 | 6 | L224 |
| T218 | 95.56 | 2 | T225 |
| T219 | 93.34 | 6 | T226 |
| P220 | 89.71 | 6 | P227 |
| N221 | 87.81 | 6 | T228 |
| D223 | 90.74 | 3 | D231 |
| W228 | 98.7 | 2 | W236 |

In some embodiments, the targeting peptide can interact with one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179. In some embodiments, the target peptide can interact with one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180.

Lymphocyte Antigen 6

In some embodiments, the BBB-crossing receptor disclosed herein capable of enhancing BBB crossing upon binding to a targeting peptide in a murine species is a lymphocyte antigen 6 complex, locus C1 (Ly6c1). Ly6c1 is a glycosylphosphatidyl-inositol-anchored (GPI-anchored) protein and belongs to the Ly6 family of GPI-anchored surface glycoproteins. Ly6 known as lymphocyte antigen 6 or urokinase-type plasminogen activator receptor is a family of closely related murine glycoproteins that share a common structure but differ in their tissue expression patterns and function. Ly6 family proteins are structurally related proteins characterized by the LU domain, an 80 amino acid domain containing ten cysteines arranged in a specific spacing pattern that allows distinct disulfide bridges which create the three-fingered structural motif. Ly6c1 can be used as a novel receptor, serving as potent research tools in mice, including for example designing enhanced AAV capsids against Ly6c1 using directed evolution in combination with in silico design.

In some embodiments, a Ly6c1 receptor disclosed herein as a receptor for enhanced BBB crossing can be a mouse Ly6c1 or a homology or a variant thereof. In some embodiments, the Ly6c1 receptor from Mus musculus has an amino acid sequence of SEQ ID NO 293 (Uniprot Accession: P0CW02). In some embodiments, In some embodiments, a Ly6c1 receptor disclosed herein for enhancing BBB crossing can be about or can be at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of SEQ ID NO: 293.

In some embodiments, a targeting peptide can bind to a Ly6c1 disclosed herein, thereby increasing permeability of the BBB in a murine species. A murine species can be selected from the species in a Muridae family, Murinae subfamily, Mus genus, and/or Mus subgenus.

### Targeting Peptides and in Silico Screening

The methods of using the receptors described herein to increase the permeability of the BBB and to deliver a pharmaceutical agent across the BBB to a nervous system comprise providing a targeting peptide capable of binding to a BBB crossing receptor disclosed herein (e.g., Ly6c1 or carbonic anhydrase IV) or a targeting peptide having a binding specificity to the BBB crossing receptor (e.g., Ly6c1 or carbonic anhydrase IV such as Car4 and CA4).

Targeting Peptides for Carbonic Anhydrase IV

In some embodiments, the method comprises providing a targeting peptide capable of binding to a carbonic anhydrase IV or a targeting peptide having a binding specificity to the carbonic anhydrase IV (e.g., Car4 and CA4) to a subject.

The targeting peptide can bind to the zinc binding site and/or the hydrophobic substrate binding pocket of the carbonic anhydrase IV as described above, thereby altering the activity of carbonic anhydrase IV (e.g., reducing the carbonic anhydrase IV activity). The length of the targeting peptide can be about, at least or at most 4-15 (e.g., 4, 5, 6, 7, 8, 9, 10, 11, 12, 13, 14, or 15) amino acids in length.

The targeting peptide can comprise (1) an amino acid sequence selected from SEQ ID NOs: 11-12 and SEQ ID NOs: 70-174; (2) an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from SEQ ID NOs: 11-12 and SEQ ID NOs: 70-174.

The targeting peptide can comprise an amino acid sequence selected from SEQ ID NOs: 70-108. The targeting peptide can comprise an amino acid sequence selected from SEQ ID NOs: 70-86. The targeting peptide can comprise an amino acid sequence selected from AKPTPLLGLLQAQTG (SEQ ID NO: 70), AKPTPLLLLLQAQTG (SEQ ID NO: 71), AQPPLGGLLAQAQTG (SEQ ID NO: 72), AKPPGPWAEAQAQTG (SEQ ID NO: 73), or AQPPLLGGLAQAQTG (SEQ ID NO: 74). In some embodiments, the targeting peptide comprises an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from SEQ ID NOs: 70-74.

The targeting peptide can comprise at least 4 contiguous amino acids from the sequence AKPTPLLGLLQAQTG (SEQ ID NO: 70). For example, the targeting peptide can comprise the sequence of KPTP (SEQ ID NO: 181), PTPL (SEQ ID NO: 182), TPLL (SEQ ID NO: 183), PLLG (SEQ ID NO: 184), LLGL (SEQ ID NO: 185), LGLL (SEQ ID NO: 186) or GLLQ (SEQ ID NO: 187). The targeting peptide can comprise at least 5 contiguous amino acids from the sequence of AKPTPLLGLLQAQTG (SEQ ID NO: 70). For example, the targeting peptide can comprise the sequence of KPTPL (SEQ ID NO: 188), PTPLL (SEQ ID NO: 189), TPLLG (SEQ ID NO: 190), PLLGL (SEQ ID NO: 191), LLGLL (SEQ ID NO: 192) or LGLLQ (SEQ ID NO: 193). The targeting peptide can comprise at least 6 contiguous amino acids from the sequence of AKPTPLLGLLQAQTG (SEQ ID NO: 70). For example, the targeting peptide can comprise the sequence of KPTPLL (SEQ ID NO: 194), PTPLLG (SEQ ID NO: 195), TPLLGL (SEQ ID NO: 196), PLLGLL (SEQ ID NO: 197) or LLGLLQ (SEQ ID NO: 198). The targeting peptide can comprise at least 7 contiguous amino acids from the sequence of AKPTPLLGLLQAQTG (SEQ ID NO: 70). For example, the targeting peptide can comprise the sequence of KPTPLLG (SEQ ID NO: 199), PTPLLGL (SEQ ID NO: 200), TPLLGLL (SEQ ID NO: 201) or PLLGLLQ (SEQ ID NO: 202). The targeting peptide can comprise at least 8 contiguous amino acids from the sequence of AKPTPLLGLLQAQTG (SEQ ID NO: 70). For example, the targeting peptide can comprise the sequence of KPTPLLGL (SEQ ID NO: 203), PTPLLGLL (SEQ ID NO: 204), or TPLLGLLQ (SEQ ID NO: 205). The targeting peptide can comprise at least 9 contiguous amino acids from the sequence of AKPTPLLGLLQAQTG (SEQ ID NO: 70). For example, the targeting peptide can comprise the sequence of KPTPLLGLL (SEQ ID NO: 206), or PTPLLGLLQ (SEQ ID NO: 207). In some embodiments, the targeting peptide comprises AKPTPLLGLLQAQTG (SEQ ID NO: 70).

The targeting peptide can comprise at least 4 contiguous amino acids from the sequence AKPTPLLLLLQAQTG (SEQ ID NO: 71). For example, the targeting peptide can comprise the sequence of KPTP (SEQ ID NO: 181), PTPL (SEQ ID NO: 182), TPLL (SEQ ID NO: 183), PLLL (SEQ ID NO: 208), LLLL (SEQ ID NO: 209), or LLLQ (SEQ ID NO: 210). The targeting peptide can comprise at least 5 contiguous amino acids from the sequence of AKPTPLLLLLQAQTG (SEQ ID NO: 71). For example, the targeting peptide can comprise the sequence of KPTPL (SEQ ID NO: 188), PTPLL (SEQ ID NO: 189), TPLLL (SEQ ID NO: 211), PLLLL (SEQ ID NO: 212), LLLLL (SEQ ID NO: 213) or LLLLQ (SEQ ID NO: 214). The targeting peptide can comprise at least 6 contiguous amino acids from the sequence of AKPTPLLLLLQAQTG (SEQ ID NO: 71). For example, the targeting peptide can comprise the sequence of KPTPLL (SEQ ID NO: 194), PTPLLL (SEQ ID NO: 215), TPLLLL (SEQ ID NO: 216), PLLLLL (SEQ ID NO: 217) or LLLLLQ (SEQ ID NO: 218). The targeting peptide can comprise at least 7 contiguous amino acids from the sequence of AKPTPLLLLLQAQTG (SEQ ID NO: 71). For example, the targeting peptide can comprise the sequence of KPTPLLL (SEQ ID NO: 219), PTPLLLL (SEQ ID NO: 220), TPLLLLL (SEQ ID NO: 221) or PLLLLLQ (SEQ ID NO: 222). The targeting peptide can comprise at least 8 contiguous amino acids from the sequence of AKPTPLLLLLQAQTG (SEQ ID NO: 71). For example, the targeting peptide can comprise the sequence of KPTPLLLL (SEQ ID NO: 223), PTPLLLLL (SEQ ID NO: 224), or TPLLLLLQ (SEQ ID NO: 225). The targeting peptide can comprise at least 9 contiguous amino acids from the sequence of AKPTPLLLLLQAQTG (SEQ ID NO: 71). For example, the targeting peptide can comprise the sequence of KPTPLLLLL (SEQ ID NO: 226), or PTPLLLLLQ (SEQ ID NO: 227). In some embodiments, the targeting peptide comprises AKPTPLLLLLQAQTG (SEQ ID NO: 71).

The targeting peptide can comprise at least 4 contiguous amino acids from the sequence AQPPLGGLLAQAQTG (SEQ ID NO: 72). For example, the targeting peptide can comprise the sequence of PPLG (SEQ ID NO: 228), PLGG (SEQ ID NO: 229), LGGL (SEQ ID NO: 230), GGLL (SEQ ID NO: 231), GLLA (SEQ ID NO: 232) or LLAQ (SEQ ID NO: 233). The targeting peptide can comprise at least 5 contiguous amino acids from the sequence of AQPPLGGLLAQAQTG (SEQ ID NO: 72). For example, the targeting peptide can comprise the sequence of PPLGG (SEQ ID NO: 234), PLGGL (SEQ ID NO: 235), LGGLL (SEQ ID NO: 236), GGLLA (SEQ ID NO: 237) or GLLAQ (SEQ ID NO: 238). The targeting peptide can comprise at least 6 contiguous amino acids from the sequence of AQPPLGGLLAQAQTG (SEQ ID NO: 72). For example, the targeting peptide can comprise the sequence of PPLGGL (SEQ ID NO: 239), PLGGLL (SEQ ID NO: 240), LGGLLA (SEQ ID NO: 241), or GGLLAQ (SEQ ID NO: 242). The targeting peptide can comprise at least 7 contiguous amino acids from the sequence of AQPPLGGLLAQAQTG (SEQ ID NO: 72). For example, the targeting peptide can comprise the sequence of PPLGGLL (SEQ ID NO: 243), PLGGLLA (SEQ ID NO: 244), or LGGLLAQ (SEQ ID NO: 245). The targeting peptide can comprise at least 8 contiguous amino acids from the sequence of AQPPLGGLLAQAQTG (SEQ ID NO: 72). For example, the targeting peptide can comprise the sequence of PPLGGLLA (SEQ ID NO: 246), or PLGGLLAQ (SEQ ID NO: 247). In some embodiments, the targeting peptide comprises AQPPLGGLLAQAQTG (SEQ ID NO: 72).

The targeting peptide can comprise at least 4 contiguous amino acids from the sequence AKPPGPWAEAQAQTG (SEQ ID NO: 73). For example, the targeting peptide can comprise the sequence of KPPG (SEQ ID NO: 248), PPGP (SEQ ID NO: 249), PGPW (SEQ ID NO: 250), GPWA (SEQ ID NO: 251), PWAE (SEQ ID NO: 252), WAEA (SEQ ID NO: 253) or AEAQ (SEQ ID NO: 254). The targeting peptide can comprise at least 5 contiguous amino acids from the sequence of AKPPGPWAEAQAQTG (SEQ ID NO: 73). For example, the targeting peptide can comprise the sequence of KPPGP (SEQ ID NO: 255), PPGPW (SEQ ID NO: 256), PGPWA (SEQ ID NO: 257), GPWAE (SEQ ID NO: 258), PWAEA (SEQ ID NO: 259) or WAEAQ (SEQ ID NO: 260). The targeting peptide can comprise at least 6 contiguous amino acids from the sequence of AKPPGPWAEAQAQTG (SEQ ID NO: 73). For example, the targeting peptide can comprise the sequence of KPPGPW (SEQ ID NO: 261), PPGPWA (SEQ ID NO: 262), PGPWAE (SEQ ID NO: 263), GPWAEA (SEQ ID NO: 264) or PWAEAQ (SEQ ID NO: 265). The targeting peptide can comprise at least 7 contiguous amino acids from the sequence of AKPPGPWAEAQAQTG (SEQ ID NO: 73). For example, the targeting peptide can comprise the sequence of KPPGPWA (SEQ ID NO: 266), PPGPWAE (SEQ ID NO: 267), PGPWAEA (SEQ ID NO: 268) or GPWAEAQ (SEQ ID NO: 269). The targeting peptide can comprise at least 8 contiguous amino acids from the sequence of AKPPGPWAEAQAQTG (SEQ ID NO: 73). For example, the targeting peptide can comprise the sequence of KPPGPWAE (SEQ ID NO: 270), PPGPWAEA (SEQ ID NO: 271), or PGPWAEAQ (SEQ ID NO: 272). The targeting peptide can comprise at least 9 contiguous amino acids from the sequence of AKPPGPWAEAQAQTG (SEQ ID NO: 73). For example, the targeting peptide can comprise the sequence of KPPGPWAEA (SEQ ID NO: 273), or PPGPWAEAQ (SEQ ID NO: 274). In some embodiments, the targeting peptide comprises AKPPGPWAEAQAQTG (SEQ ID NO: 73).

The targeting peptide can comprise at least 4 contiguous amino acids from the sequence AQPPLLGGLAQAQTG (SEQ ID NO: 74). For example, the targeting peptide can comprise the sequence of PPLL (SEQ ID NO: 275), PLLG (SEQ ID NO: 184), LLGG (SEQ ID NO: 276), LGGL (SEQ ID NO: 230), GGLA (SEQ ID NO: 277) or GLAQ (SEQ ID NO: 278). The targeting peptide can comprise at least 5 contiguous amino acids from the sequence of AQPPLLGGLAQAQTG (SEQ ID NO: 74). For example, the targeting peptide can comprise the sequence of PPLLG (SEQ ID NO: 279), PLLGG (SEQ ID NO: 280), LLGGL (SEQ ID NO: 281), LGGLA (SEQ ID NO: 282), or GGLAQ (SEQ ID NO: 283). The targeting peptide can comprise at least 6 contiguous amino acids from the sequence of AQPPLLGGLAQAQTG (SEQ ID NO: 74). For example, the targeting peptide can comprise the sequence of PPLLGG (SEQ ID NO: 284), PLLGGL (SEQ ID NO: 285), LLGGLA (SEQ ID NO: 286), or LGGLAQ (SEQ ID NO: 287). The targeting peptide can comprise at least 7 contiguous amino acids from the sequence of AQPPLLGGLAQAQTG (SEQ ID NO: 74). For example, the targeting peptide can comprise the sequence of PPLLGGL (SEQ ID NO: 288), PLLGGLA (SEQ ID NO: 289), or LLGGLAQ (SEQ ID NO: 290). The targeting peptide can comprise at least 8 contiguous amino acids from the sequence of AQPPLLGGLAQAQTG (SEQ ID NO: 74). For example, the targeting peptide can comprise the sequence of PPLLGGLA (SEQ ID NO: 291), or PLLGGLAQ (SEQ ID NO: 292). In some embodiments, the targeting peptide comprises AQPPLLGGLAQAQTG (SEQ ID NO: 74).

Targeting Peptides for Ly6c1

In some embodiments, the method comprises providing a targeting peptide capable of binding to a Ly6c1 or a targeting peptide having a binding specificity to the Ly6c1 in a subject (e.g., mouse).

The targeting peptide can comprise 1) an amino acid sequence selected from AQFVVGQSYAQ (SEQ ID NO: 6), AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), AQWPTSYDAAQ (SEQ ID NO: 11), DGSSSYYDAAQ (SEQ ID NO: 12), AQGENPGRWAQ (SEQ ID NO: 13), DGTGQVTGWAQ (SEQ ID NO: 14), DGTGSTTGWAQ (SEQ ID NO: 15), and SPRYKGDSVAQ (SEQ ID NO: 57); or (2) an amino acid sequence having at least 80% sequence identity to an amino acid sequence selected from AQFVVGQSYAQ (SEQ ID NO: 6), AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), AQWPTSYDAAQ (SEQ ID NO: II), DGSSSYYDAAQ (SEQ ID NO: 12), AQGENPGRWAQ (SEQ ID NO: 13), DGTGQVTGWAQ (SEQ ID NO: 14), DGTGSTTGWAQ (SEQ ID NO: 15), and SPRYKGDSVAQ (SEQ ID NO: 57).

In some embodiments, the targeting peptide comprises an amino acid sequence of SPRYKGDSVAQ (SEQ ID NO: 57) or an amino acid sequence having at least (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to SPRYKGDSVAQ (SEQ ID NO: 57).

In Silico Screening of Targeting Peptides

Disclosed herein include methods for designing a targeting peptide having a binding specificity to a carbonic anhydrase IV. In some embodiments, the method comprises generating in silico one or more targeting peptides each capable of interacting with (1) one or more positions functionally equivalent to 520, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179, or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180.

In some embodiments, generating in silico the one or more targeting peptides can comprise generating in silico a plurality of candidate peptides, performing computer-assisted docking simulations for each of the plurality of candidate peptides binding to the carbonic anhydrase IV, and analyzing the molecular structure of the carbonic anhydrase IV binding to one or more of the plurality of candidate peptides to identify one or more targeting peptides capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179, or (2) one or more positions functionally equivalent to S21, H22, W23, L37, W42, G43, M92, K113, Q114, H116, H141, V143, E145, Q158, L224, T225, T226, P227, T228, D231, or W236 in the carbonic anhydrase IV having an amino acid sequence of SEQ ID NO: 180.

In silico methods can offer a high throughput approach for screening a large number of candidate peptides and identifying targeting peptides with the desired specificity. In some embodiments, the candidate peptide can comprise a portion of an AAV capsid protein. In some embodiments, the candidate peptide is part of an AAV capsid protein. The method can comprise constructing one or more peptide-receptor models for each candidate peptide in complex with a carbonic anhydrase IV (e.g., performing computer-assisted docking simulations). The molecular models can be constructed for a peptide-protein complex using any rational computational peptide design and docking methods, database, programs, or algorithms described herein or known in the field. Exemplary computational moldering methods, public database, and programs, include, but are not limited to, AlphaFold (e.g., AlphaFold2, and AlphaFold-Multimer provided by DeepMind, available at github.com/deepmind/alphafold), RoseTTAFold (github.com/RosettaCommons/RoseTTAFold), AutoDock, DOCK, FlexX, GOLD, OSPREY, SCWRL, PyMol, SWISS-MODEL (academic.oup.com/nar/article/46/Wl/W296/5000024), Protein Data Bank (PDB) (available via websites of member organizations, e.g., PDBe—pdbe.org, PDBj—pdbj.org, RCSB—rcsb.org/pdb, and BMRB—bmrb.wisc.edu), Phyre2 (nature.com/articles/nprot.2015.053), and RaptorX (nature.com/articles/nprot.2012.085).

The method can further comprise evaluating features or parameters associated with interactions between a candidate peptide and the carbonic anhydrase IV using visualization software such as PyMol, Qlucore Omics Explorer, WebMol, Insight II, Discovery Studio 2.1 and others identifiable to a person skilled in the art. Features or parameters being evaluated can comprise interface energy and physical and geometric scorings. Evaluating the features or parameters associated with interactions between a candidate peptide and the carbonic anhydrase IV can comprise measuring surface complementarity, solvent accessible surface areas, solvation free energy, electrostatic interaction energy, van der Waals energy and/or the total molecular mechanics energy. The method can also comprise determining the total number of atoms in the interface, the total number of atoms in the peptide that are clashing with the carbonic anhydrase IV, the binding angle of the peptide, and/or the binding depths of the peptide in each putative peptide-receptor complex model. The method can also comprise identifying the lowest energy conformation of a peptide-carbonic anhydrase IV complex. The energy score of each conformation can be determined by calculating the interaction energy between the peptide and carbonic anhydrase IV, including electrostatic, desolvation, van der Waals energy as will be understood by a skilled person.

In some embodiments, the targeting peptides can be assigned with a binding score and ranked based on the binding score. A threshold can be imposed to identify desired targeting peptides. The method can comprise obtaining a binding score for each of the plurality of candidate peptides binding to the carbonic anhydrase IV and selecting one or more of the plurality of candidate peptides having a binding score above a threshold value as a targeting peptide having a binding specificity (or high binding affinity) to the carbonic anhydrase IV.

A combination of physical and geometric scoring parameters including interface energy, binding angle, and binding pocket depth calculation can be used to generate a binding score. In some embodiments, the binding score for each of the plurality of candidate peptide sequences can be obtained by (1) counting a total number of atoms in the interface of a candidate peptide and the carbonic anhydrase IV; (2) counting a total number of atoms in the candidate peptide, wherein the atoms are clashing with the carbonic anhydrase IV; (3) obtaining a binding angle of the candidate peptide; and/or (4) obtaining a binding depth of the candidate peptide.

The total number of atoms in the interface of a candidate peptide and the carbonic anhydrase can be the total number of atoms within a cutoff distance between interfacing atoms of the candidate peptide and interfacing atoms of the carbonic anhydrase. The cutoff distance can vary in different embodiments. In some embodiments, the cutoff distance is about, at most, at most about 5 angstrom (e.g., 2 angstrom, 3 angstrom, 4 angstrom or 5 angstrom). The number of interfacing atoms between a candidate peptide and carbonic anhydrase IV can be different in different embodiments. For example, the number of interfacing atoms can be about or can be at least 2, 3, 4, 5, 6, 7, 8, 9, 10, 11, 12, 13, 14, 15, 16, 17, 18, 19, 20, 25, 30, 35, 40, 45, 50, or a number or a range between any of these values.

The total number of atoms in the peptide that are clashing with the carbonic anhydrase IV can be the total number of atoms within a clashing distance. A clashing distance can be defined as a distance when geometric clashes occur between peptide and receptor atoms. In some embodiments, a clashing distance can be about 1 angstrom.

The binding angle of the peptide can be defined as the angle between the vector from carbonic anhydrase IV gravity center to carbonic anhydrase IV anchor and the vector from carbonic anhydrase gravity center to peptide gravity center.

The binding depth can be defined as the difference of the distance between the closest point on the peptide to the carbonic anhydrase IV center and the minor radius of the ellipsoid hull of the carbonic anhydrase IV normalized by the minor radius.

The binding score can be the sum of a contact score calculated based on the total number of atoms in the interface of a candidate peptide and the carbonic anhydrase and the total number of clashing atoms), a binding angle, and a binding depth. In some embodiments, a binding score is equal to or greater than 0. For example, a binding score is defined as 0, if the sum of a contact score, a binding angle and a binding depth is negative. The binding score of a targeting peptide binding to carbonic anhydrase IV can be about 0, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300 or a number or a range between any two of these values.

In some embodiments, the binding score of a candidate peptide (B^{peptide}) can be defined as follows:

$${B}^{peptide} = max ( {B}_{energetic}^{peptide} + {B}_{angle}^{peptide} + {B}_{depth}^{peptide} , 0 ) \qquad ( 1 )$$

$${B}_{energetic}^{peptide} = max ( {N}_{contact}^{peptide} - {10}^{3} \cdot {N}_{clash}^{peptide} , 0 ) \qquad ( 2 )$$

$${B}_{angle}^{peptide} = \{ \begin{matrix}- {10}^{3} \cdot {( 1 - \frac{θ}{\frac{π}{2}} )}^{10} , if θ < \frac{π}{2} \\ 0 , if \frac{π}{2} <= θ <= π\end{matrix} \qquad ( 3 )$$

$${B}_{depth}^{peptide} = {10}^{2} \cdot {d}^{3} \qquad ( 4 )$$

The method can further comprise selecting one or more of the plurality of candidate peptides having a binding score above a threshold value as a targeting peptide having a binding specificity to the carbonic anhydrase IV. The threshold can be an arbitrary value defined by a user. In some embodiments, the threshold can be a binding score of a targeting peptide known for having a binding specificity to carbonic anhydrase IV. In some embodiments, results from individual pairwise competitions (a relative score between two competing peptides) can be assembled into a peptide competition metric that ranks sets of candidate peptides according to their receptor-binding probability encoded in the AlphaFold2 neural network.

Table 2 provides an exemplary list of targeting peptides ranked by Automated Pairwise Peptide Receptor Analysis for Screening Engineered AAV (APPRAISE-AAV), the details of which are described in Examples 1 and 3 of the present disclosure. B_POI of Table 2 is the binding score for each peptide of interest. Peptide sequences in Table 2 are the amino acid sequences starting with AAV9 residue A587 and ending with residue G594.

TABLE 2
APPRAISE-AAV ranked human carbonic anhydrase
IV-interacting engineered AAVs

| Rank | Peptide sequence | B_POI | SEQ ID NO: |
|---|---|---|---|
| 1 | AKPTPLLGLLQAQTG | 113.5 | 70 |
| 2 | AKPTPLLLLLQAQTG | 99.4 | 71 |
| 3 | AQPPLGGLLAQAQTG | 88.2 | 72 |
| 4 | AKPPGPWAEAQAQTG | 86.3 | 73 |
| 5 | AQPPLLGGLAQAQTG | 86.1 | 74 |
| 6 | AQPPGPWAAAQAQTG | 81.5 | 75 |
| 7 | AQPPGPYAVAQAQTG | 79.1 | 76 |
| 8 | ASSETATWATQAQTG | 79.0 | 77 |
| 9 | AQPPGPWAVAQAQTG | 76.4 | 78 |
| 10 | AQAGGDYDAVQAQTG | 76.1 | 79 |
| 11 | AQGASGIDQAQAQTG | 76.0 | 80 |
| 12 | AKPPSPWAEAQAQTG | 74.7 | 81 |
| 13 | AQGGGEYEEVQAQTG | 73.9 | 82 |
| 14 | AQAKGLYEAVQAQTG | 73.2 | 83 |
| 15 | AQPPGPWAEAQAQTG | 71.4 | 84 |
| 16 | AKPTPLLKLLQAQTG | 71.1 | 85 |
| 17 | AQAGGDYEAVQAQTG | 70.7 | 86 |
| 18 | AQPPPLGGLAQAQTG | 69.6 | 87 |
| 19 | AQPKGPWAEAQAQTG | 69.3 | 88 |
| 20 | AQAGGEYEEAQAQTG | 67.4 | 89 |
| 21 | AQGKGLYEEALAQTG | 67.0 | 90 |
| 22 | AQAKGDYEAALAQTG | 66.0 | 91 |
| 23 | AKPPSPWAKAQAQTG | 65.9 | 92 |
| 24 | AQGGGDYEAALAQTG | 63.3 | 93 |
| 25 | AQGKGDYEAALAQTG | 59.8 | 94 |
| 26 | AKPPPLLLLLQAQTG | 57.3 | 95 |
| 27 | AKPPGPFAKAQAQTG | 56.8 | 96 |
| 28 | AQPKGPYAEAQAQTG | 56.5 | 97 |
| 29 | AQPPGPYAEAQAQTG | 54.8 | 98 |
| 30 | AQGGAEVLTAQAQTG | 54.5 | 99 |
| 31 | AQGKGLYEAVQAQTG | 54.5 | 100 |
| 32 | AKPPGPWAKAQAQTG | 53.9 | 101 |
| 33 | AKPPGPFAEAQAQTG | 53.5 | 102 |
| 34 | AQAGGDYEAALAQTG | 53.1 | 103 |
| 35 | AKPPSPFAEAQAQTG | 52.3 | 104 |
| 36 | AQNKKMYYYAQAQTG | 51.4 | 105 |
| 37 | AQGGGDYEAVQAQTG | 51.2 | 106 |
| 38 | AQGKGLYEEVQAQTG | 51.1 | 107 |
| 39 | AKPPSPFAKAQAQTG | 50.7 | 108 |
| 40 | AQPPLGGGLAQAQTG | 49.5 | 109 |
| 41 | AQGGGEYEEALAQTG | 49.1 | 110 |
| 42 | AQAKGLYEAALAQTG | 47.6 | 111 |
| 43 | AQGGGEYEEAQAQTG | 46.9 | 112 |
| 44 | AQGKGGYWYGQAQTG | 46.4 | 113 |
| 45 | AQAKGLYEEAQAQTG | 46.1 | 114 |
| 46 | AQAKGLYEEVQAQTG | 45.1 | 115 |
| 47 | AQGKGLYEEAQAQTG | 45.1 | 116 |
| 48 | AQGKGDYEAVQAQTG | 43.8 | 117 |
| 49 | AQAKGLYEEALAQTG | 43.2 | 118 |
| 50 | AKPPGPYAEAQAQTG | 41.2 | 119 |
| 51 | AQAKGLYEAAQAQTG | 41.1 | 120 |
| 52 | AQNMTAAHYAQAQTG | 39.8 | 121 |
| 53 | AQAGGEYEEALAQTG | 39.2 | 122 |
| 54 | AQNDKMYWYGQAQTG | 39.1 | 123 |
| 55 | AQNDKMYEAAQAQTG | 38.5 | 124 |
| 56 | AQGKGLYEAAQAQTG | 38.1 | 125 |
| 57 | AQAKGDYEAAQAQTG | 37.9 | 126 |
| 58 | AQGGGDYDAALAQTG | 37.6 | 127 |
| 59 | AQAGGDYDAALAQTG | 37.1 | 128 |
| 60 | AQAKGDYEAVQAQTG | 37.0 | 129 |
| 61 | AQAKNPAQPALAQTG | 36.7 | 130 |
| 62 | AQEDKVYDAAQAQTG | 36.4 | 131 |
| 63 | AQGKGLYEAALAQTG | 35.8 | 132 |
| 64 | AQGKGDYEAAQAQTG | 35.7 | 133 |
| 65 | AQNGGMYAAAQAQTG | 29.8 | 134 |
| 66 | AQNGKGYAYAQAQTG | 29.8 | 135 |
| 67 | AQAGGEYEEVQAQTG | 29.7 | 136 |
| 68 | AQGGKGYAYAQAQTG | 29.4 | 137 |
| 69 | AKPPGPYAKAQAQTG | 29.3 | 138 |
| 70 | AKPPSPYAEAQAQTG | 27.6 | 139 |
| 71 | AQPPGPAAEAQAQTG | 27.1 | 140 |
| 72 | AQPPGPYAAAQAQTG | 26.2 | 141 |
| 73 | AQPPGPAAVAQAQTG | 25.9 | 142 |
| 74 | AKPPSPYAKAQAQTG | 24.8 | 143 |
| 75 | AQGKNPAQPAQAQTG | 24.2 | 144 |
| 76 | AQAGGDYEAAQAQTG | 23.0 | 145 |
| 77 | AQNGKMYYYAQAQTG | 22.5 | 146 |
| 78 | AQGGGDYDAVQAQTG | 22.4 | 147 |
| 79 | AQNKKMYAAAQAQTG | 22.0 | 148 |
| 80 | AQGKGGYEAAQAQTG | 19.5 | 149 |
| 81 | AKPPSPSAKAQAQTG | 18.1 | 150 |
| 82 | AQGGKGYEYAQAQTG | 17.8 | 151 |
| 83 | AKPPGPAAKAQAQTG | 17.7 | 152 |
| 84 | AKPPGPSAEAQAQTG | 17.6 | 153 |
| 85 | AKPPSPAAEAQAQTG | 16.9 | 154 |
| 86 | AQAGGDYDAAQAQTG | 16.6 | 155 |
| 87 | AKPPGPSAKAQAQTG | 16.2 | 156 |
| 88 | AQGKNPAQPALAQTG | 15.9 | 157 |
| 89 | AQGNNPAKPALAQTG | 15.3 | 158 |
| 90 | AQGGGDYEAAQAQTG | 13.7 | 159 |
| 91 | AQGKNPAQPVQAQTG | 12.6 | 160 |
| 92 | AKPPGPAAEAQAQTG | 12.5 | 161 |
| 93 | ATAPSGGGEGQAQTG | 12.0 | 162 |
| 94 | AKPPSPAAKAQAQTG | 11.8 | 163 |
| 95 | AQPKGPAAEAQAQTG | 10.1 | 164 |
| 96 | AQPPGPAAAAQAQTG | 8.8 | 165 |
| 97 | AQGNNPAKPVQAQTG | 8.5 | 166 |
| 98 | AKPPSPSAEAQAQTG | 8.2 | 167 |
| 99 | AQGGGDYDAAQAQTG | 6.9 | 168 |
| 100 | AQANNPAKPVQAQTG | 6.4 | 169 |
| 101 | AHADERADTAQAQTG | 5.5 | 170 |
| 102 | AQANNPAKPALAQTG | 4.9 | 171 |
| 103 | ASAPPKEDSSQAQTG | 4.1 | 172 |
| 104 | AQAKNPAQPAQAQTG | 3.1 | 173 |
| 105 | AQAKNPAQPVQAQTG | 2.9 | 174 |
| 106 | AQGNNPAKPAQAQTG | 0.0 | 175 |
| 107 | AQANNPAKPAQAQTG | 0.0 | 176 |
| 108 | AQEGAESDSAQAQTG | 0.0 | 177 |

Antibody and Peptide Derivatives

Disclosed herein also include antibodies or fragments thereof comprising an amino acid sequence having a binding specificity to a BBB crossing receptor disclosed herein (e.g., carbonic anhydrase IV or Ly6c1). Disclosed herein also includes a peptide derivative or a conjugate thereof, having specificity to a BBB crossing receptor disclosed herein (e.g., carbonic anhydrase IV or Ly6c1).

In some embodiments, the antibody or fragment thereof can further comprise an Fc domain. In some embodiments, the antibody or fragment thereof is a single-chain variable fragment (scFv), a single-domain antibody, an immunoglobulin molecule, a monoclonal antibody, a chimeric antibody, a CDR-grafted antibody, a humanized antibody, a Fab fragment, a Fab′ fragment, an F(ab′)2 fragment, an Fv fragment, a disulfide linked Fv, an scFv, a single domain antibody, a diabody, a multispecific antibody, a dual specific antibody, an anti-idiotypic antibody, a bispecific antibody, or a functionally active epitope-binding fragment thereof.

In some embodiments, the antibody or fragment thereof is a bispecific antibody comprising at least one Fab having specificity to the carbonic anhydrase IV. The bispecific antibody can comprise another binding site directed at a different antigen.

In some embodiments, an antibody or fragment thereof disclosed herein can comprise an amino acid sequence selected from SEQ ID NO: 11-12 and SEQ ID NOs: 70-174; or (2) an amino acid sequence having at least 80% sequence identity to an amino acid sequence selected from SEQ ID NOs: 11-12 and SEQ ID NOs: 70-174. In some embodiments, an antibody or fragment thereof disclosed herein can comprise an amino acid sequence selected from SEQ ID NO: 6-15 and 57; or (2) an amino acid sequence having at least 80% sequence identity to an amino acid sequence selected from SEQ ID NOs: 6-15 and 57.

In some embodiments, the antibodies or fragments thereof are not capable to elicit a deleterious immune response in a subject to be treated, e.g., in a human. In some embodiments, antibodies, fragments, variants, or derivatives thereof of the disclosure are modified to reduce their immunogenicity using techniques recognized in the art. For example, antibodies can be humanized, primatized, deimmunized, or chimeric antibodies.

In some embodiments, the antibodies, fragments, variants, or derivatives thereof can further comprise a chemical moiety not naturally associated with an antibody. For example, the antibody or fragment thereof can comprise a flexible linker or can be modified to add a functional moiety such as a detectable label. The antibodies, fragments, variants, or derivatives thereof can be modified, i.e., by the covalent or non-covalent attachment of a chemical moiety to the antibody such that the attachment does not interfere or prevent the antibody from binding to the epitope. In some embodiments, a chemical moiety can be conjugated to an antibody using any technique known in the art.

The present disclosure also provides isolated polynucleotides or nucleic acid molecules encoding the peptides, antibodies, fragments, variants or derivatives thereof of the disclosure. For example, the polynucleotides of the present disclosure can encode the heavy and light chain variable regions of the antibodies, fragments, variants or derivatives thereof on the same polynucleotide molecule or on separate polynucleotide molecules. In some embodiments, the polynucleotides of the present disclosure can encode portions of the heavy and light chain variable regions of the antibodies (e.g., the CDR regions), fragments, variants or derivatives thereof on the same polynucleotide molecule or on separate polynucleotide molecules.

### Payload Delivery Across the BBB

Disclosed herein include methods and delivery systems for delivering a payload (e.g., a therapeutic agent) to a nervous system. In some embodiments, the method comprises providing a targeting peptide capable of binding to a carbonic anhydrase IV or a derivative thereof. The targeting peptide can be part of a delivery system and the delivery system can comprise a payload to be delivered to a nervous system. The method can further comprise administering the delivery system to the subject.

In some embodiments, the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof. In some embodiments, the delivery system comprises a nanoparticle selected from lipid-based nanoparticles, polymeric nanoparticles, inorganic nanoparticles, surfactant-based emulsions, nanowires, silica nanoparticles, virus-like particles, peptide or protein-based particles, lipid-polymer particles, nanolipoprotein particles, and combinations thereof.

In some embodiments, the delivery system comprises a viral vector or a non-viral vector. For example, the viral vector can comprise an adenovirus vector, an adeno-associated virus (AAV) vector, a lentiviral vector, or a retrovirus vector. In some embodiments, the viral vector is an AAV vector and the target peptide can be part of a capsid protein of the AAV vector.

Adeno-Associated Virus (AAV) and Recombinant AAV (rAAV)

In some embodiments, the delivery system for delivering a payload across the BBB is an AAV vector. AAV is a replication-deficient parvovirus, the single-stranded DNA genome of which is about 4.7 kb in length including 145 nucleotide inverted terminal repeats (ITRs). The ITRs play a role in the integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for the production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

In some embodiments, the AAV vectors can comprise coding regions of one or more proteins of interest. The AAV vector can include a 5′ AAV ITR, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the AAV vector includes a posttranscriptional regulator-element downstream of the restriction site and upstream of the 3′ AAV ITR.

The viral vectors can include additional sequences that make the vectors suitable for replication and integration in eukaryotes. In other embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. In some embodiments, the AAV capsid protein can be derived from an AAV serotype selected from AAV9, AAV9 K449R (or K449R AAV9), AAV1, AAVrhlO, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3. l/hu.6, AAV3. l/hu.9, AAV3-9/rh.52, AAV3-1 l/rh.53, AAV4-8/r11.64, AAV4-9/rh. 54, AAV4-19/rh.55, AAV5-3/rh.57, AAV5-22/rh. 58, AAV7.3/hu.7, AAV16.8/hu.10, AAV16.12/hu.1 1, AAV29.3/bb.1, AAV29.5/bb.2, AAV106. l/hu.37, AAV1 14.3/hu.40, AAV127.2/hu.41, AAV127.5/hu.42, AAV128.3/hu.44, AAV130.4/hu.48, AAV145. l/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhErl.35, AAVhEr1.7, AAVhEr1.36, AAVhEr2.29, AAVhEr2.4, AAVhEr2. 16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

The AAV vector can be an AAV9 having an amino acid sequence of SEQ ID NO: 178 or an amino acid sequence having at least 70% sequence identity (e.g., at least 70%, 80%, 90%, 95%, 96%, 97%, 98%, 99% or higher) to an amino acid sequence of SEQ ID NO: 178.

In some embodiments, the AAV vectors disclosed herein can be used as AAV transfer vectors carrying a transgene encoding a protein of interest (e.g., the targeting peptide) for producing recombinant AAV viruses that can express the protein of interest in a host cell. Accordingly, disclosed herein also include recombinant AAV viruses (rAAV). The rAAV can comprise an AAV capsid protein described herein.

The rAAV can comprise a chimeric AAV capsid. A “chimeric” AAV capsid refers to a capsid that has an exogenous amino acid or amino acid sequence. The rAAV may comprise a mosaic AAV capsid. A “mosaic” AAV capsid refers to a capsid that made up of two or more capsid proteins or polypeptides, each derived from a different AAV serotype. The rAAV can be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype ban be pseudotyped, which means that the ITRs from a first AAV serotype (e.g., AAV1) are used in a capsid from a second AAV serotype (e.g., AAV9), wherein the first and second AAV serotypes are not the same. As a non-limiting example, a pseudotyped AAV serotype comprising the AAV1 ITRs and AAV9 capsid protein may be indicated AAV1/9. The rAAV may additionally, or alternatively, comprise a capsid that has been engineered to express an exogenous ligand binding moiety (e.g., receptor), or a native receptor that is modified.

In some embodiments, the rAAV capsid proteins comprise a substitution or insertion of one or more amino acids in an amino acid sequence of an AAV capsid protein. The rAAV capsid proteins described herein have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from AAV1, AAV2, rAAV3, AAV4, AAV5, AAV6, AAV7, AAV8, AAV9, AAV10, AAV11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

In some embodiments, the rAAV vectors disclosed herein can carry a transgene encoding a targeting peptide described herein that is capable of binding to a carbonic anhydrase IV or having a specificity to a carbonic anhydrase IV (e.g., Car4 or CA4). The targeting peptide can be part of a capsid of the rAAV. Disclosed herein also include AAV capsid proteins. The AAV capsid protein can comprise a targeting peptide disclosed herein.

The location of the targeting peptide within the capsid protein can vary. In some embodiments, the targeting peptide can be inserted between two adjacent amino acids in AA586-595 (e.g., between AA586 and AA587, AA587 and AA588, AA588 and AA589, AA589 and AA590, AA590 and AA591, AA591 and AA592, AA592 and AA593, AA593, AA594 and AA595) of AAV9 capsid protein or functional equivalents thereof in other AAV capsid proteins. The AAV vector can be a vector selected from AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8, rhesus isolate rh.10, or a variant thereof. In some embodiments, the AAV vector is AAV9 or a variant or a derivative thereof. For example, the AAV capsid protein comprises, or consists thereof, SEQ ID NO: 178 or an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 178.

The targeting peptide can be inserted between AA588-589 of AAV9 capsid protein or functional equivalents thereof in other AAV capsid proteins. The two adjacent amino acids can be AA588-589. In some embodiments, the targeting peptide is inserted between AA587-590 of AAV9 capsid protein or functional equivalents thereof in other AAV capsid proteins.

The targeting peptide can comprise (1) an amino acid sequence selected from SEQ ID NOs: 11-12 and SEQ ID NOs: 70-174; (2) an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from SEQ ID NOs: 11-12 and SEQ ID NOs: 70-174.

The targeting peptide can comprise (1) an amino acid sequence selected from SEQ ID NOs: 6-15 and 57; (2) an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from SEQ ID NOs: 6-15 and 57.

The binding specificity between the targeting peptide carried by the rAAV and the carbonic anhydrase IV can be screened using a multiplexed Cre-recombination-based AAV targeted evolution (CREATE) method (M-CREATE). In the M-CREATE, the rAAV nucleic acid contains a label sequence flanked by two lox sequences. The rAAV is administrated to an animal (e.g., mouse) with the gene encoding a Cre recombinase expressed only in a target cell (e.g., endothelial cells in brain). The label sequence of rAAV capable of entering the target cell is inverted by Cre recombinase expressed in the target cell. Because the nucleic acid of rAAV lacking specificity to the target cell has no access to Cre recombinase, its label sequence is not inverted. Thus, after sequencing the nucleic acid of rAAV recovered from the animal, the targeting peptide of rAAV containing an inverted label sequence has specificity to the target cell. M-CREATE method is described in detail in U.S. Patent Publication US20170166926A1, the content of which is incorporated herein by reference in its entirety for all purposes.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

Uses of AAV Vectors and rAAVs for Payload Delivery

Disclosed herein also includes compositions for use in the delivery of a payload (e.g., a pharmaceutical agent) to a target environment such as a nervous system of a subject. The composition can comprise an AAV comprising (1) an AAV capsid protein disclosed herein and (2) an agent to be delivered to a target environment (e.g., nervous system) of the subject.

The target environment can be the CNS, the peripheral nervous system (PNS), or a combination thereof. The target environment can be brain endothelial cells, neurons, capillaries in the brain, arterioles in the brain, arteries in the brain, or a combination thereof.

The agent to be delivered can comprise a nucleic acid, a peptide, a small molecule, an aptamer, or a combination thereof. The AAV vectors disclosed herein can be effectively transduced to a target environment (e.g., the CNS), for example, for delivering nucleic acids. In some embodiments, a method of delivering a nucleic acid sequence to the nervous system is provided. The protein can be part of a capsid of an AAV. The AAV can comprise a nucleic acid sequence to be delivered to a nervous system. One can then administer the AAV to the subject.

In some embodiments, the nucleic acid sequence to be delivered to a target environment (e.g., nervous system) comprises one or more sequences that would be of some use or benefit to the nervous system and/or the local of delivery or surrounding tissue or environment. In some embodiments, it can be a nucleic acid that encodes a protein of interest. The nucleic acid can comprise one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

In some embodiments, the vector can also comprise regulatory control elements known to one of skill in the art to influence the expression of the RNA and/or protein products encoded by the polynucleotide within desired cells of the subject.

Functionally, expression of the polynucleotide can be at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of the intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The heterologous nucleic acid can comprise a 5′ ITR and a 3′ ITR. The agent can comprise a DNA sequence encoding a protein (e.g., a trophic factor, a growth factor, or a soluble protein). The heterologous nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. In some embodiments, the silencing effector comprises one or more miRNA binding sites (e.g., miR-122 binding sites). miRNA binding sites are operably linked regulatory elements that are typically located in the 3′UTR of the transcribed sequence. Binding of miRNAs to the target transcript (in complex with the RNA-Induced Silencing Complex, RISC) can reduce the expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. In some embodiments, the gene of interest can be selected from SOD1, MAPT, APOE, HTT, C90RF72, TDP-43, APP, BACE, SNCA, ATXN1, ATXN2, ATXN3, ATXN7, SCN1A-SCN5A, and SCN8A-SCN11A. The heterologous nucleic acid further can comprise a polynucleotide encoding one or more secondary proteins, and the protein and the one or more secondary proteins can comprise a synthetic protein circuit. The heterologous nucleic acid can comprise a single-stranded AAV (ssAAV) vector or a self-complementary AAV (scAAV) vector.

The promoter can comprise a ubiquitous promoter. The ubiquitous promoter can be selected from a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, including but not limited to, a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be, or comprise, a muscle-specific promoter, e.g., an MCK promoter.

The promoter can comprise an intronic sequence. The promoter can comprise a bidirectional promoter and/or an enhancer. In some embodiments, the enhancer is be a CMV enhancer. One or more cells of a subject can comprise an endogenous version of a nucleic acid sequence (e.g., a gene), and the promoter can comprise or can be derived from the promoter of the endogenous version. In some embodiments, one or more cells of a subject comprise an endogenous version of the nucleic acid sequence, and the sequence is not truncated relative to the endogenous version.

The promoter can vary in length, for example be less than 1 kb. In other embodiments, the promoter is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 1 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

As used herein, a “protein of interest” can be any protein, including naturally-occurring and non-naturally occurring proteins. In some embodiments, a polynucleotide encoding one or more proteins of interest can be present in one of the AAV vectors disclosed herein, wherein the polynucleotide is operably linked with a promoter. In some instances, the promoter can drive the expression of the protein(s) of interest in a host cell (e.g., an endothelial cell). In some embodiments, the protein of interest is an anti-tau antibody, an anti-AB antibody, and/or ApoE isoform.

The protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The protein can comprise a disease-associated protein. In some embodiments, the level of expression of the disease-associated protein correlates with the occurrence and/or progression of the disease. The protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

In some embodiments, the nucleic acid can comprise a cDNA that encodes a protein to control or monitor the activity or state of a cell, and/or for assessing the state of a cell. The protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The protein can comprise a nuclear localization signal (NLS) or a nuclear export signal (NES).

The protein can comprise a CRE recombinase, GCaMP, a cell therapy component, a knock-down gene therapy component, a cell-surface exposed epitope, or any combination thereof. The protein can comprise a chimeric antigen receptor. The protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the nucleic acid can comprise a cDNA that encodes a protein for gene editing, or a guide RNA; or a DNA sequence for genome editing via homologous recombination. The protein can comprise a programmable nuclease. In some embodiments, the programmable nuclease is selected from: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The heterologous nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single-strand DNA or single-strand RNA. The targeting molecule can comprise a single guide RNA (sgRNA).

The rAAV disclosed herein can comprise one or more of the heterologous nucleic acids disclosed herein. The heterologous nucleic acid can comprise a polynucleotide encoding a protein. The nucleic acid can be or can encode an RNA agent. The heterologous nucleic acid can comprise a promoter operably linked to the polynucleotide encoding a protein. As disclosed herein, the gene is operatively linked with appropriate regulatory elements in some embodiments. The one or more genes of the heterologous nucleic acid can comprise an siRNA, an shRNA, an antisense RNA oligonucleotide, an antisense miRNA, a trans-splicing RNA, a guide RNA, a single-guide RNA, a crRNA, a tracrRNA, a trans-splicing RNA, a pre-mRNA, an mRNA, or any combination thereof. The one or more genes of the heterologous nucleic acid can comprise one or more synthetic protein circuit components. The one or more genes of the heterologous nucleic acid can comprise can entire synthetic protein circuit comprising one or more synthetic protein circuit components. The one or more genes of the heterologous nucleic acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof; erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; cystic fibrosis transmembrane conductance regulator (CFTR) and variants thereof; and interferons and variants thereof.

Examples of protein of interest include, but are not limited to, luciferases; fluorescent proteins (e.g., GFP); growth hormones (GHs) and variants thereof; insulin-like growth factors (IGFs) and variants thereof; granulocyte colony-stimulating factors (G-CSFs) and variants thereof erythropoietin (EPO) and variants thereof; insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof; CFTR and variants thereof; and interferons and variants thereof.

In some embodiments, the protein of interest is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (GF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-α receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Groa/IL-S, RANTES, M1P-1 a, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-rel easing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of nietalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor IX or Factor X; dystrophin or nini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

The protein of interest can be, for example, an active fragment of a protein, such as any of the aforementioned proteins, a fusion protein comprising some or all of two or more proteins, or a fusion protein comprising all or a portion of any of the aforementioned proteins.

In some embodiments, the viral vector comprises a polynucleotide comprising coding regions for two or more proteins of interest, The two or more proteins of interest can be the same or different from each other. In some embodiments, the two or more proteins of interest are related polypeptides, for example light chain(s) and heavy chain(s) of the same antibody.

The protein of interest can be a multi-subunit protein. For example, the protein of interest can comprise two or more subunits, or two or more independent polypeptide chains. In some embodiments, the protein of interest can be an antibody, including, but not limited to, antibodies of various isotypes (for example, IgG1, IgG2, IgG3, IgG, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody. In some embodiments, the protein of interest is not an immune-adhesin.

In some embodiments, the resulting targeting molecules can be employed in methods and/or therapies relating to in vivo gene transfer applications to long-lived cell populations. In some embodiments, these can be applied to any rAAV-based gene therapy, including, for example: spinal muscular atrophy (SMA), amyotrophic lateral sclerosis (ALS), Parkinson's disease, Friedreich's ataxia, Pompe disease, Huntington's disease, Alzheimer's disease, Battens disease, lysosomal storage disorders, glioblastoma multiforme, Rett syndrome, Leber's congenital amaurosis, chronic pain, stroke, spinal cord injury, traumatic brain injury and lysosomal storage disorders. In addition, rAAVs can also be employed for in vivo delivery of transgenes for non-therapeutic scientific studies such as optogenetics, gene overexpression, gene knock-down with shRNA or miRNAs, modulation of endogenous miRNAs using miRNA sponges or decoys, recombinase delivery for conditional gene deletion, conditional (recombinase-dependent) expression, or gene editing with CRISPRs, TALENs, and zinc finger nucleases.

In some embodiments, the gene encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments of the heterologous nucleic acids provided herein can be employed as vaccines for the prophylaxis or treatment of infectious diseases (e.g., as vaccines).

As described herein, the nucleotide sequence encoding the protein can be modified to improve the expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of gene expression in the target cell can vary. The amount of the protein expressed in the subject (e.g., the CNS of the subject) can vary. For example, in some embodiments the protein can be expressed in the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the protein is expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein is needed for the method to be effective can vary depending on non-limiting factors such as the particular protein and the subject receiving the treatment, and an effective amount of the protein can be readily determined by a skilled artisan using conventional methods known in the art without undue experimentation.

The agent can be an inducer of cell death. The agent can induce cell death by a non-endogenous cell death pathway (e.g., a bacterial pore-forming toxin). In some embodiments, the agent (e.g., a protein encoded by a nucleic acid) can be a pro-survival protein. In some embodiments, the agent is a modulator of the immune system. The agent can activate an adaptive immune response, and innate immune response, or both. In some embodiments, the nucleic acid encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments of the compositions provided herein can be employed as vaccines for the prophylaxis or treatment of infectious diseases (e.g., as vaccines). The protein can comprise a CRE recombinase, GCaMP, a cell therapy component, a knock-down gene therapy component, a cell-surface exposed epitope, or any combination thereof. In some embodiments, the protein comprises CFTR, GDE, OTOF, DYSF, MYO7A, ABCA4, F8, CEP290, CDH23, DMD, and ALMS1.

The agent can comprise a non-protein coding gene, such as an RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some embodiments, the RNA agent can comprise non-natural or modified nucleotides (e.g., pseudouridine). In some embodiments, the non-protein coding gene can modulate the expression or the activity of a target gene or gene expression product. For example, the RNAs described herein may be used to inhibit gene expression in a target cell, for example, a cell in the central nervous system (CNS). In some embodiments, inhibition of gene expression refers to inhibition by at least about 20%, 30%, 40%, 50%, 60%, 70%, 80%, 85%, 90%, 95% or 100%. In some cases, the protein product of the targeted gene is inhibited by at least about 20%, 30%, 40%, 50%, 60%, 70%, 80%, 85%, 90%, 95% or 99%. The gene can be either a wild type gene or a gene with at least one mutation. The targeted protein can be a wild type protein, or a protein with at least one mutation.

Examples of genes encoding therapeutic proteins include a sequence associated with a signaling biochemical pathway, e.g., a signaling biochemical pathway-associated gene or polynucleotide (e.g., a signal transducer). In some embodiments, the methods and compositions disclosed herein comprise knockdown of an endogenous signal transducer accompanied by tuned expression of a protein comprising an appropriate version of signal transducer. Examples of DNA or RNA sequences contemplated herein include sequences for a disease-associated gene or polynucleotide. A “disease-associated” gene or polynucleotide refers to any gene or polynucleotide which is yielding transcription or translation products at an abnormal level or in an abnormal form in cells derived from disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products can be known or unknown, and can be at a normal or abnormal level. Signal transducers can be associated with one or more diseases or disorders. In some embodiments, a disease or disorder is characterized by aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder.

Many proteins (e.g., enzymes) are secreted and can exert cross-correction effects. For these, the genetic material can be delivered to brain endothelial cells using an AAV of the present disclosure, transforming these cells into a biofactory to produce and distribute therapeutics to other cell types. For example, the production of the secreted Sparcl1/Hevin protein in brain endothelial cells can rescue the thalamocortical synapse loss phenotype of Hevin KO mice (See, Example 1 below). This proof-of-concept supports the brain endothelial cell biofactory model for the production of enzymes, antibodies, or other biological therapeutics, providing a novel therapeutic approach for diseases like lysosomal storage disorders.

In some embodiments, the rAAV having a capsid protein comprising one or more targeting peptides disclosed herein can be used to deliver genes to specific cell types in the target environment of a subject. For example, the rAAV can be used for delivering genes to neurons and glia in the nervous system (including PNS, CNS, or both) of a subject (e.g., a mammal). The compositions and methods disclosed herein can be used in, for example, (i) reducing the expression of mutant Huntingtin in patients with Huntington's Disease by, for example, incorporating a Huntingtin-specific microRNA expression cassette within a rAAV genome and packaging the rAAV genome into a variant rAAV for delivery through, for example the vasculature, (ii) delivering a functional copy of the Frataxin gene to patients with Friedreich's ataxia, (iii) restoring expression of an enzyme critical for normal lysosomal function in patients lacking expression of the enzyme due to genetic mutation (e.g., patients with Neimann-Pick disease, mucopolysaccharidosis III, and/or Gaucher's disease), (iv) using the rAAV to generate animal models of disease, or a combination thereof.

The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

In some embodiments, the subject is suffering from an acute condition or injury. The subject in need can be a subject suffering from, at risk to develop, or has suffered from a stroke, traumatic brain injury, epilepsy, or spinal cord injury.

### Pharmaceutical Compositions and Methods of Administration

Also disclosed herein are pharmaceutical compositions comprising one or more of the rAAV viruses disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Disclosed herein include methods of delivering an agent to a nervous system of a subject. In some embodiments, the method comprises: providing an AAV vector comprising an AAV capsid protein disclosed herein. In some embodiments, the AAV vector comprises an agent to be delivered to the nervous system. In some embodiments, the method comprises administering the AAV vector to the subject. The composition can be for intravenous administration. The composition can be for systemic administration. The agent can be delivered to endothelial lining of the ventricles in the brain, the central canal of the spinal cord, capillaries in the brain, arterioles in the brain, arteries in the brain, or a combination thereof of the subject. The subject can be an adult animal.

Titers of the rAAV to be administered will vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration will vary depending upon the age, weight, severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact dosage can be determined on a drug-by-drug basis, in most cases, some generalizations regarding the dosage can be made. In some embodiments, the rAAV for delivery of an agent to the nervous system (e.g., CNS) of a subject can be administered, for example via injection, to a subject at a dose of between 1×10¹⁰ viral genome (vg) of the recombinant virus per kg of the subject and 2×10¹⁴ vg per kg, for example between 5×10¹¹ vg/kg and 5×10¹² vg/kg. In some embodiments, the dose of the rAAV administered to the subject is no more than 2×10¹⁴ vg per kg. In some embodiments, the dose of the rAAV administered to the subject is no more than 5×10¹² vg per kg. In some embodiments, the dose of the rAAV administered to the subject is no more than 5×10¹¹ vg per kg.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some embodiments, pharmaceutical compositions in accordance with the present disclosure are administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations can be converted to vg or viral genomes per kg or into total viral genomes administered by one of skill in the art.

In some embodiments, a dose of the pharmaceutical composition comprises a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹¹, 9×10¹³, 9×10¹⁴, 9×10¹-5, 9×10¹⁶, 9×10¹⁷, or a range between any two of these values.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The administration can be a systemic administration. The administration can be an intravenous administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. In some embodiments, the recombinant virus is administered to the subject by systematic transduction. In some embodiments, the recombinant virus is administered to the subject by intramuscular injection. In some embodiments, the rAAV is administered to the subject by the parenteral route (e.g., by intravenous, intramuscular or subcutaneous injection), by surface scarification or by inoculation into a body cavity of the subject. Route(s) of administration and serotype(s) of AAV components of the rAAV virus can be readily determined by one skilled in the art taking into account the infection and/or disease state being treated and the target cells/tissue(s) that are to express the protein of interest. In some embodiments, it can be advantageous to administer the rAAV via intravenous administration. The variant AAV provided herein can advantageously provide for intravenous administration of vectors with enhanced tropisms for CNS.

In some embodiments, the subject is a primate and the agent is delivered to the endothelial cells and/or neurons of the nervous system. The nervous system can be the central nervous system (CNS). The agent can be delivered to the endothelial cells of the nervous system of the subject at least 1.5-fold, 2-fold, or 3-fold more efficiently than the delivery of the agent to the neurons of the nervous system. In some embodiments, the agent is delivered to the endothelial cells of the nervous system of the subject more than 3-fold more efficiently (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the neurons of the nervous system.

Disclosed herein include methods of delivering an agent to a cell. In some embodiments, the method comprises: contacting an AAV vector comprising an AAV capsid protein disclosed herein with the cell. In some embodiments, the AAV vector comprises an agent to be delivered to the nervous system. In some embodiments, the cell is an endothelial cell or a neuron. In some embodiments, contacting the AAV vector with the cell occurs in vitro, in vivo or ex vivo. The cell can be present in a tissue, an organ, or a subject. The cell can be a brain endothelial cell, a neuron, a cell in the capillaries in the brain, a cell in the arterioles in the brain, a cell in the arteries in the brain, a cell in the brain vasculature, or a combination thereof.

The AAV vector can be an AAV9 vector, or a variant thereof. In some embodiments, the AAV vector is a vector selected from AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

The variant AAV capsid can comprise tropism for a tissue or a cell of a central nervous system (CNS). The target cell can be a neuronal cell, a neural stem cell, an astrocytes, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell.

Actual administration of the rAAV can be accomplished by using any physical method that will transport the rAAV into the nervous system of the subject. For example, the rAAV disclosed herein can advantageously be administered intravenously for delivery to the CNS. As disclosed herein, capsid proteins of the rAAV can be modified so that the rAAV is targeted to a particular target environment of interest such as central nervous system, and to enhance tropism to the target environment of interest (e.g., CNS tropism). Pharmaceutical compositions can be prepared, for example, as injectable formulations.

The recombinant virus to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the rAAV have been established for at least some condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the rAAV can be administered to a subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with CNS, which includes but is not limited to Krabbe disease, Sandhoff disease, Tay-Sachs, Gaucher disease (Type I, II, or 111), Niemann-Pick disease (NPC1 or NPC2 deficiency), Hurler syndrome, Pompe disease, Batten disease, or any combination thereof), chronic pain, or a combination thereof. In some embodiments, the rAAV is administered to the subject during remission of the disease or disorder. In some embodiments, the rAAV is administered prior to the onset of the disease or disorder in the subject. In some embodiments, the rAAV is administered to a subject at a risk of developing the disease or disorder.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, or any combination thereof.

Disclosed herein, in some embodiments, are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some instances, the rAAV compositions are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. In some embodiments, the rAAV disclosed herein can advantageously be administered intravenously for delivery to the CNS.

In some embodiments, the pharmaceutical forms of the AAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Formulations for intranasal administration can comprise a coarse powder comprising the active ingredient and having an average particle size from about 0.2 μm to 500 μm. Such formulations are administered in the manner in which snuff is taken, e.g. by rapid inhalation through the nasal passage from a container of the powder held close to the nose. Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, comprise 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions are within the purview of the skilled artisan having benefit of the present teachings. In some embodiments, however, that the administration of therapeutically-effective amounts of the disclosed compositions may be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In some embodiments, it is advantageous to provide multiple, or successive administrations of the AAV vector compositions, either over a relatively short, or a relatively prolonged period of time, as may be determined by the medical practitioner overseeing the administration of such compositions. For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in certain embodiments, it may be desirable to administer two or more different AAV vector compositions, either alone, or in combination with one or more other therapeutic drugs to achieve the desired effects of a particular therapy regimen. In various embodiments, the daily and unit dosages are altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The targeting peptides described herein can be used to generate rAAVs with enhanced CNS tropisms with capsid proteins derived from different AAV serotypes (e.g., AAV9 and AAV1). In some embodiments, this can advantageously provide for administration of two or more different AAV vector compositions without inducing immune response in the subject.

The dosing frequency of the rAAV virus can vary. For example, the rAAV virus can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the rAAV virus is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

Disclosed herein are kits comprising compositions disclosed herein. Also disclosed herein are kits for the treatment or prevention of a disease or conditions of the CNS, PNS, or target organ or environment (e.g., CNS). In some instances, the disease or condition is cancer, a pathogen infection, neurological disease, muscular disease, or an immune disorder, such as those described herein. In one embodiment, a kit can include a therapeutic or prophylactic composition containing an effective amount of a composition of a rAAV particle encapsidating a heterologous nucleic acid provided herein and a rAAV capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, rAAV are provided together with instructions for administering the rAAV to a subject having or at risk of developing the disease or condition. Instructions can generally include information about the use of the composition for the treatment or prevention of the disease or condition.

The kit can include allogenic cells. In some embodiments, a kit includes cells that can comprise a genomic modification. In some embodiments, a kit comprises “off-the-shelf” cells. In some embodiments, a kit includes cells that can be expanded for clinical use. In some embodiments, a kit contains contents for a research purpose.

In some embodiments, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some embodiments, instructions provide procedures for administering the rAAV to the subject alone. In some embodiments, instructions provide procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. In some instances, the instructions provide that the rAAV is formulated for intravenous injection. In some instances, the instructions provide that the rAAV is formulated for intranasal administration.

### EXAMPLES

Some aspects of the embodiments discussed above are disclosed in further detail in the following examples, which are not in any way intended to limit the scope of the present disclosure.

### Example 1

### Materials and methods

The following experimental materials and methods were used for Examples 2 and 3 described below.

### Plasmids

AAV capsid variants were subcloned into the pUCmini-iCAP-PHP.B backbone (Addgene ID: 103002). ssAAV genomes employed were pAAV:CAG-mNeonGreen (Addgene ID: 99134) and pAAV:CAG-2×NLS-EGFP (equivalent version with one NLS: Addgene ID: 104061), as noted in figures and legends. Receptor candidate plasmids were purchased from GenScript. All selected open reading frame (ORF) clones were introduced to a pcDNA3.1+/C-(K)-DYK backbone, except Lynx1 in pcDNA3.1. AAV capsid libraries were amplified from pCRII-9Cap-XE plasmid and subcloned into rAAV-DCap-in-cis-Lox2 plasmid for transfection with AAV2/9 REP-AAP-DCap.

### Animals

All animal procedures were approved by the California Institute of Technology Institutional Animal Care and Use Committee (IACUC) and complied with all relevant ethical regulations. C57BL/6J (000664), BALB/cJ (000651), 12951/SvlmJ (002448), CBA/J (000656), DBA/2J (000671), NOD/ShiLtJ (001976), Syn1-Cre (3966), GFAP-Cre (012886), Tek-Cre (8863), and Olig2-Cre (025567) mouse lines were purchased from Jackson Laboratory (JAX). Heterozygous Car4 knockout mice (008217) were cryo-recovered by JAX and bred at Caltech to generate homozygous WT/WT and KO/KO animals. Six to eight week old male mice were intravenously injected with rAAV into the retro-orbital sinus. Mice were randomly assigned with a particular rAAV during the testing of transduction phenotypes. Experimenters were not blinded for any of the experiments performed.

### AAV Vector Production

AAV packaging and purification were performed as previously described. Briefly, rAAVs were produced by triple transfection of HEK293T cells (ATTC, CRL-3216) using polyethylenimine. Media was collected at 72 h and 120 h post-transfection. Viruses were precipitated in 40% polyethylene glycol in 2.5 M NaCl. The viruses were resuspended and combined with 120 h post-transfection cell pellets at 37° C. in 500 mM NaCl, 40 mM Tris, 10 mM MgCl₂, and 100 U mL⁻¹ salt-active nuclease (ArcticZymes, 70910-202). The resulting lysate was extracted from an iodixanol (Cosmo Bio USA, OptiPrep, AXS-1114542) step gradient following ultracentrifugation. The purified viruses were concentrated and buffer exchanged with phosphate buffered saline (PBS) prior to titer determination by quantitative PCR.

### AAV Vector Administration, Tissue Processing, and Imaging

AAV vectors were administered intravenously to adult male mice via retro-orbital injection at doses of 1×10¹¹ or 3×10¹¹ v.g. as indicated in figures and legends. After three weeks of expression, mice were anesthetized with Euthasol (pentobarbital sodium and phenytoin sodium solution, Virbac AH) and transcardially perfused with roughly 50 mL of 0.1 M PBS, pH 7.4, and then another 50 mL of 4% paraformaldehyde (PFA) in 0.1 M PBS. Organs were then harvested and post-fixed in 4% PFA overnight at 4° C. before being washed and stored in 0.1 M PBS and 0.05% sodium azide at 4° C. Finally, the brains were cut into 100 μm sections on a Leica VT1200 vibratome. Images were acquired with a Zeiss LSM 880 confocal microscope using a Plan-Apochromat×10 0.45 M27 (working distance, 2.0 mm) objective and processed in Zen Black 2.3 SP1 (Zeiss) and ImageJ software.

### Single-Cell RNA Sequencing Analysis

Analysis was performed on a pre-existing C57BL/6J cortex single-cell RNA sequencing dataset with custom-written scripts in Python 3.7.4 using a custom fork off of scVI v0.8.1, and Scanpy v1.6.0 as described previously. Briefly, droplets that passed quality control were classified as ‘neurons’ or ‘non-neurons’ using a trained scANVI cell type classifier, retaining only those cells above a false discovery rate threshold of 0.05 after correction for multiple comparisons. Non-neuronal cells were further subtyped using a trained scVI model and clustered based on the learned latent space using the Leiden algorithm as implemented in Scanpy. Endothelial cell clusters were assigned if they were positive for all marker genes for that cell subtype. Membrane proteins were filtered by Uniprot keyword ‘cell membrane.’ Differential expression scores were calculated in Scanpy.

### Immunofluorescence

Immunofluorescence experiments were performed on HEK293T cells to label transiently transfected receptors such as Ly6a (Abcam ab51317, 1:200 dilution), Ly6c1 (Abcam ab15627, 1:200 dilution), and Car4 (Invitrogen PA5-47312, 1:40 dilution). HEK293T cells were seeded at 80% confluency in 6-well plates and maintained in Dulbecco's Modified Eagle Medium (DMEM) supplemented with 5% fetal bovine serum (FBS), 1% non-essential amino acids (NEAA), and 100 U/mL penicillin-streptomycin at 37° C. in 5% CO₂. Membrane-associated receptor candidates were transfected by polyethylenimine (PolySciences #23966). Cells were seeded on Neuvitro Poly-D-lysine coated sterile German glass coverslips (Fisher Scientific #NC0343705) 24 hours post-transfection in 24-well plates and then fixed in 4% paraformaldehyde once attached. Coverslips were blocked with 1× tris-buffered saline (TBS) containing 3% bovine serum albumin (BSA) for 30 minutes and incubated in primary antibody in 1×TBS, 3% BSA, and 0.05% Triton X-100 for 60 minutes at ambient temperature. Coverslips were washed three times in 1×TBS and then incubated with secondary antibodies (Ly6a & Ly6c1: Invitrogen A-21247, 1:1000 dilution; Car4: Invitrogen A-21432, 1:1000 dilution) in the same medium for 60 minutes. Coverslips were mounted on slides with Diamond Antifade Mounting Media with DAPI (Invitrogen P36931). Fluorescent microscopic images were captured on a confocal laser-scanning microscope (LSM 880, Carl Zeiss, USA).

Cell Culture Characterization of rAAV Vectors

HEK293T cells were seeded at 80% confluency in 6-well plates and maintained in DMEM supplemented with 5% FBS, 1% NEAA, and 100 U mL-1 penicillin-streptomycin at 37° C. in 5% CO₂. Membrane-associated receptor candidates were transiently expressed in HEK293T cells by transfecting each well with 2.53 μg plasmid DNA. Receptor-expressing cells were transferred to 96-well plates at 20% confluency and maintained in FluoroBrite™ DMEM supplemented with 0.5% FBS, 1% NEAA, 100 U mL⁻¹ penicillin-streptomycin, 1× GlutaMAX, and 15 μM HEPES at 37° C. in 5% CO₂. Cells expressing each receptor candidate were transduced with engineered AAV variants at 1E9 v.g. well⁻¹ and 5E8 v.g. well⁻¹ in triplicate. Plates were imaged 24 hours post-transduction with the Keyence BZ-X700 using the 4×objective and NucBlue™ Live ReadyProbes™ Reagent (Hoechst 33342) to autofocus each well.

### Cell Culture Fluorescence Image Quantitation

All image processing was performed using a custom Python image processing pipeline. In brief, the areas of cells were determined in both brightfield and signal images. The percentage of cells transduced and brightness per transduced area were determined from these images.

First, background subtraction was performed on the brightfield images by applying gaussian blur (skimage.filters.gaussian, sigma=30, truncate=0.35) and subtracting the product from the original brightfield images. In brightfield images, cells were silhouetted by the lamp producing both bright and dark edges. Histogram-based thresholding was applied to these images to determine bright and dark regions of the brightfield images, which can be combined to create a mask of cell edges in the images. Cells can be filled by applying skimage.morphology.closing, which ran a template over the images to fill contiguous regions (skimage.morphology.disk, radius=2). Then, the total area of cells in the brightfield images can then determined by summing all the pixels in the mask.

On the signal images, background subtraction was performed by applying gaussian blur (skimage.filters.gaussian, sigma=100). Subtracting the product of gaussian blur from the original signal images produced images with minimal fluctuations in background intensity. Histogram-based thresholding was applied to these images to identify the intensity of the background in the brightfield images and created a mask of bright regions in the images, which comprised transduced cells. The noise was removed from the mask using skimage.morphology.remove_small_objects (min_size=5). From this, the total area of transduced cells was determined by summing all the pixels in the mask.

After performing this segmentation, the percentage of cells transduced was determined by taking the ratio of signal area to the total cell area. By multiplying the mask by the original image and summing all the pixel intensities in the product image, the total brightness of transduced cells was determined. This value was then divided by the total area of transduced cells to determine the brightness per transduced area.

### Receptor Protein Production

Ly6a-Fc was produced in Expi293F suspension cells grown in Expi293 Expression Medium (Thermo Fisher Scientific) in a 37° C., 5% CO₂ incubator with 130 rpm shaking. Transfection was performed with Expifectamine according to the manufacturer's instructions (Thermo Fisher Scientific). Following harvesting of cell-conditioned media, 1 M Tris, pH 8.0 was added to a final concentration of 20 mM. Ni-NTA Agarose (QIAGEN) was added to ˜5% conditioned media volume. 1 M sterile PBS, pH 7.2 (GIBCO) was added to ˜3× conditioned media volume. The mixture was stirred overnight at 4° C. Ni-NTA agarose beads were collected in a Buchner funnel and washed with ˜300 mL protein wash buffer (30 mM HEPES, pH 7.2, 150 mM NaCl, 20 mM imidazole). Beads were transferred to an Econo-Pak Chromatography column (Bio-Rad) and proteins were eluted in 15 mL of elution buffer (30 mM HEPES, pH 7.2, 150 mM NaCl, 200 mM imidazole). Proteins were concentrated using Amicon Ultracel 10K filters (Millipore). Absorbance at 280 nm was measured using a Nanodrop 2000 spectrophotometer (Thermo Fisher Scientific) to determine protein concentration.

### Surface Plasmon Resonance (SPR)

SPR was performed using a Sierra SPR-32 (Bruker). Ly6a-Fc fusion protein in HBS-P+ buffer (GE Healthcare) was immobilized to a protein A sensor chip at a capture level of approximately 1200-1500 response units (RUs). Two-fold dilutions of rAAVs beginning at 2×10¹² v.g. mL⁻¹ were injected at a flow rate of 10 μl min⁻¹ with a contact time of 240 s and a dissociation time of 600 s. After each cycle, the protein A sensor chip was regenerated with 10 mM glycine pH 1.5. Kinetic data were double reference subtracted.

### Automated Pairwise Peptide Receptor Analysis for Screening Engineered AAVs (APPRAISE-AAV)

FASTA-format files containing a target receptor amino acid sequence (mature protein part only) as well as peptide sequences corresponding to amino acids 587 through 594 (wild-type AAV9 VP1 indices) from two AAV capsids of interest were used for structural prediction using a batch version of ColabFold (alphafold-colabfold 2.1.14), a cloud-based implementation of multiple sequence alignment, and AlphaFold2 Multimer. The ColabFold Jupyter notebook was run on a Google Colaboratory session using GPU (NVIDIA Tesla V100 SXM2 16 GB; it was found that the same model of the GPU yielded the most consistent results). Alphafold2-multimer-v2 was chosen as the default AlphaFold version unless otherwise specified. Each model was recycled three times, and ten models were generated from each competition. Models were quantified with PyMol (version 2.3.3) using a custom script to count the total number of atoms in the interface (N_{contact}^{POI}, defined by a distance cutoff of 5 angstroms), the total number of atoms in the peptide that were clashing with the receptor (N_{clash}^{POI}, defined by a distance cutoff of 1 Å), the binding angle of the peptide (θ, defined as the angle between the vector from receptor gravity center to receptor anchor and the vector from receptor gravity center to peptide gravity center), and the binding depth (d, defined as the difference of the distance between the closest point on the peptide to the receptor center and the minor radius of the ellipsoid hull of the receptor normalized by the minor radius) of the peptide in each putative peptide-receptor complex model. The minor radii of the ellipsoid hulls of receptors were measured using HullRad 8.1 (Ly6a: 13.4 Å, Ly6c1: 12.7 Å, mouse Car4: 23.0 Å). Finally, the metric ΔB^{POI, competitor} for ranking the propensity of receptor binding was calculated by subtracting the total binding score of the peptide of interest by the counterpart score of the competing peptide:

ΔB^{POI,competitor}=B^{POI}−B^{competitor}=max(B_{energetic}^{POI}+B_{angle}^{POI}+B_{depth}^{POI},0)−max(B_{energetic}^{competitor}+B_{angle}^{competitor}+B_{depth}^{competitor},0), (5)

where the individual terms are defined as follows:

$${B}_{energetic}^{peptide} = max ( {N}_{contact}^{peptide} - {10}^{3} \cdot {N}_{clash}^{peptide} , 0 ) \qquad ( 2 )$$

$${B}_{angle}^{peptide} = \{ \begin{matrix}- {10}^{3} \cdot {( 1 - \frac{θ}{\frac{π}{2}} )}^{10} , if θ < \frac{π}{2} \\ 0 , if \frac{π}{2} <= θ <= π\end{matrix} \qquad ( 3 )$$

$${B}_{depth}^{peptide} = {10}^{2} \cdot {d}^{3} \qquad ( 4 )$$

The mean number of this metric across replicates was used to form a matrix and plot a heatmap. Peptides in the heatmap were ranked by the total number of competitions each peptide won minus the total number of competitions it lost (competitions with ΔB^{POI, competitor} scores that have p-values greater than 0.05 in the one-sample Student's t-test were excluded).

### Computational Structure Modeling of Receptor-AAV Complexes

Peptide-receptor structures were modeled using a similar procedure as described in the APPRAISE-AAV section but with only one single peptide of interest in the input file to achieve higher accuracy.

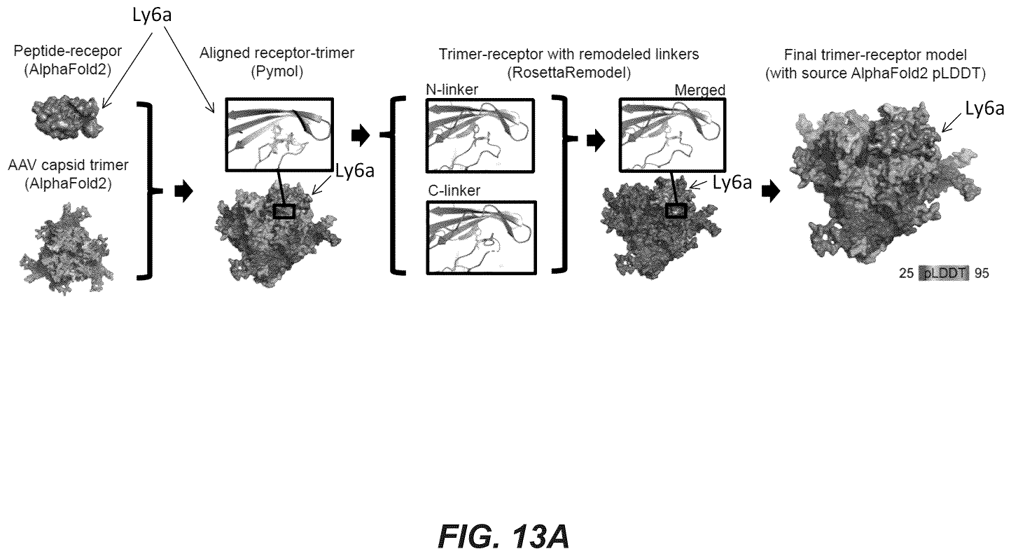

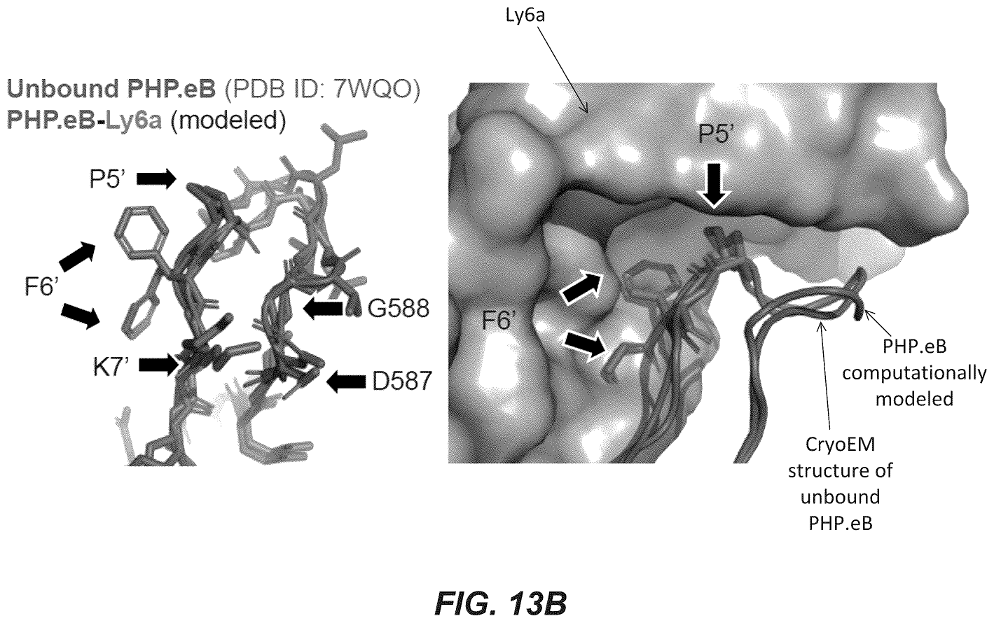

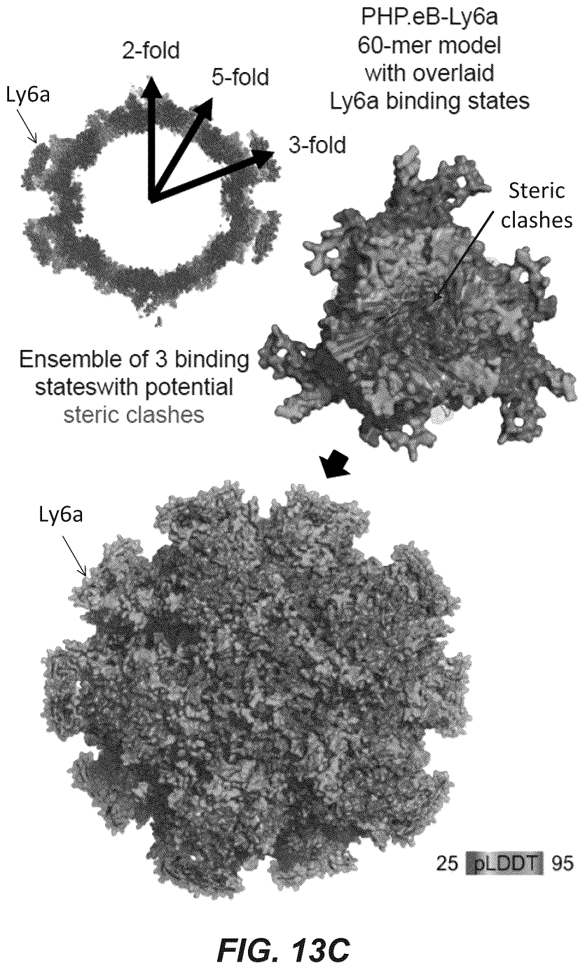

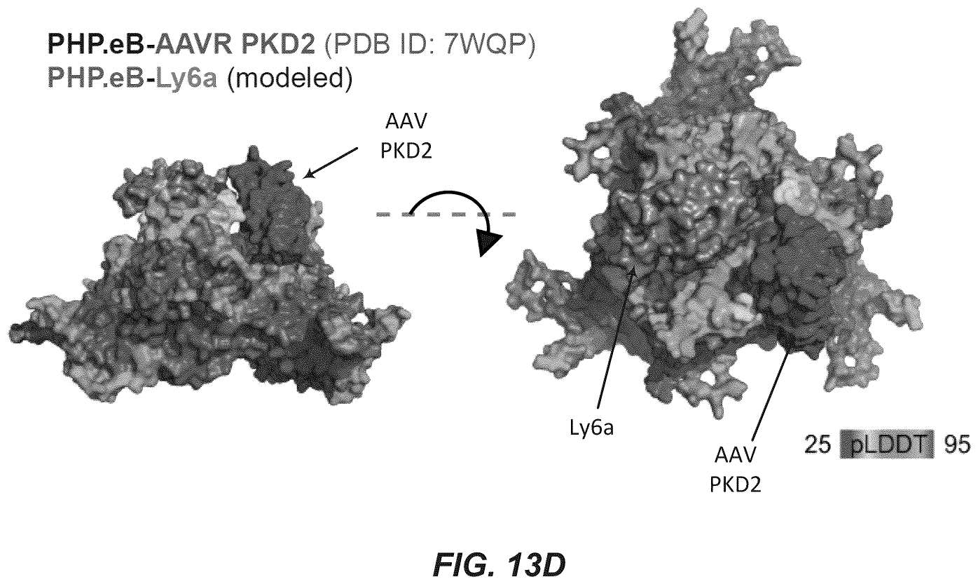

AAV trimer-receptor complex models were produced using an integrative structure modeling method (FIG. 13A). Trimers at the AAV three-fold symmetry interface were chosen as the minimal complete binding interface with a putative receptor that might recapitulate the entire viral particle while optimizing computational efficiency. First, a peptide-receptor model was generated by modeling the 15mer peptide sequence between the residues 587 and 594 (both in wild-type AAV9 VP1 indices) from the AAV variant of interest in complex with the target receptor as described above. Then, a trimer model of the AAV variant of interest was modeled using AlphaFold2 Multimer. The two residues with the highest confidence score (pLDDT score) in the 15mer peptide of the peptide-receptor model, Pro5′ and Phe6′, were structurally aligned to the corresponding residues on the first chain of the trimer model. A coarse combined model was then generated by combining the receptor and the two high-confidence AAV residues from the peptide receptor model with the remaining AAV residues from the trimer model. The two loops between Pro5′ and Phe6′ and the high-confidence AAV9 backbone in the coarse combined model (corresponding to residues 588-(588+)4′ and residues (588+)7′-590, respectively) were then individually remodeled using RosettaRemodel from the Rosetta software bundle (release 2018.48.60516). Finally, these remodeled loops were merged to generate a final model. The pLDDT scores for each residue from the original AlphaFold2 outputs were used to color images of the final model.

### M-CREATE Selections for PHP.eC

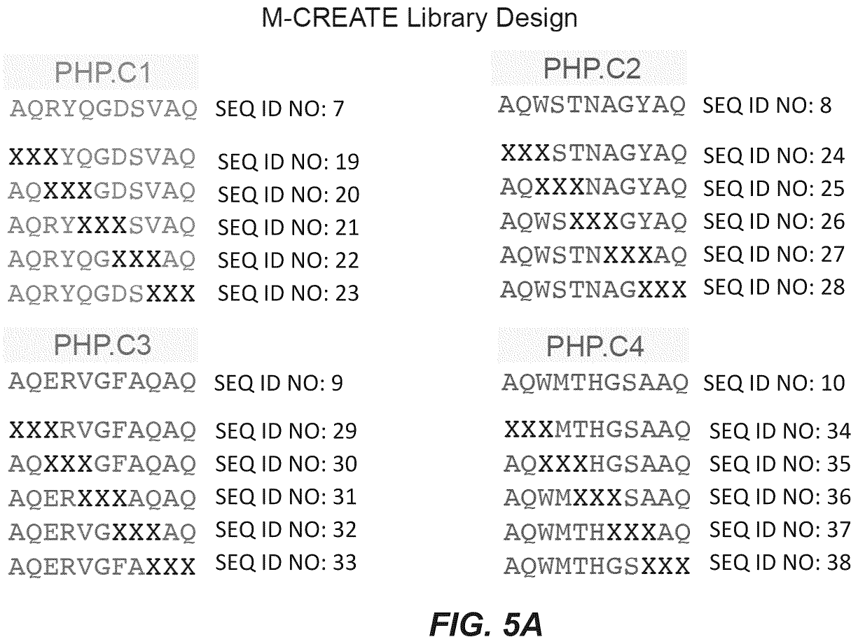

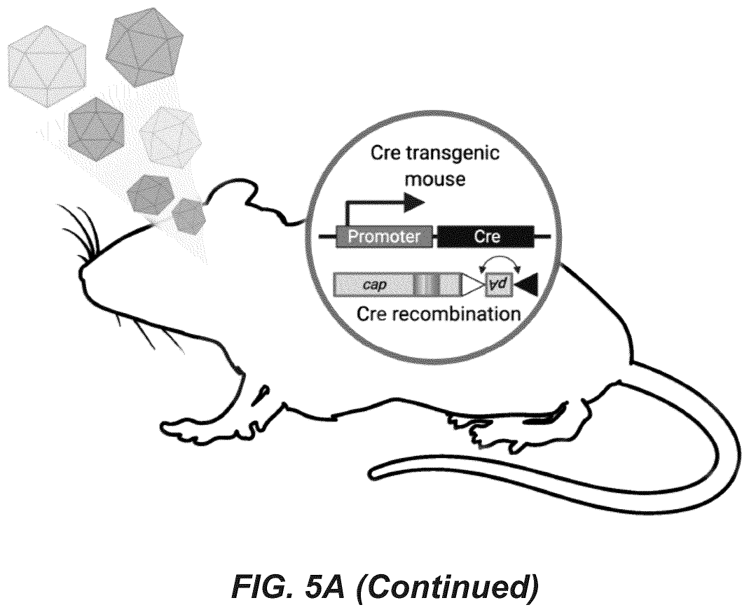

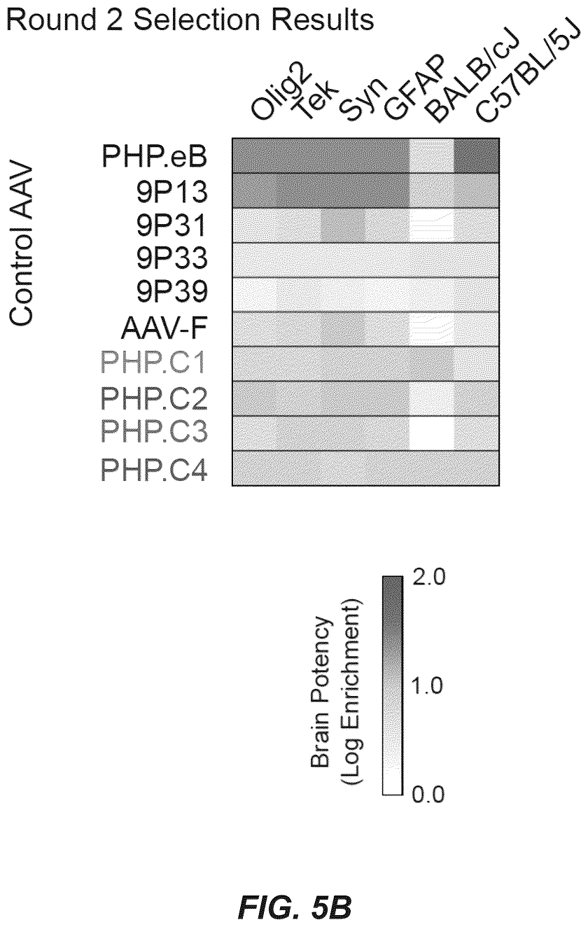

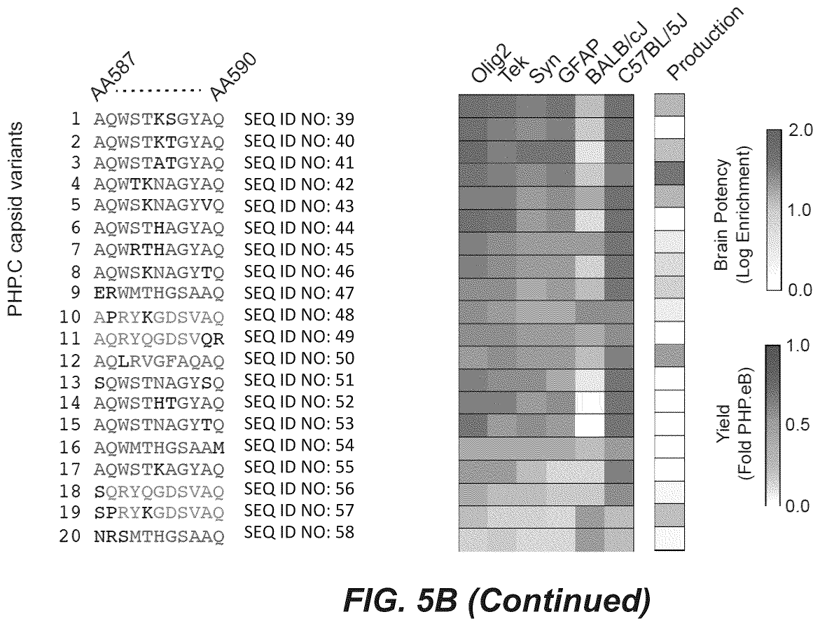

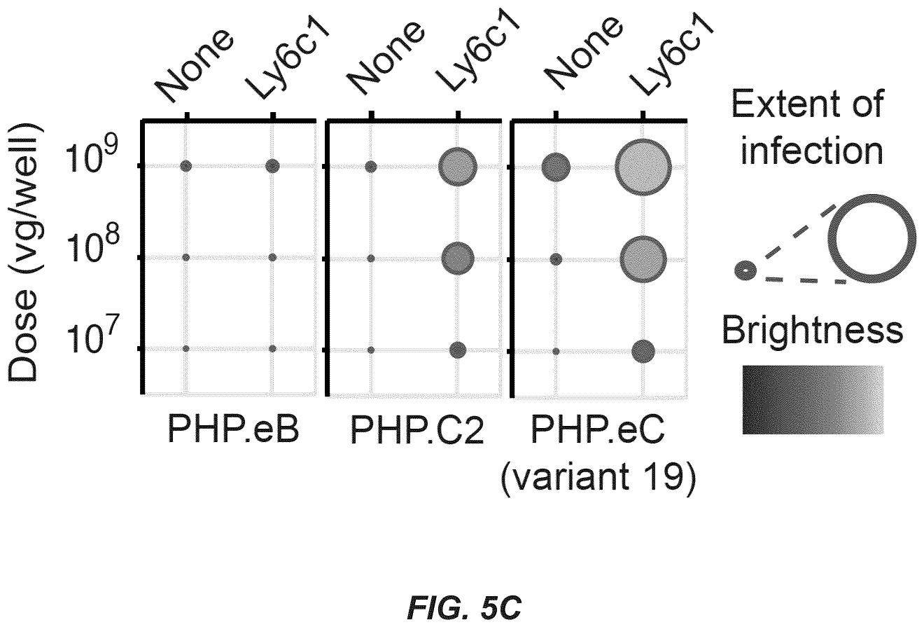

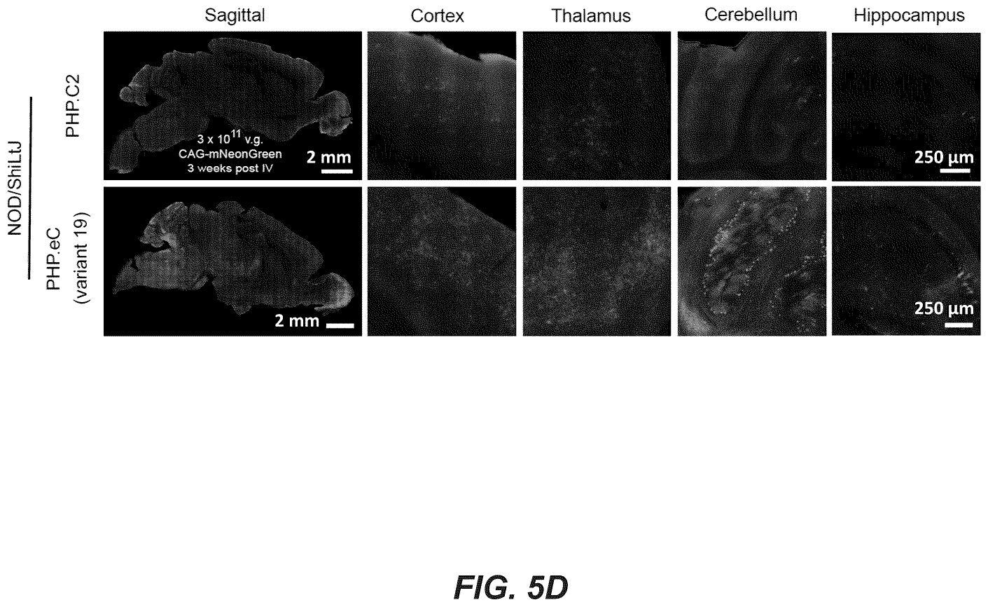

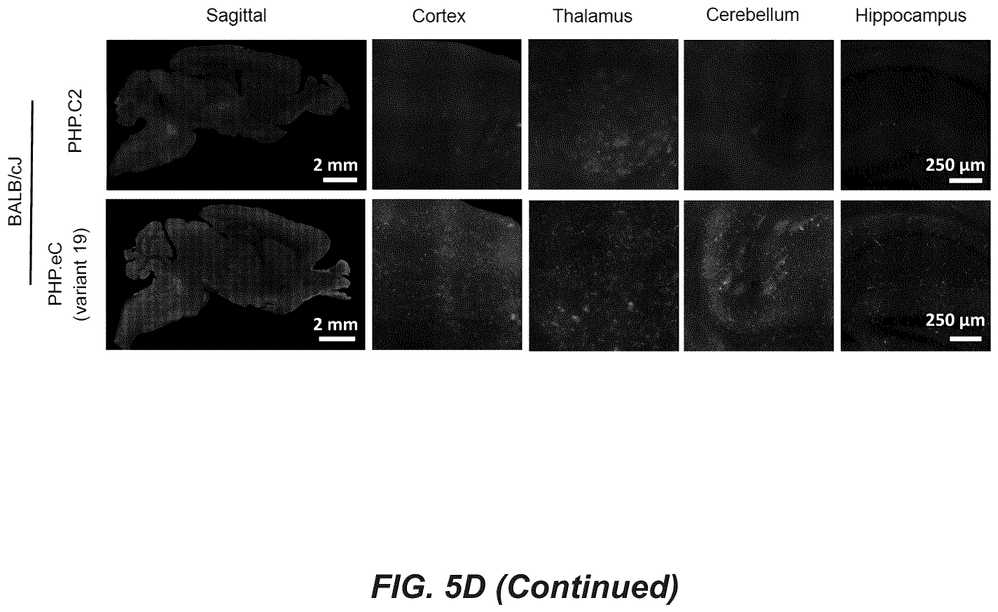

AAV capsid libraries were produced, administered, and recovered post-selection in vivo for next-generation sequencing as described previously. Briefly, an initial library was generated by pooling three amino acid NNK substitutions that scan from AAV9 587 through the 7mer insertion to AAV9 position 590 of AAV-PHP.C1, C2, C3, and C4 (FIG. 5A). A custom-designed synthetic round 2 library containing degenerate codon duplicates of 5515 capsid variants identified from the round 1 library post-selection and spike-in controls was synthesized in an equimolar ratio by Twist Biosciences. To prevent capsid mosaicism, only 10 ng of the assembled library was transfected per 150 mm dish of 293T producer cells and assembled capsids were purified at 60 h post-transfection. Round 1 library was retro-orbitally injected at 5×10¹⁰ v.g. per mouse in Syn-Cre mice, while the round 2 library was retro-orbitally injected at 5×10¹¹ v.g. per mouse in Syn-Cre, GFAP-Cre, Tek-Cre, Olig2-Cre, wild-type C57BL/6J, and wild-type BALB/cJ mice.

Two weeks post-injection, mice were euthanized and all organs including brains were collected, snap-frozen on dry ice, and stored at −80° C. Frozen tissues were homogenized using Beadbug homogenizers (Homogenizers, Benchmark Scientific, D1032-15, D1032-30, D1033-28) and rAAV genomes were Trizol extracted. Purified rAAV DNA (Zymo DNA Clean and Concentrator Kit D4033) was amplified by PCR using Cre-dependent primers, adding flow cell adaptors around the diversified region for next-generation sequencing. NGS data were aligned and processed as described previously to extract round 1 variant sequence counts and round 2 variant enrichment scores.

### Example 2

### Identification of Carbonic Anhydrase IV and Murine-Restricted Ly6c1 as New Targets for Crossing the Blood-Brain Barrier

This example demonstrates the identification of carbonic anhydrase IV and murine-restricted Ly6c1 as two novel targets for crossing the blood-brain barrier. By screening a curated pool of 40 candidate receptors selected for the intersection of their CNS expression level and endothelial-cell specificity, Ly6c1 and carbonic anhydrase IV were identified as molecular receptors for enhanced BBB crossing of ten Ly6a-independent engineered AAVs (as well as Ly6a-dependent PHP.N). These findings allow for more efficient allocation of NHPs, inform future directed evolution library designs, and enable receptor-guided engineering directly for human protein interactions.

Identification of Engineered AAVs that do not Utilize Ly6a

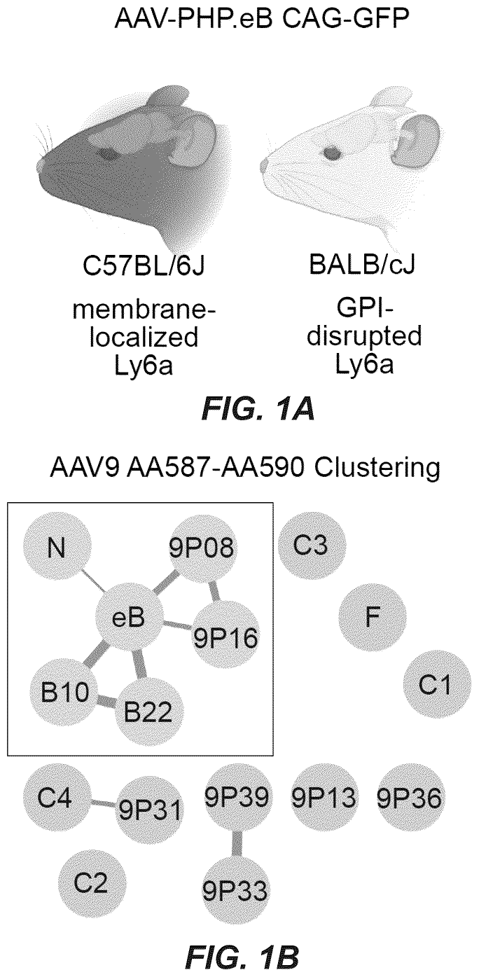

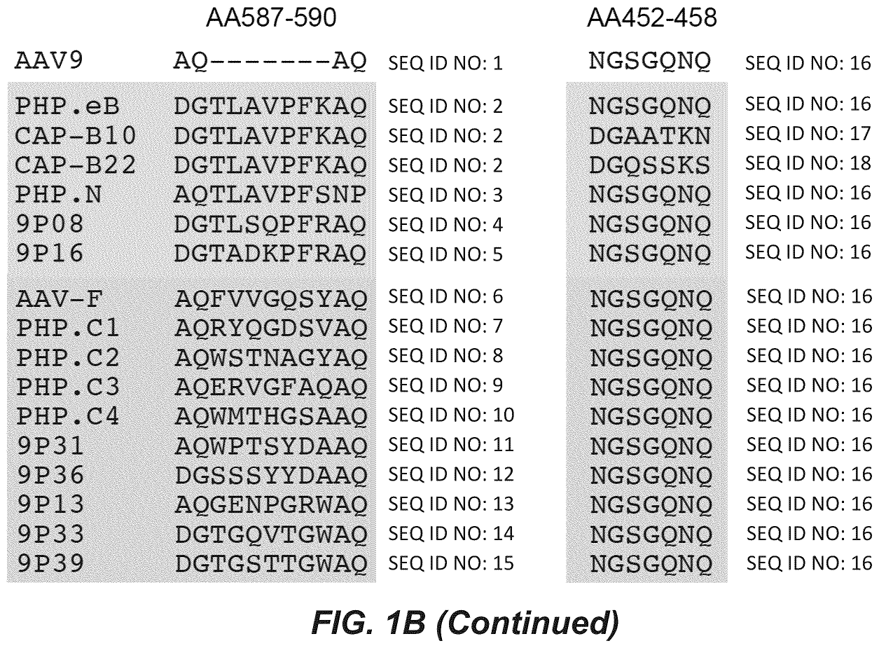

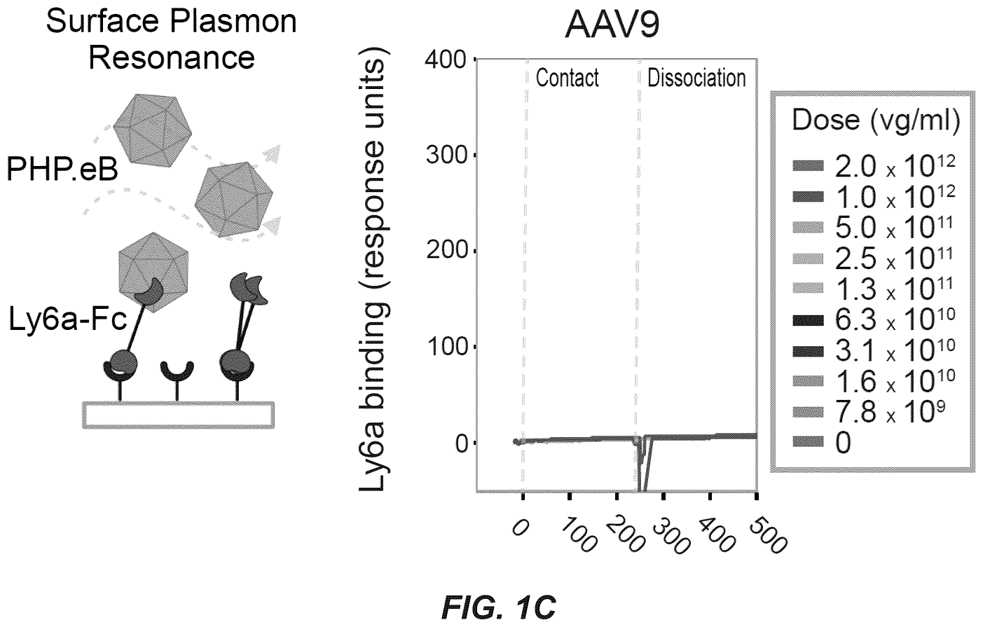

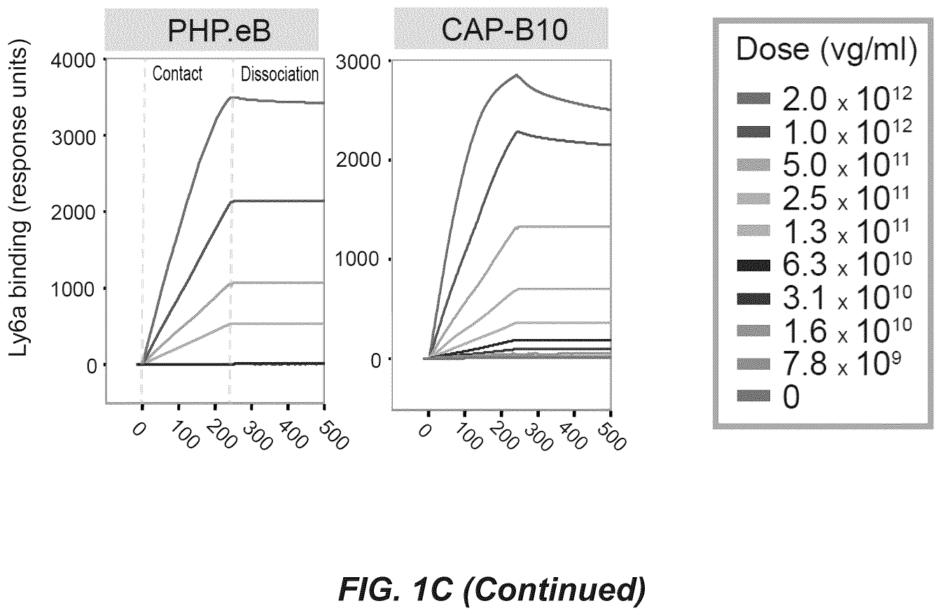

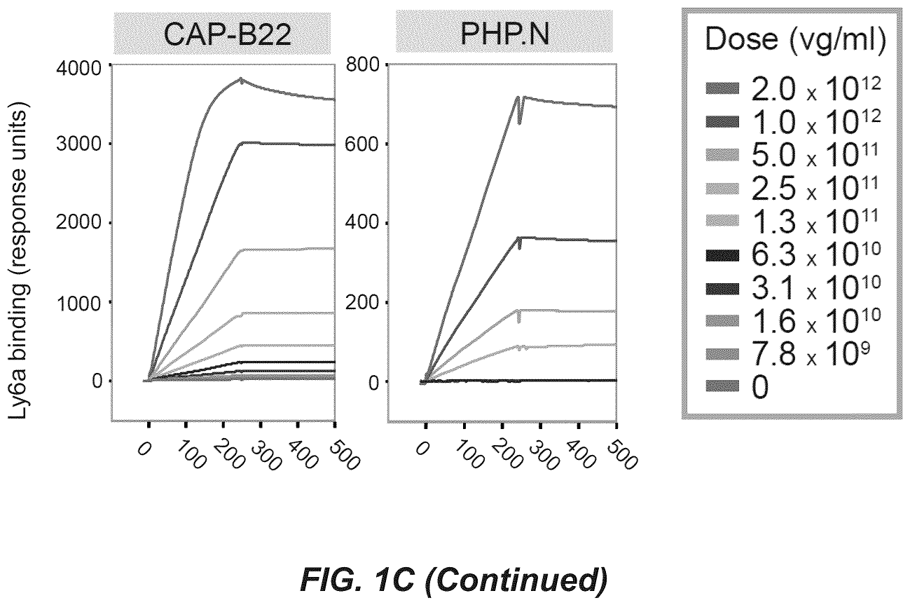

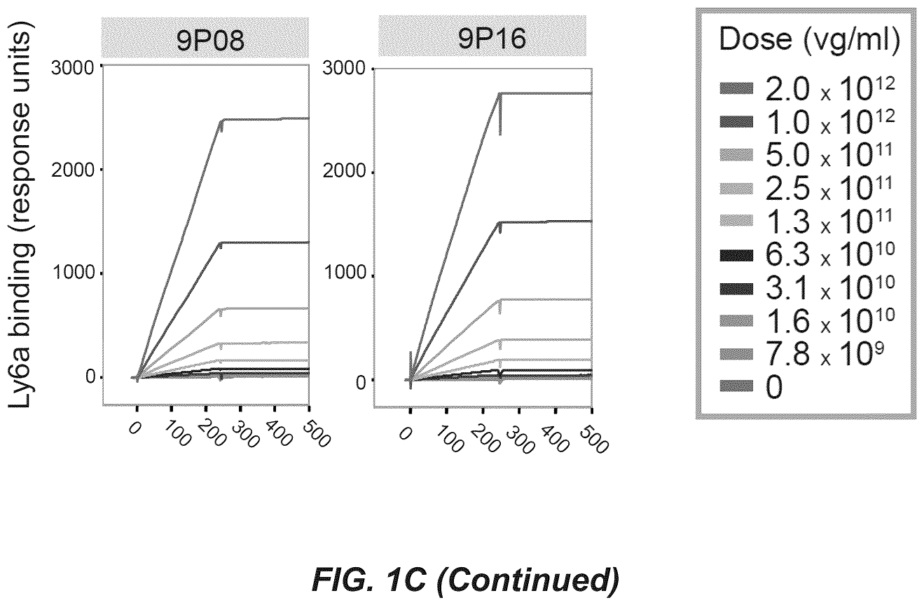

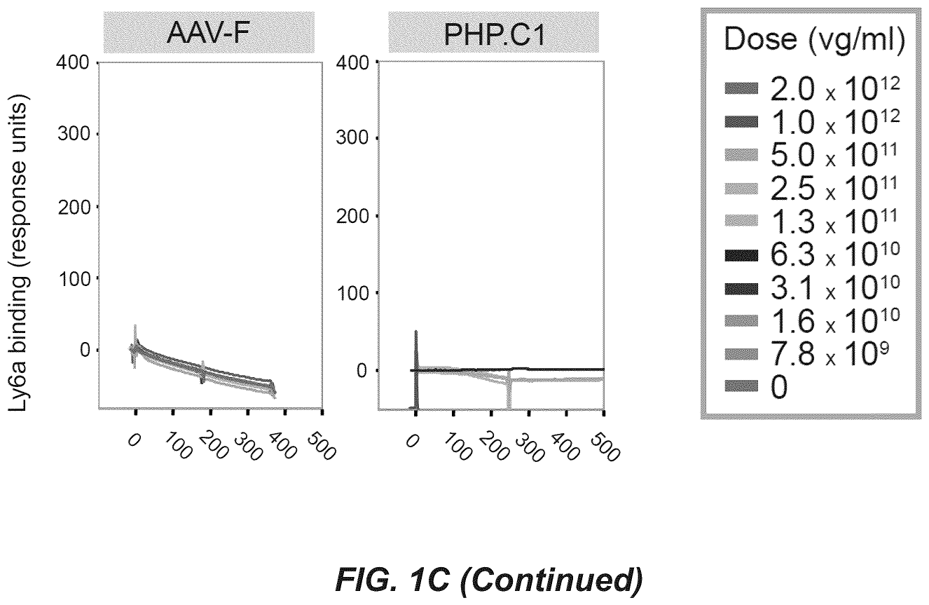

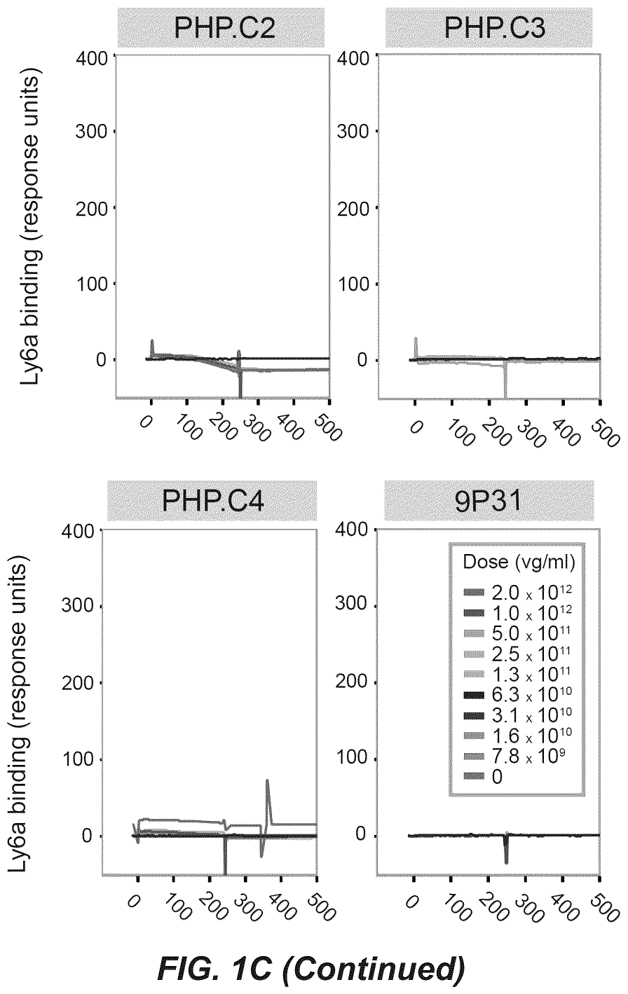

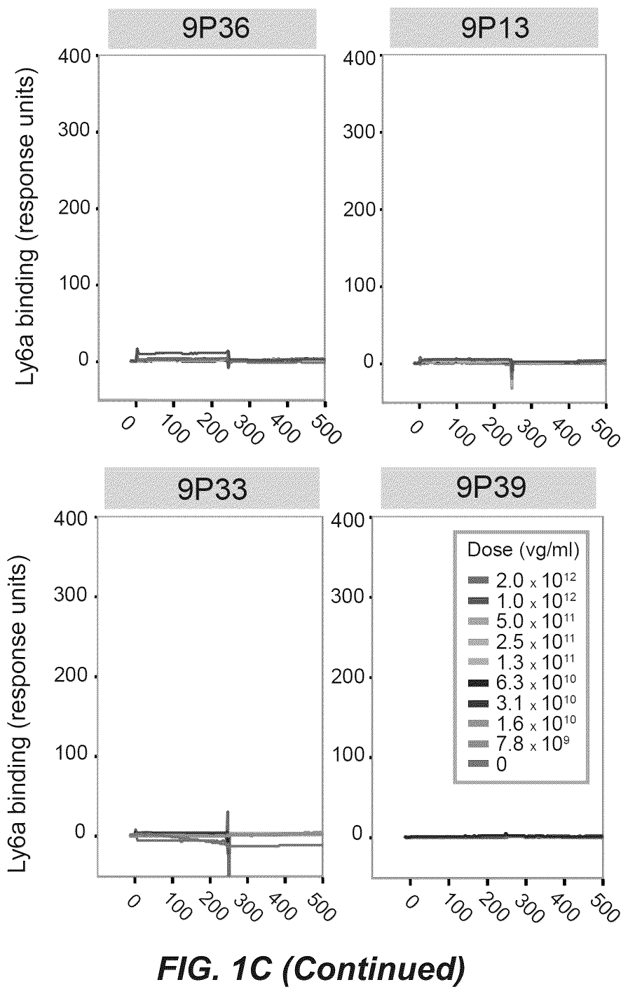

Ly6a is the receptor responsible for the enhanced CNS tropism of PHP.B and PHP.eB in mice. One strain-specific SNP disrupts its GPI-anchored membrane localization (FIG. 1A). Previously, M-CREATE directed evolution selection platform was applied to a library of AAV9 variants (containing seven-amino-acid insertions at position 588 of capsid variable region VIII), identifying a family of engineered AAVs with diverse CNS tropisms and a shared sequence motif, typified by their founding member, AAV-PHP.B (FIG. 1B). The variants' enhanced CNS potency was lacking in BALB/cJ mice, a phenomenon explained by their shared reliance on Ly6a for BBB crossing. Recently, multiple engineered AAVs outside the PHP.B sequence family, which retained their enhanced CNS tropism in BALB/cJ mice were identified. Before attempting to de-orphanize these AAVs, their independence of Ly6a was first confirmed.

To directly probe potential Ly6a binding interactions, we performed surface plasmon resonance (SPR) was performed. To ensure the detection of even weak interactions, Ly6a was dimerized by fusion to Fc and immobilized at high density on a protein A chip. Each AAV analyte was tested across a range of concentrations (FIG. 1C). As expected, AAV9 showed no evidence of binding at any concentration, and PHP.B sequence family members all showed strong binding interactions with Ly6a. While precise affinities could not be determined due to the effects of avidity and mass transport, the interaction profiles were consistent with sub-nanomolar affinities. Conversely, all BALB/cJ-enhanced engineered AAVs were indistinguishable from AAV9, exhibiting no detectable interaction with Ly6a.

### CNS Potency of Ly6a-Independent Engineered AAVs Across Genetically Diverse Mouse Strains

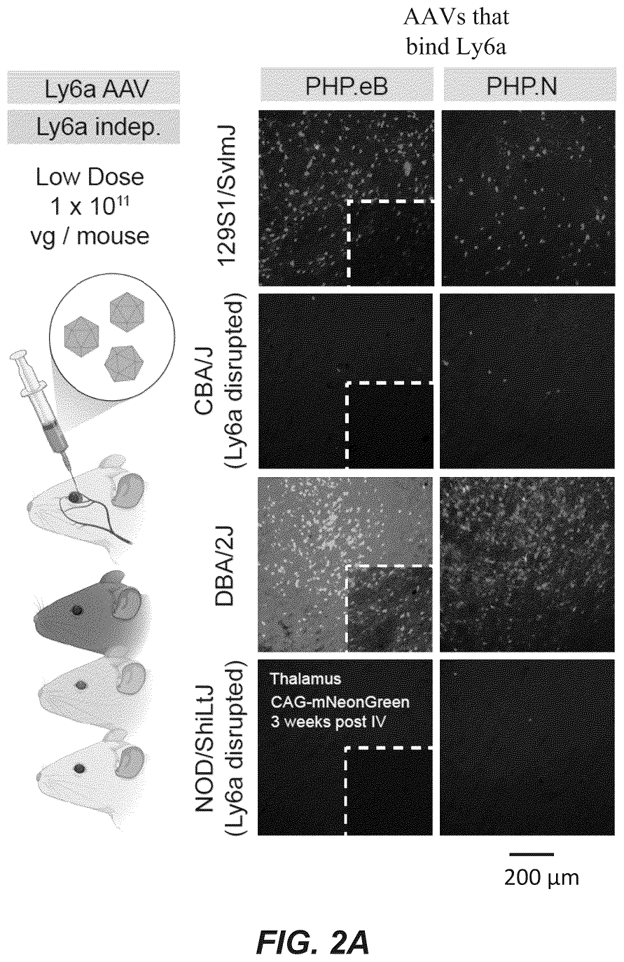

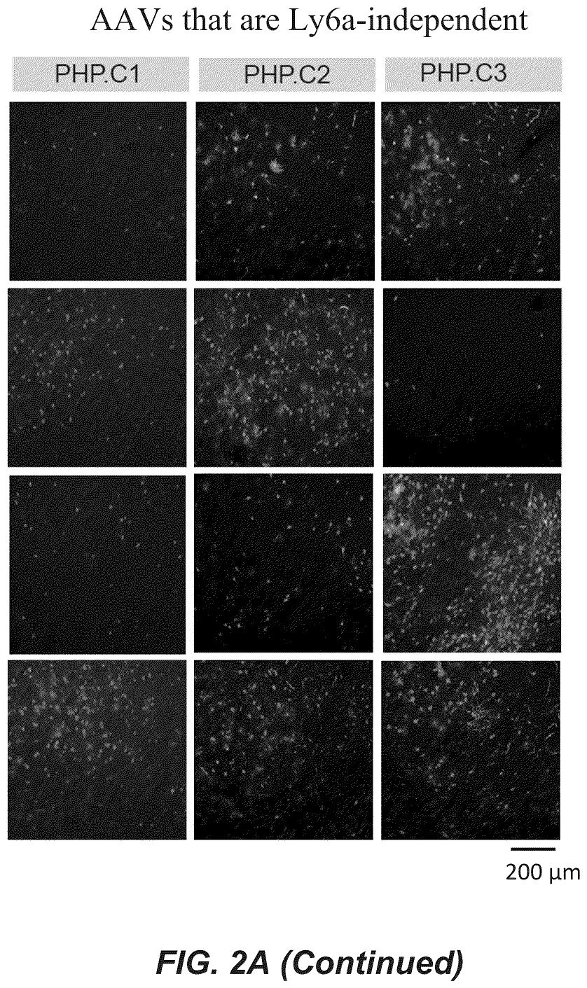

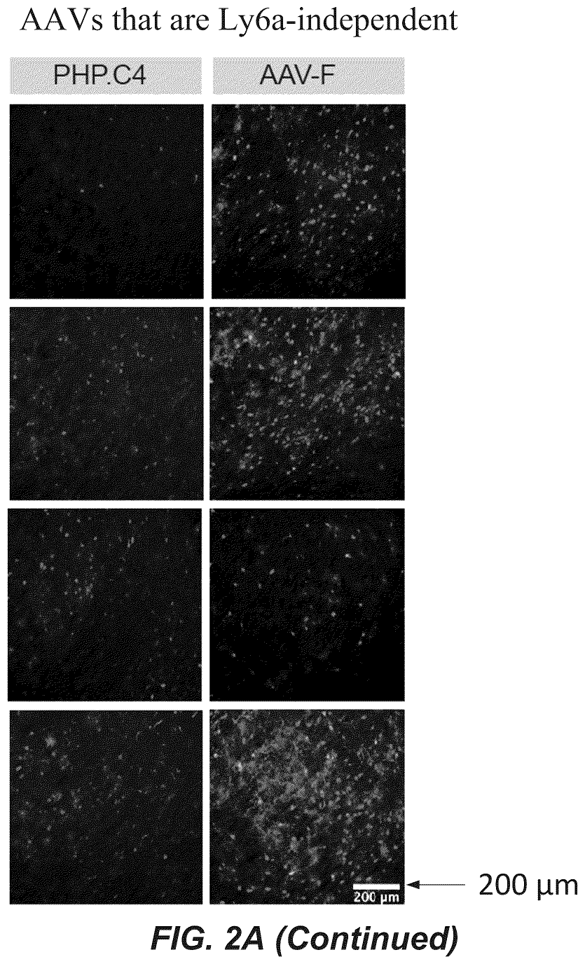

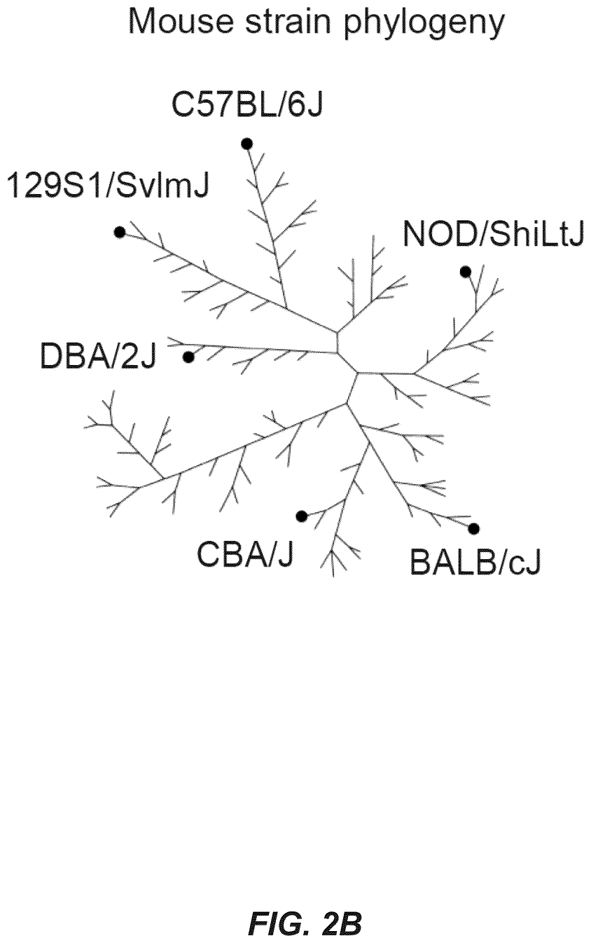

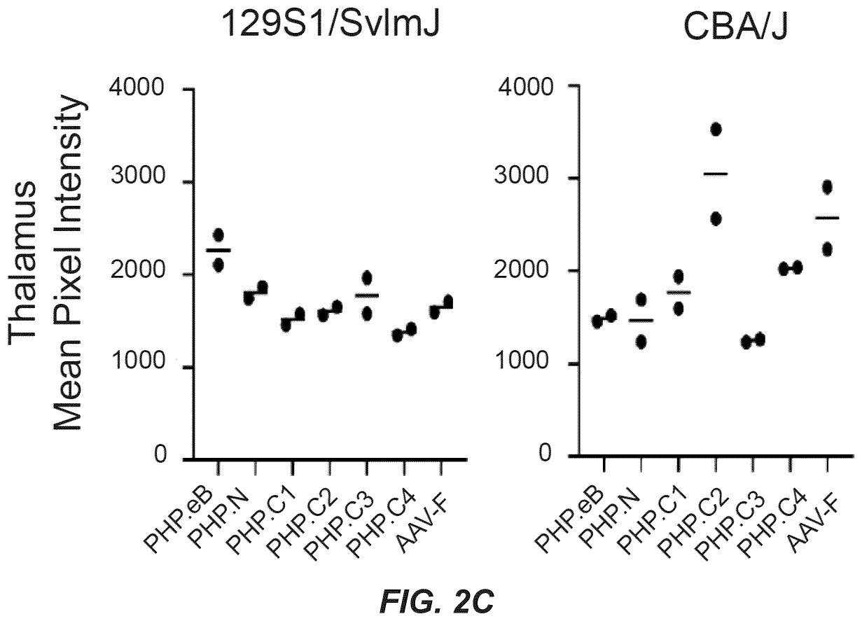

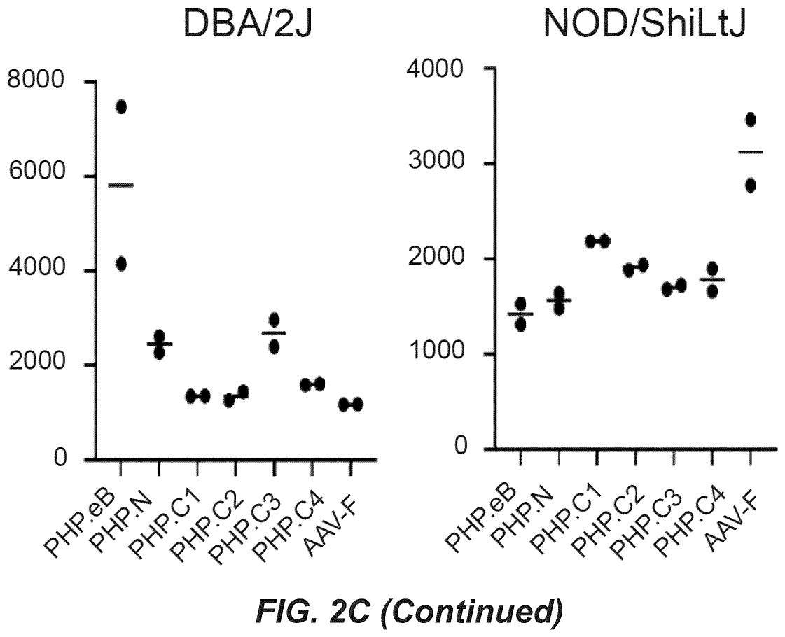

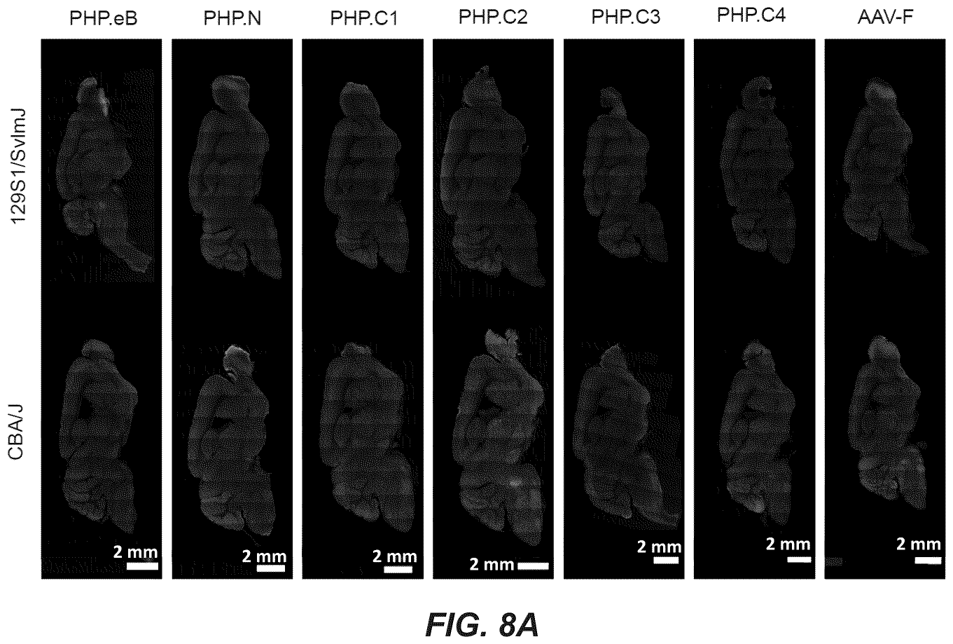

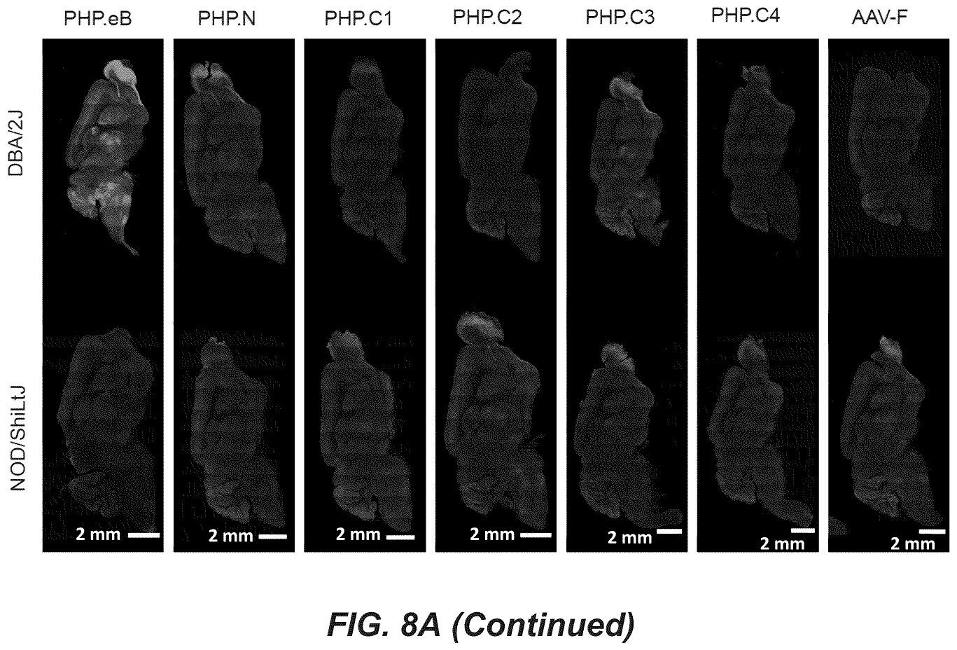

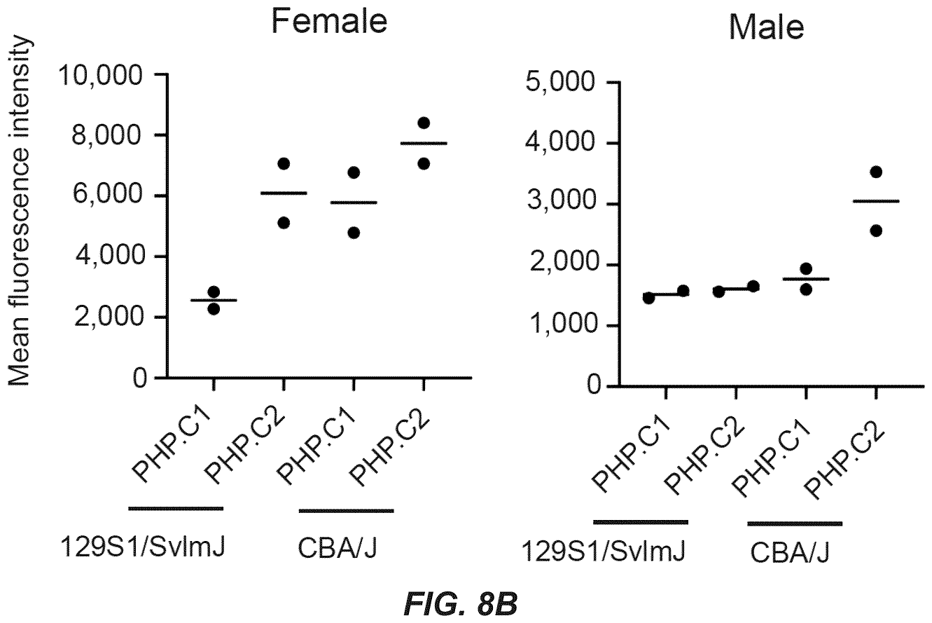

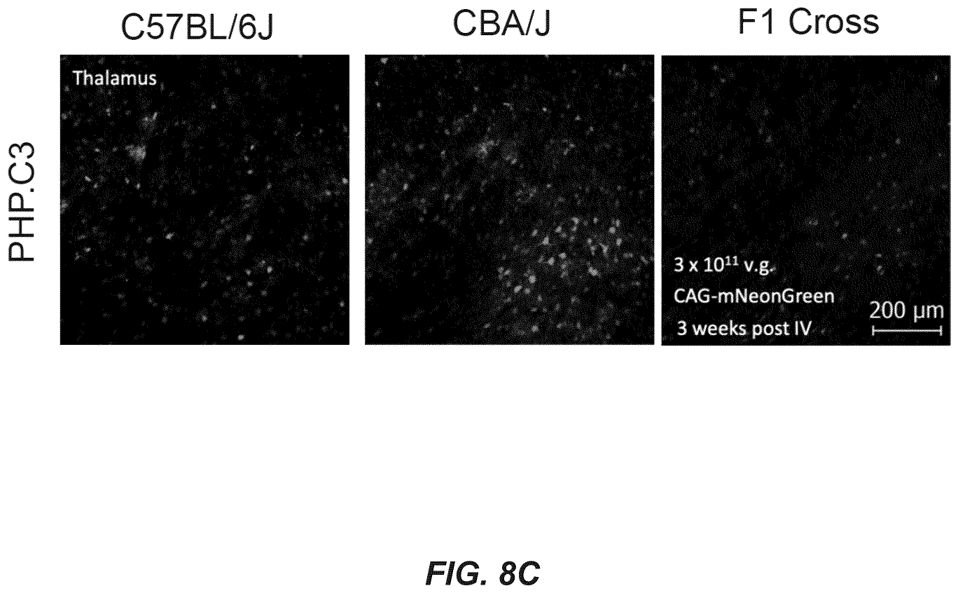

To characterize strain-specific differences in the CNS potency of BALB/cJ-enhanced AAVs, an exploratory assessment was performed with several of them in multiple genetically-diverse mouse strains (FIG. 2A-FIG. 2C and FIG. 8A). ssAAVs (ss, single-stranded) packaging CAG-mNeonGreen were retro-orbitally injected at a low dose of 1×10¹¹ v.g. per animal. Tissues were harvested and imaged 3 weeks post-injection. The thalamus was chosen for comparison as this brain region was potently targeted by all AAVs tested. As expected, given their Ly6a interaction, PHP.eB and PHP.N demonstrated enhanced potency in 129S1/SvlmJ and DBA/2J mouse strains, which expressed membrane-localized Ly6a, and greatly reduced potency in CBA/J and NOD/ShiLtJ mouse strains, which expressed GPI-disrupted Ly6a. Ly6a-independent AAVs, however, showed a distinct pattern of enhanced infectivity. Selective AAV and mouse strain combinations were probed in both sexes, with similar trends across strains observed (FIG. 8B). The potentially higher potency observed in female mice can result from AAV batch-to-batch variability or sex-linked differences in AAV potency.

Previously, genetic techniques were used to identify the Ly6a receptor. Following this approach to identify the receptor responsible for enhanced CNS transduction of Ly6a-independent vectors, C57BL/6J and CBA/J mice were crossed and the vector PHP.C3 was tested in their offspring (F1), expecting an intermediate phenotype if the receptor was inherited akin to Ly6a. Both parental strains and F1 adults were retro-orbitally injected with 3×10¹¹ v.g. per animal. After 3 weeks, tissue was harvested and imaged. Surprisingly, at this higher dose, PHP.C3 demonstrated robust CNS infectivity in all three populations (FIG. 8C). This dose dependence suggested a subtler mechanistic difference from the presence or absence of a single receptor, which led to the application of a different approach.

### Construction of a Cell Culture Screen for Putative Receptors of Engineered AAVs

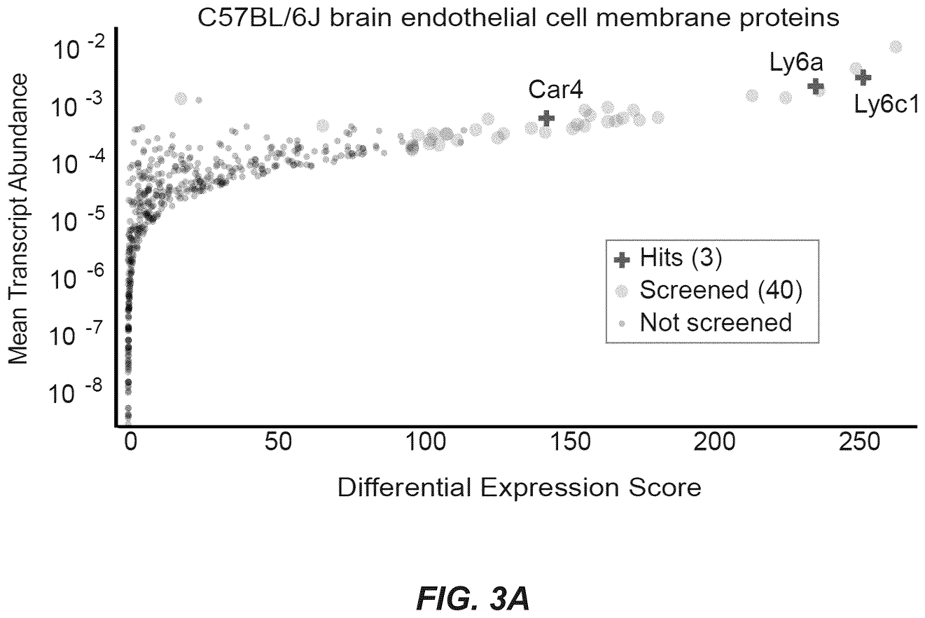

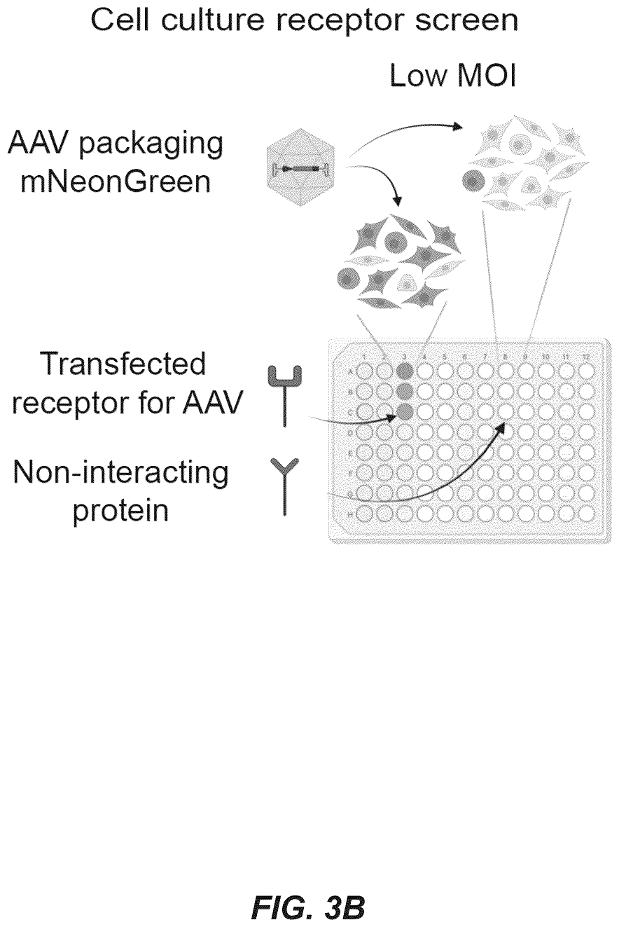

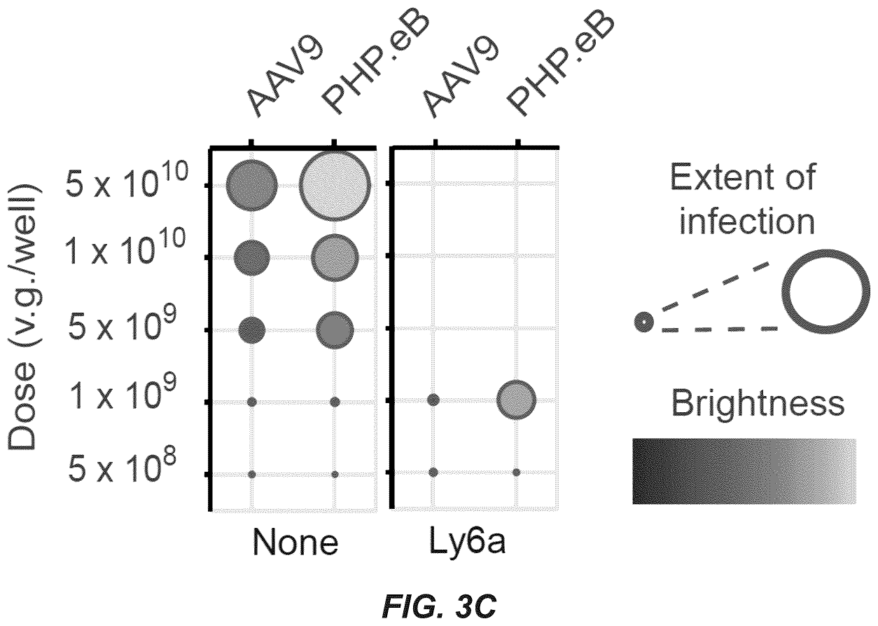

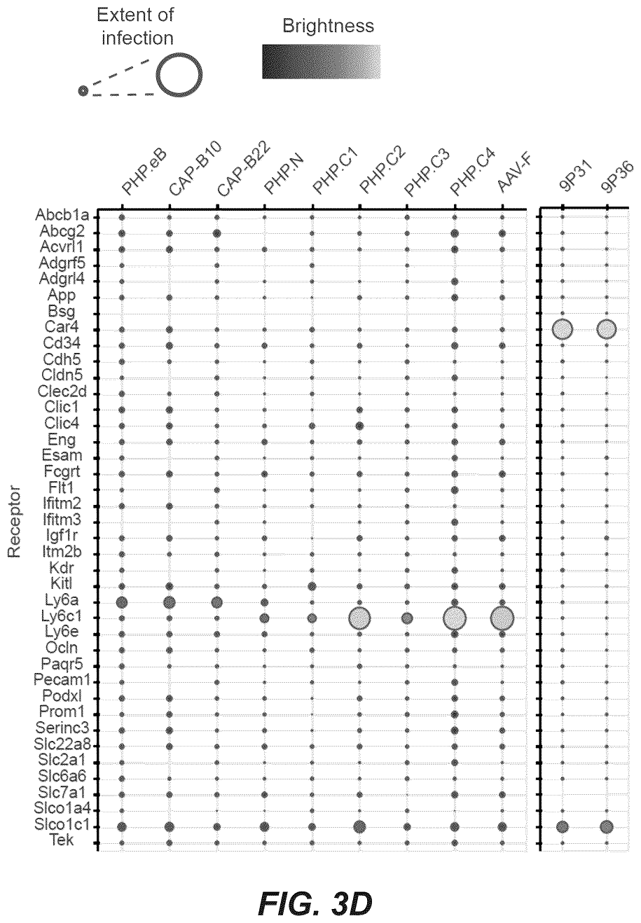

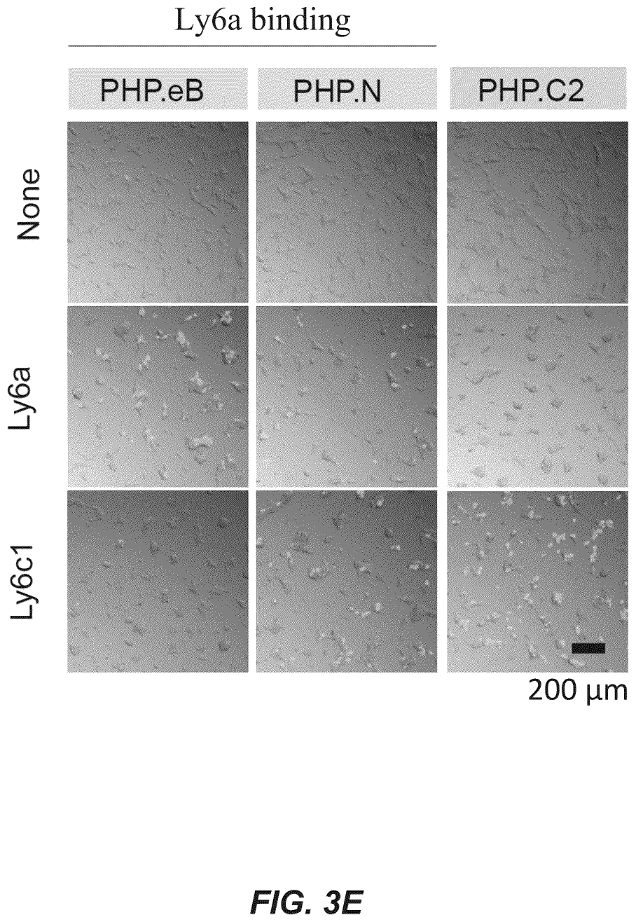

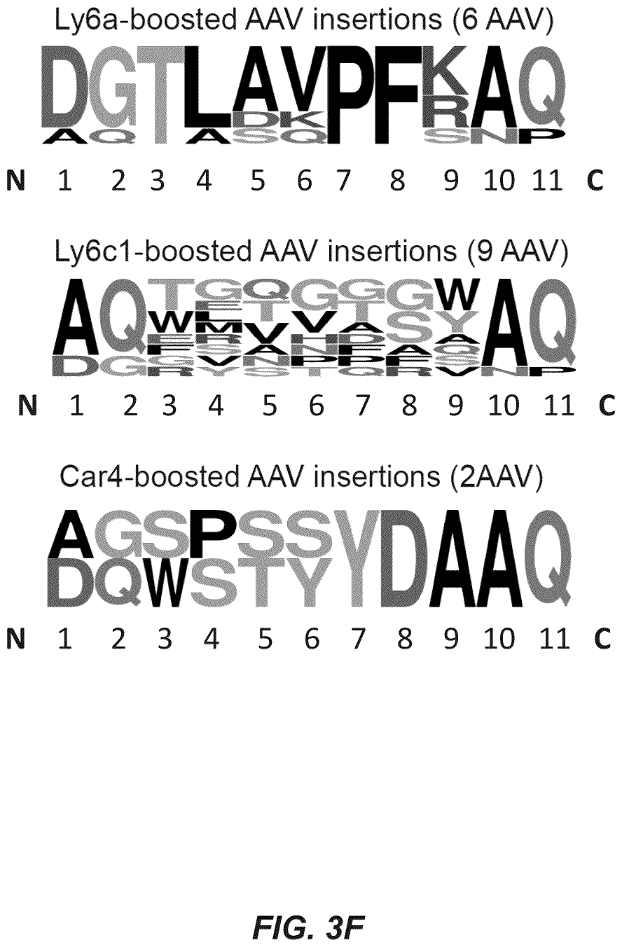

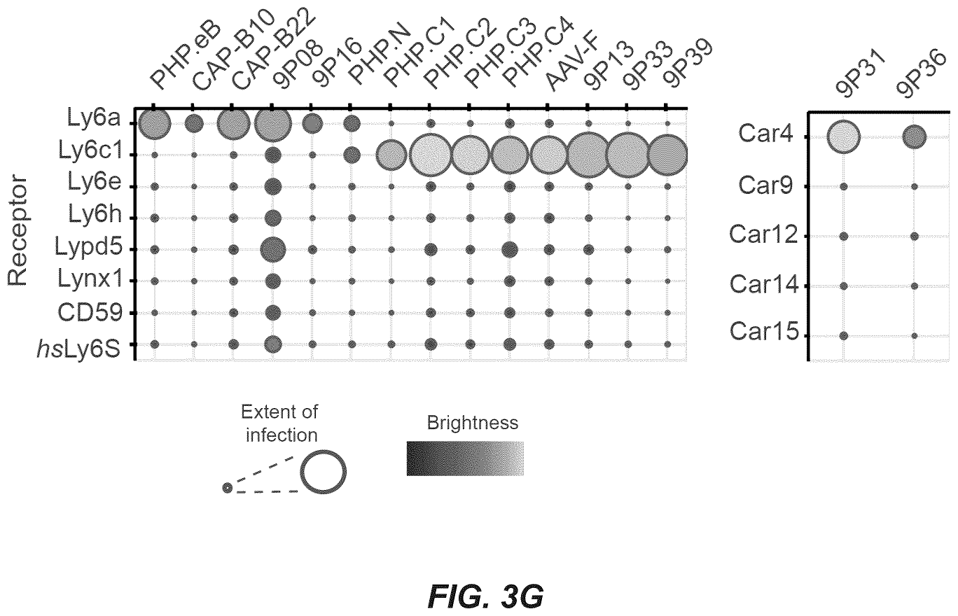

A receptor, for which engineered AAVs can co-opt efficient BBB crossing, is likely to be both highly expressed and highly specific to the endothelial cells of the brain. Thus, previously-collected single-cell RNA sequencing data from dissociated C57BL/6J brain tissue was analyzed (FIG. 3A). Gene expression levels and differential expression compared to all other CNS cell type clusters were investigated within CNS endothelial cell clusters. Genes were filtered to select only those annotated in Uniprot as localized to the plasma membrane before calculating their endothelial cell differential expression scores in Scanpy. These scores were then plotted against each transcript's mean abundance, revealing a long tail of highly expressed and highly specific CNS endothelial membrane proteins. Encouragingly, Ly6a appeared at the far reach of this tail. From this analysis, a panel of 40 abundant and specific candidate receptors was selected (Table 3).

TABLE 3
Single-cell RNA sequencing profiles of the C57bL/6J brain endothelial
cell membrane proteins included in cell culture screen

| | | Mean | Differential |
|---|---|---|---|
| Name | Accession No. | Expression | Expression |
| Abcb1a | NM 011076.2 | 0.000688904 | 7.942371 |
| Abcg2 | NM 011920.3 | 0.000745241 | 5.5607147 |
| Acvrl1 | NM 009612.3 | 0.000485079 | 6.7861333 |
| Adgrf5 | NM_001357332.1 | 0.000546496 | 7.453164 |
| Adgrl4 | NM_133222.3 | 0.000763887 | 8.241394 |
| App | NM_001198823.1 | 0.000845707 | 0.04600084 |
| Bsg | NM_009768.2 | 0.013027801 | 4.6841507 |
| Car4 | NM_007607.2 | 0.000713242 | 3.7107854 |
| Cd34 | NM_133654.3 | 0.000467251 | 3.0983913 |
| Cdh5 | NM_009868.4 | 0.000332645 | 7.486805 |
| Cldn5 | NM_013805.4 | 0.005443705 | 9.174832 |
| Clec2d | NM_053109.3 | 0.000584914 | 6.370406 |
| Clic1 | NM_033444.2 | 0.000377934 | 2.6402416 |
| Clic4 | NM_013885.2 | 0.000408817 | 3.1440303 |
| Eng | NM_007932.2 | 0.000646813 | 5.9965453 |
| Esam | NM_027102.3 | 0.000660755 | 6.0463476 |
| Fcgrt | NM_010189.3 | 0.000503876 | 4.028085 |
| Flt1 | NM_010228.3 | 0.002267079 | 8.539886 |
| Ifitm2 | NM_030694.1 | 0.000314049 | 3.5160391 |
| Ifitm3 | NM_025378.2 | 0.001015794 | 5.313354 |
| Igf1r | NM_010513.2 | 0.000750016 | 3.374137 |
| Itm2b | NM_008410.2 | 0.001596154 | −0.10450481 |
| Kdr | NM_010612.2 | 0.000304256 | 6.8344116 |
| Kitl | NM_013598.3 | 0.000382663 | 3.2804174 |
| Ly6a | NM_010738.3 | 0.002695857 | 8.700766 |
| Ly6c1 | NM_010741.3 | 0.00381227 | 8.841889 |
| Ly6e | NM_008529.3 | 0.001050145 | 2.9547617 |
| Ocln | NM_008756.2 | 0.000208863 | 6.836091 |
| Paqr5 | NM_028748.2 | 0.000262998 | 7.7742887 |
| Pecam1 | NM_008816.3 | 0.000423881 | 7.1343536 |
| Podxl | NM_013723.3 | 0.000407256 | 6.3635473 |
| Prom1 | NM_001163577.1 | 0.000271125 | 5.4568763 |
| Serinc3 | NM_012032.4 | 0.000562215 | 1.2317827 |
| Slc22a8 | NM_001164634.1 | 0.000243451 | 5.054169 |
| Slc2a1 | NM_011400.3 | 0.001843255 | 5.4381437 |
| Slc6a6 | NM_009320.4 | 0.001148025 | 3.8466463 |
| Slc7a1 | NM_007513.4 | 0.000403344 | 4.1706085 |
| Slco1A4 | NM_030687.1 | 0.001701704 | 8.396132 |
| Slco1c1 | NM_021471.2 | 0.000828427 | 3.688315 |
| Tek | NM_013690.3 | 0.000224058 | 7.496325 |

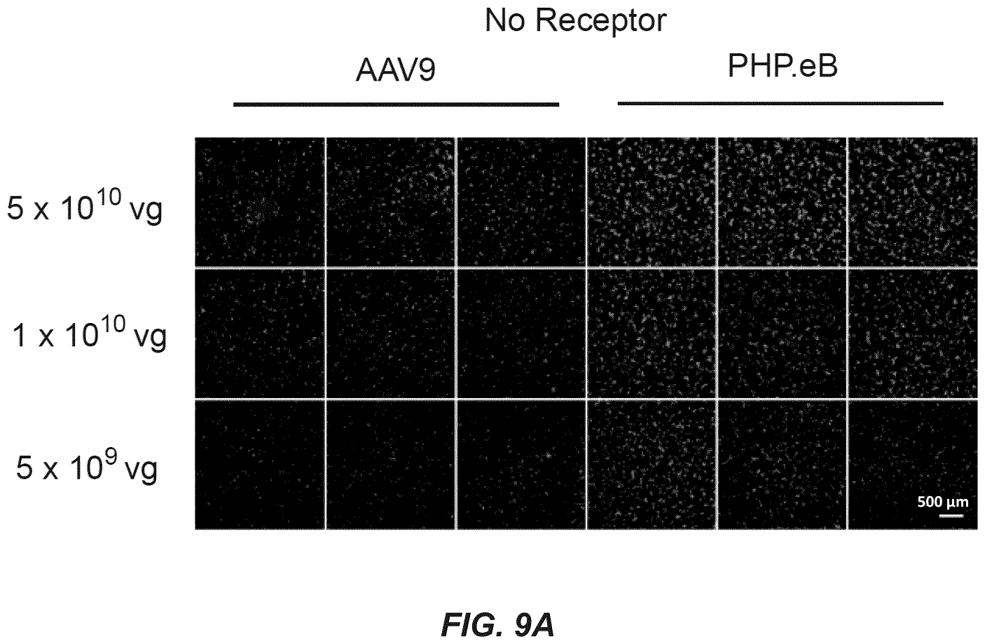

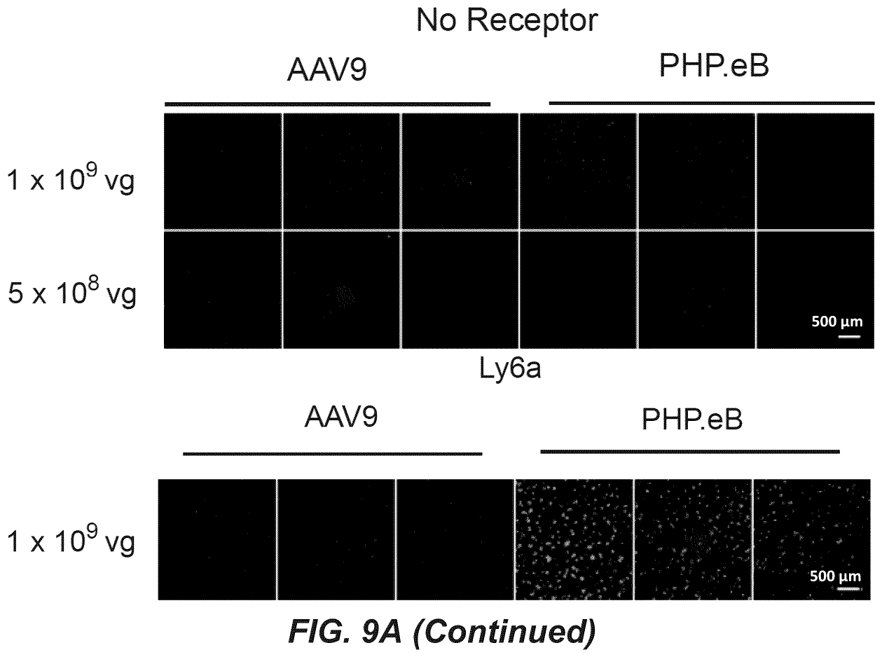

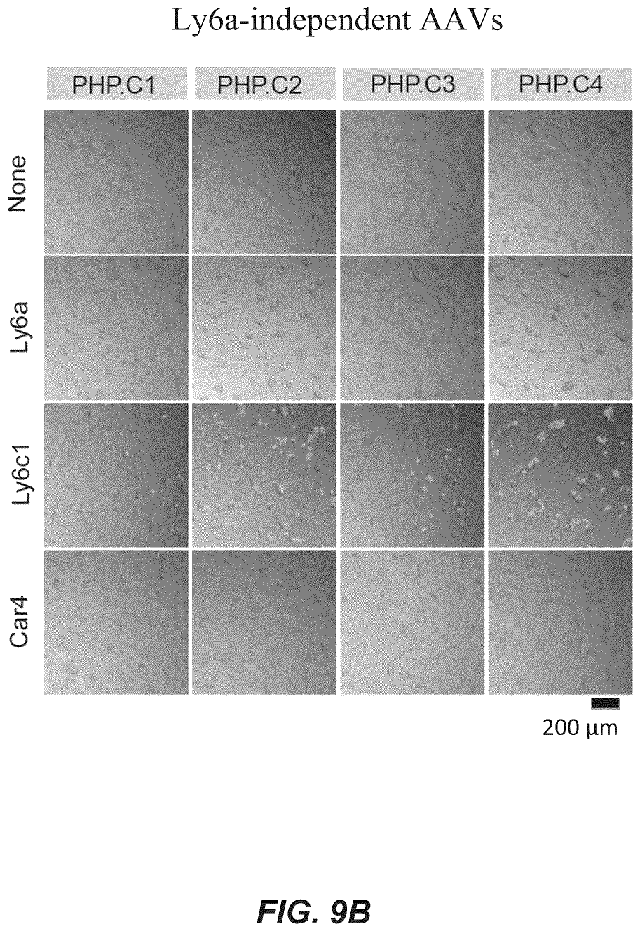

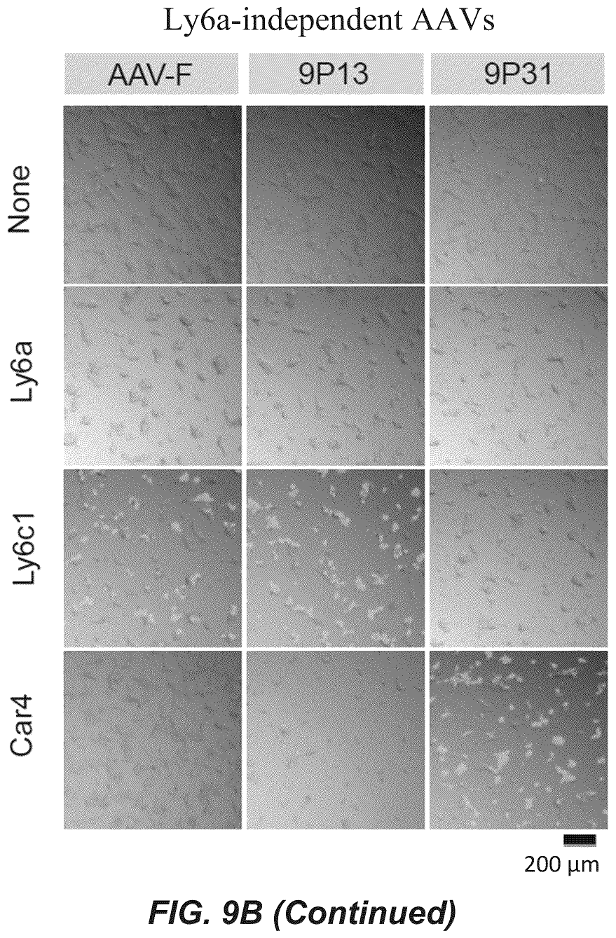

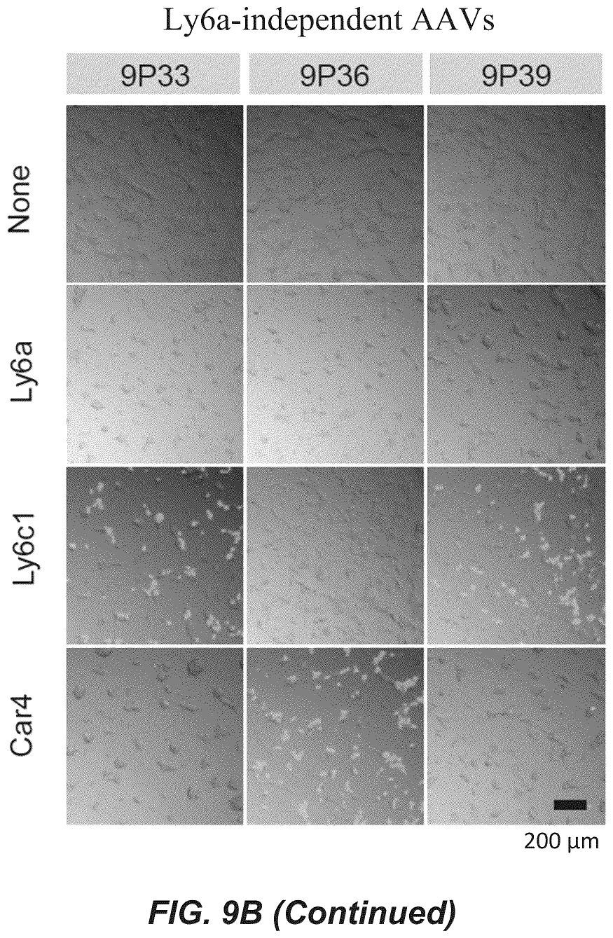

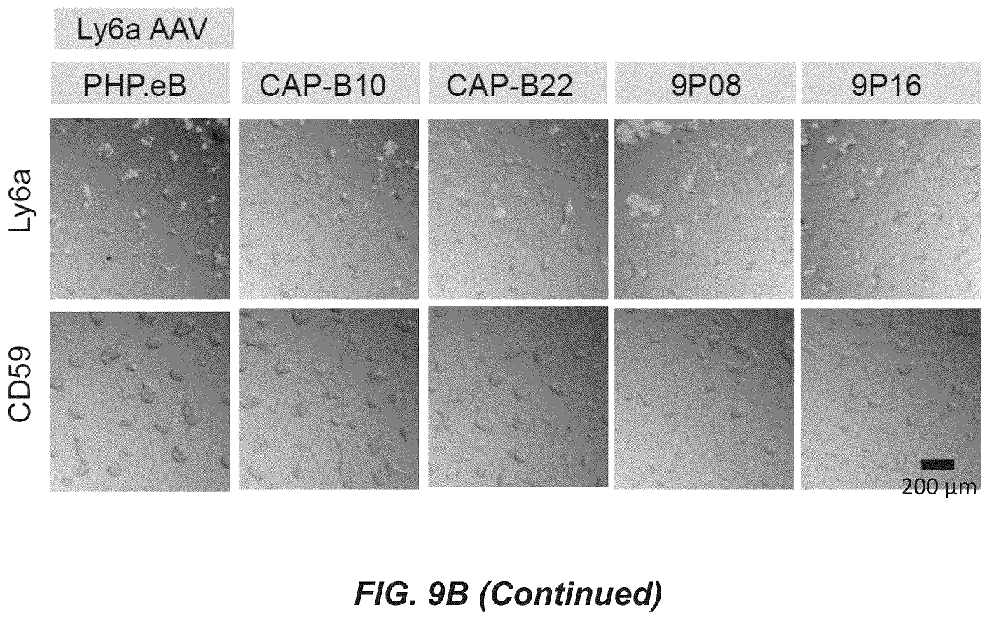

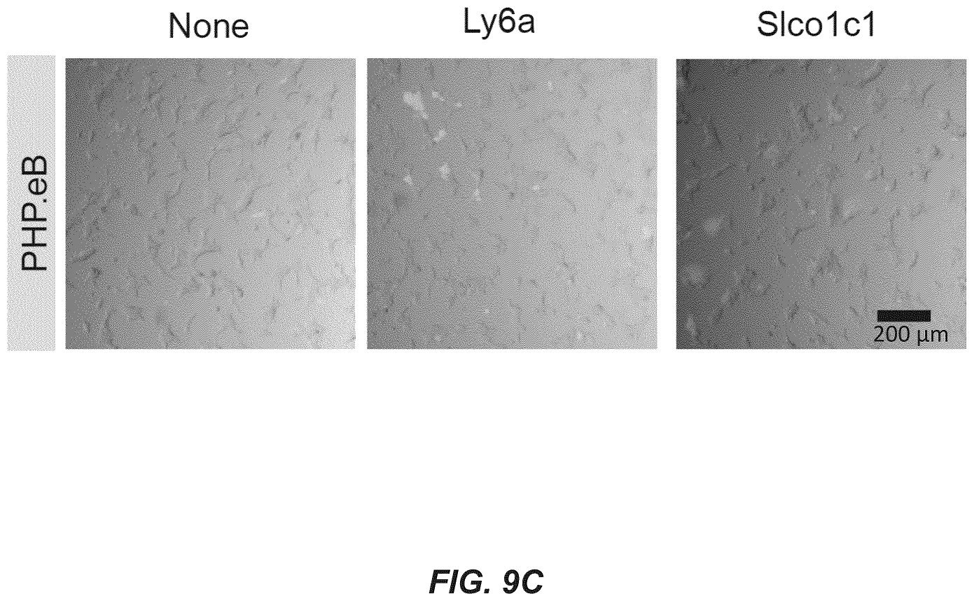

It has been observed that expression of membrane-localized Ly6a in HEK293 cells selectively improved the potency of PHP.eB infection compared to AAV9 at low multiplicity of infection (MOI), with the extent of infection and brightness of infected cells remarkably increased (FIG. 9A). This property is likely to be conserved among BBB-crossing receptors. Thus, this behavior was made the basis of a receptor transient overexpression screening (FIG. 3B and FIG. 3C). The C57BL/6J coding sequences of each of the 40 candidate receptors were cloned into a mammalian expression plasmid and tested against engineered AAVs in triplicate at two different doses (FIG. 3A and FIG. 3D). PHP.eB paired with Ly6a served as a positive control.

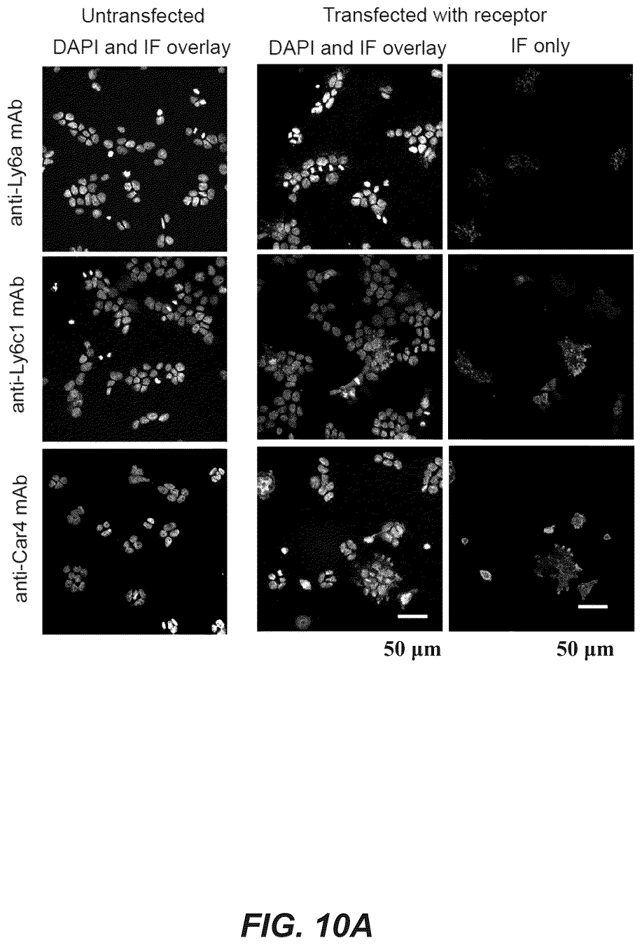

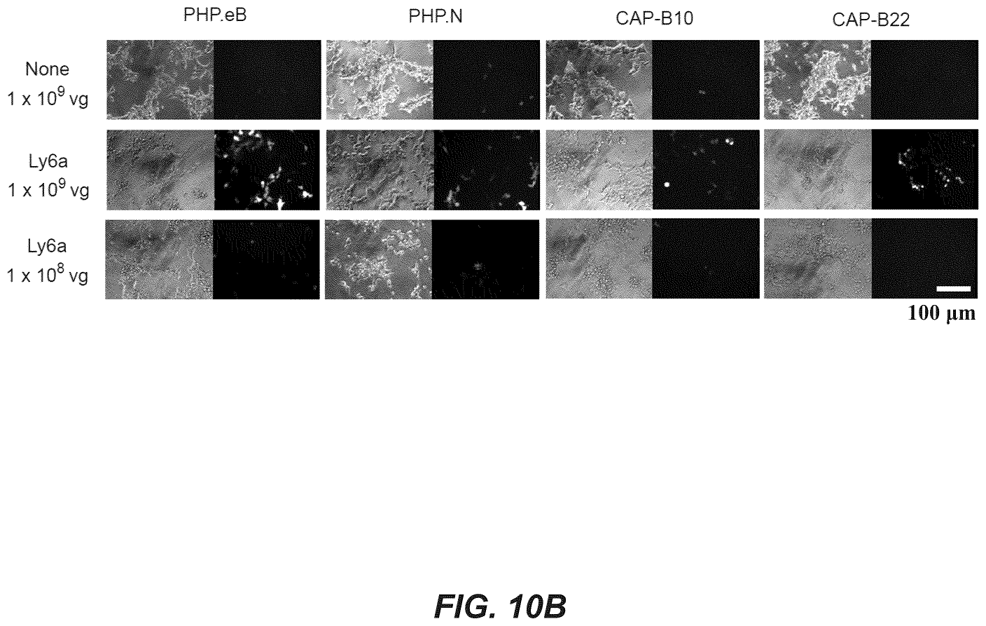

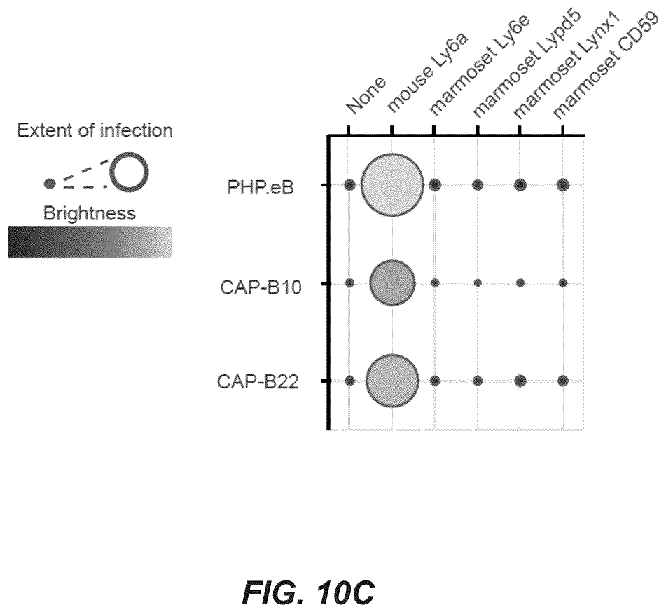

As expected, all members of the PHP.B sequence family showed a remarkable boost in infectivity in Ly6a-transfected cells compared to untransfected cells, while Ly6a-independent AAVs performed identically under both conditions (FIG. 3D, FIG. 3E, and FIG. 9B). Interestingly, the infectivity of all Ly6a-dependent capsids was boosted to a similar extent (FIG. 10B).

### Identification of Ly6c1 and Car4 as Receptors for BBB Crossing of Ly6a-Independent Engineered AAVs

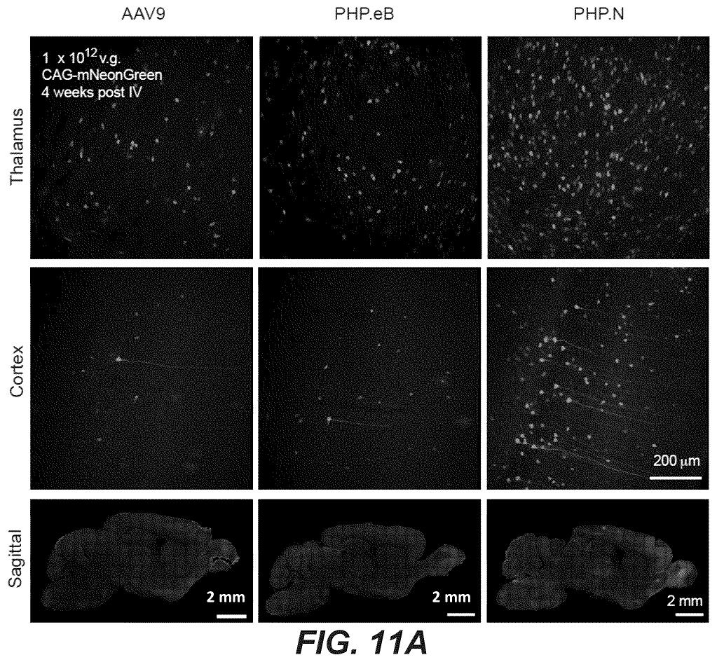

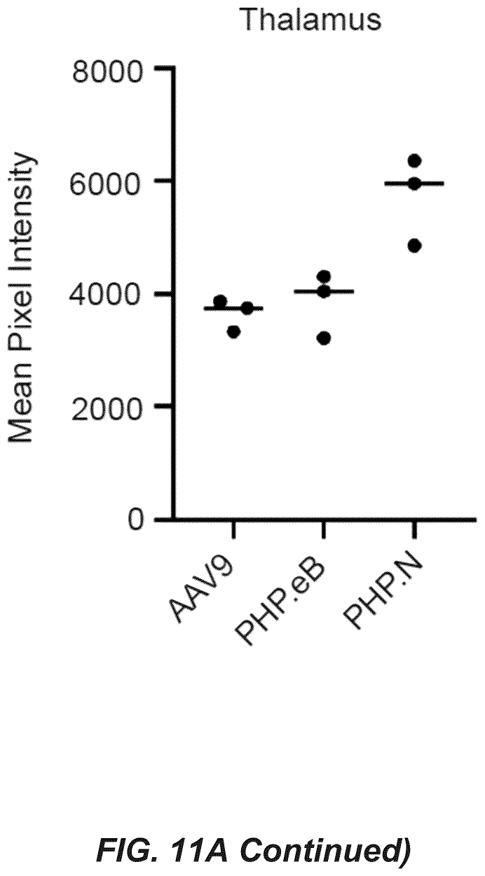

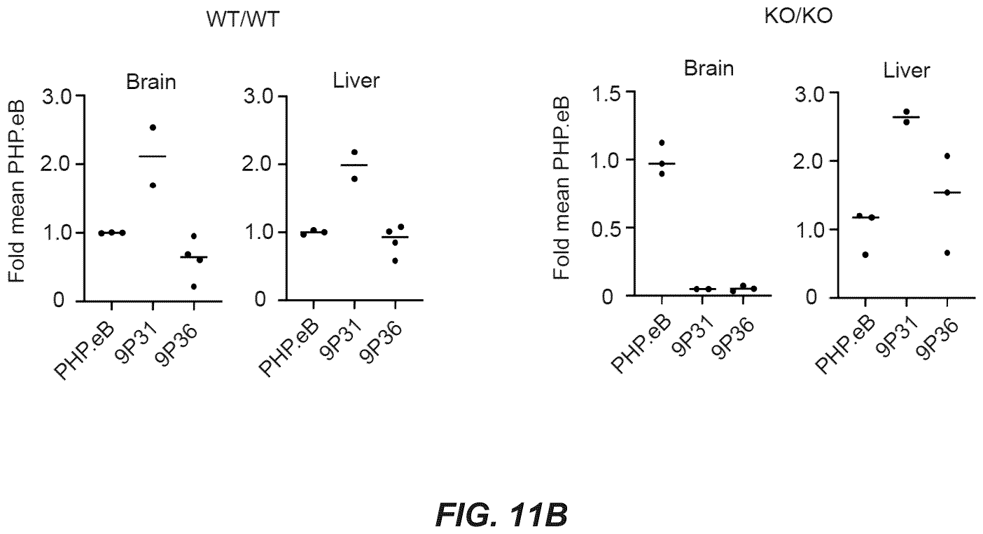

Boosts in infectivity for all tested Ly6a-independent AAVs with novel receptors (FIG. 3D) were observed. Surprisingly, given their different sequence families and strain-dependent patterns of CNS potency at low dose, all of the initial Ly6a-independent AAVs responded to the same candidate receptor, Ly6c1 (FIG. 3D-FIG. 3G and FIG. 9B). In addition, despite its Ly6a-dependent pattern of CNS infectivity across murine strains, PHP.N also exhibited enhanced infectivity in Ly6c1-transfected cells. At a higher dose, PHP.N outperformed both AAV9 and PHP.eB in CBA/J mice (FIG. 11A and FIG. 11B), which expressed a GPI-disrupted Ly6a. While polymorphisms in Ly6c1 existed among mouse strains (as shown by a published analysis of the 36 mouse strains, for which whole genomes were available), none of these polymorphisms correlated with the pattern of CNS potency of any of the eight Ly6c1-interacting AAVs. Additionally, unlike Ly6a, none of the polymorphisms were predicted to disrupt GPI-anchoring of Ly6c1 to the plasma membrane using PredGPI. To determine the specificity of the Ly6c1 interaction, a follow-up screening was performed with additional closely-related and CNS-expressed Ly6 superfamily members. No cross-reactivity of Ly6c1-dependent AAVs with other Ly6 proteins was found (FIG. 3G). CAP-B10 and CAP-B22, which have seven amino acid substitutions in capsid variable region IV and showed enhanced potency in adult marmosets, did not exhibit any additional receptor interactions in either of the screenings described above or a third screening with marmoset CNS-expressed Ly6 family members (FIG. 10C) that would explain their NHP tropism. Moreover, the Ly6-interacting AAVs did not cross-react with the recently described human Ly6S, a close relative of murine Ly6a (FIG. 3G).

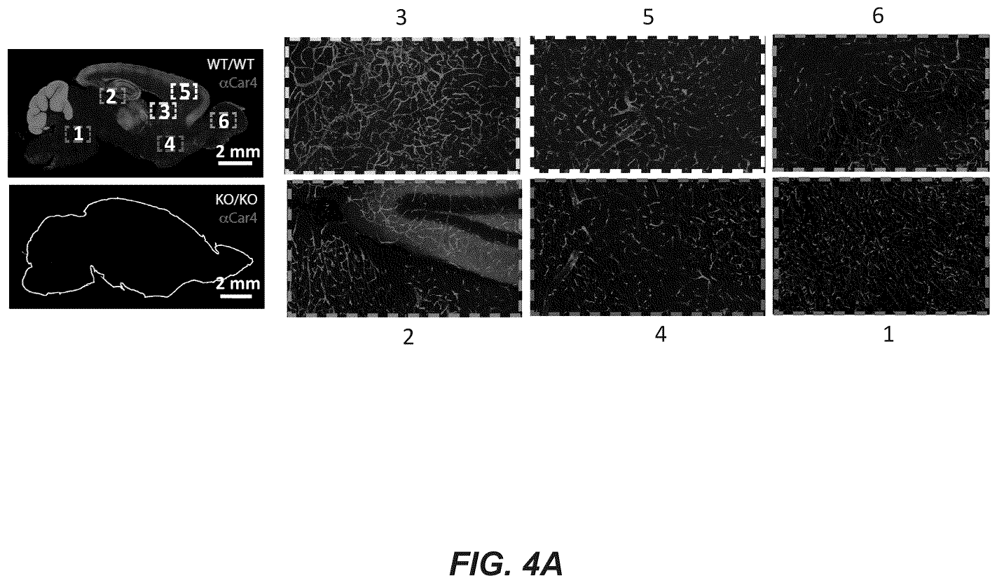

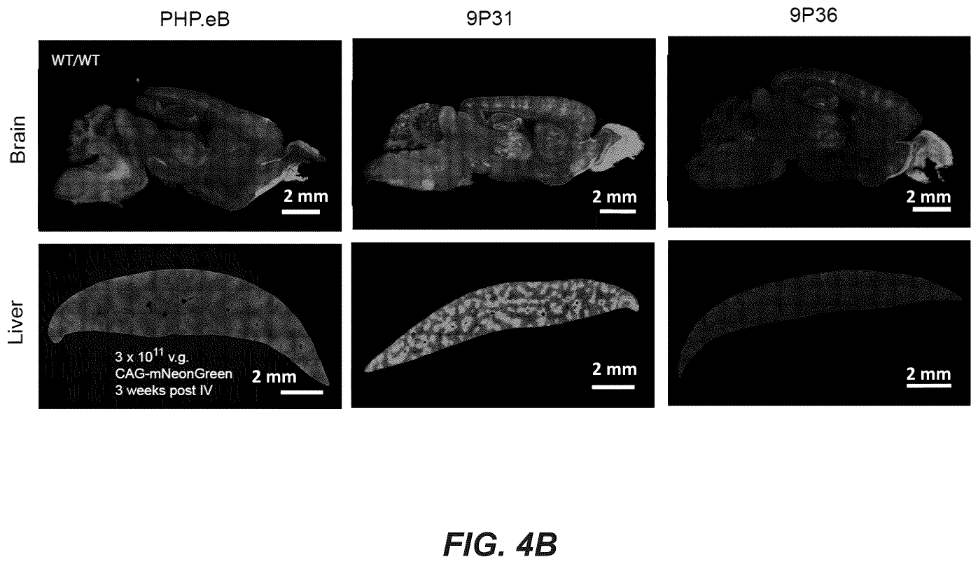

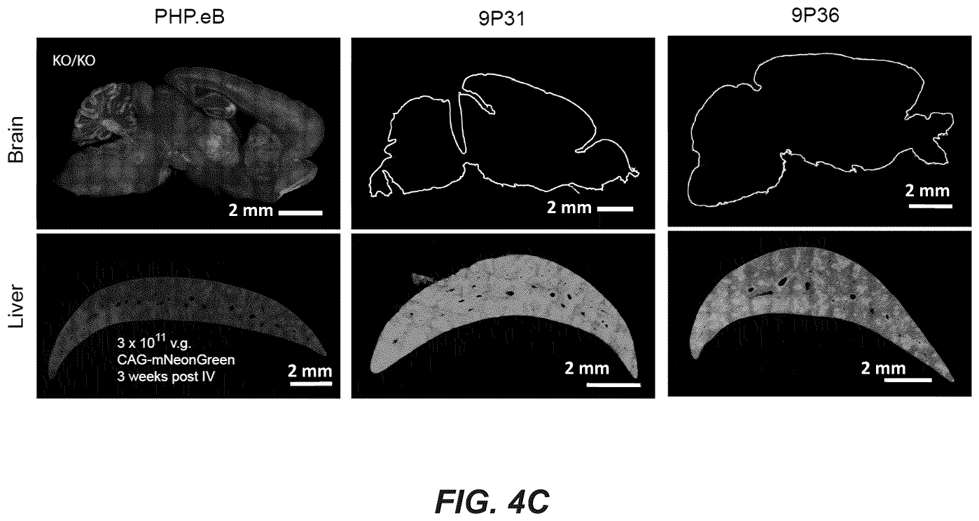

The Ly6 follow-up screening was expanded to include a second set of previously identified engineered AAV (9P), which yielded additional Ly6a and Ly6c1-interacting AAVs (FIG. 3G). 9P31 and 9P36, however, did not display enhanced infectivity with any Ly6 proteins in the panel and, therefore, were tested on the full receptor screening (FIG. 3D). Both 9P31 and 9P36 displayed an infectivity boost with the GPI-linked enzyme Car4 (FIG. 3D and FIG. 9B). This interaction was specific to Car4 among membrane-associated carbonic anhydrases (FIG. 3G). As with Ly6c1, polymorphisms in Car4 across mouse strains were not predicted to impact GPI-anchoring to the plasma membrane. Unlike mouse-restricted Ly6a and Ly6c1, Car4 is conserved throughout vertebrates, including non-human primates and humans. Therefore, the screening results were confirmed in vivo using Car4-knockout mice (B6.129S1-Car4^{tm1Sly}/J, Jackson labs strain #008217). Immunofluorescence confirmed that Car4 was strongly expressed throughout the brain vasculature of homozygous WT/WT mice and completely absent in KO/KO (FIG. 4A). When dosed with 3×10¹¹ v.g. per animal, PHP.eB, 9P31, and 9P36 all strongly expressed in both the brain and liver of wild-type mice (FIG. 4B). Under the same conditions in KO/KO mice, PHP.eB was unaffected whereas both 9P31 and 9P36 completely lost enhanced CNS tropism (FIG. 4C and FIG. 11B). Liver tropism, on the other hand, was decoupled from this effect, with all three viruses showing strong transduction in KO/KO mice. It is possible that any differences in the 9P31 and 9P36 potency in the periphery of KO/KO mice might be due to the pharmacokinetic effects of no longer efficient access to the CNS.

Of note, every AAV tested showed a moderate boost in infectivity in cells transfected with Slco1c1 (also known as Oatp1c1), an integral membrane anionic transporter (FIG. 3D). Unlike cells transfected with other potential receptors, however, the mNeonGreen signal in cells transfected with Slco1c1 was weak and diffuse, extending beyond cell boundaries (FIG. 9C), suggesting a transgene export or cell health phenotype. The universality of this effect and the specificity of Slco1c1 expression in the brain suggested a possible role in the weak BBB transcytosis of parent AAV9. None of the other 36 candidate receptors produced a meaningful infectivity boost for any of the 16 engineered AAVs screened.

### Example 3

### Receptor-Binding Peptides and Engineered AAVs for BBB Crossing

This example demonstrates the design of receptor-binding peptides and engineered AAVs comprising the receptor-binding peptides and illustrates their enhanced potency in BBB crossing.

### Directed Evolution of an Improved Ly6c1-Dependent Capsid

Directed evolution is a powerful method for generating biomolecules with enhanced fitness for desired properties despite an incomplete understanding of the underlying biological systems. Importantly, the outcomes of directed evolution libraries could in turn be used to unlock new biomolecules by probing the mechanism of action for molecules with evolved properties. This paradigm of reverse-engineering directed evolution has been applied to the accumulating wealth of data obtained from applying selective pressure on AAV libraries for CNS enrichment after systemic administration.

A proof-of-concept for receptor-targeted directed evolution was demonstrated. For this purpose, the murine receptor Ly6c1 (vs. the human Car4) was chosen due to the prompt availability of (1) strains with clear BBB differences, e.g. Ly6a, and (2) diverse Cre-transgenic animals for M-CREATE selections that were not available for Car4 in other species. In addition, given the mixed backgrounds of preclinical animal models, it remained important to have mechanistically distinct gene delivery vectors for rodents as well. Therefore, a single optimized capsid was engineered for broad adoption as a research tool in GPI-disrupted Ly6a mouse strains, a still unmet need, (FIG. 5A-FIG. 5D) using the previously developed M-CREATE method for AAV capsid directed evolution. This method used Cre-dependent AAV genome recovery from desired tissues and cell types after in vivo selection in Cre-transgenic mice and allowed deep characterization of selected capsids across various cell types and tissues.

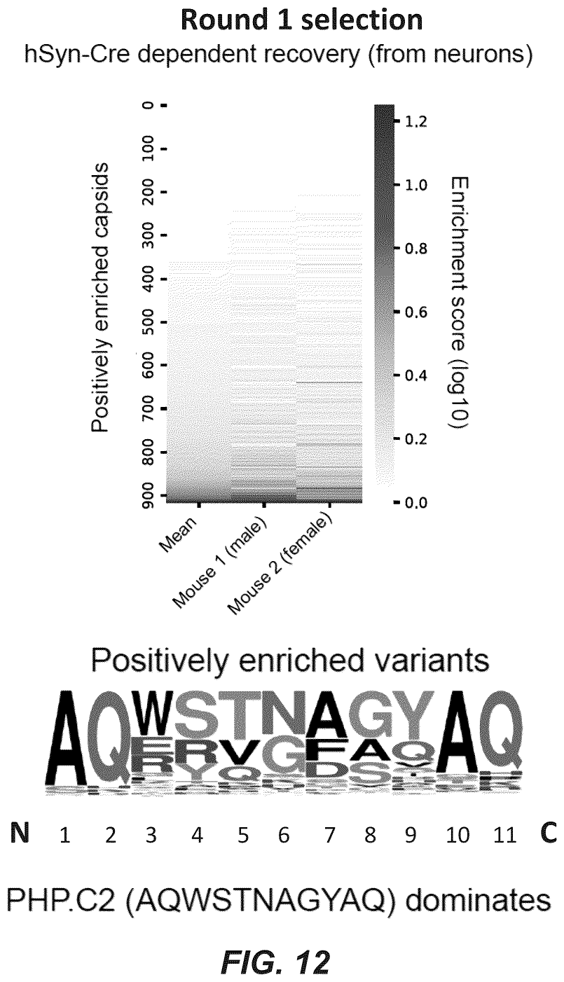

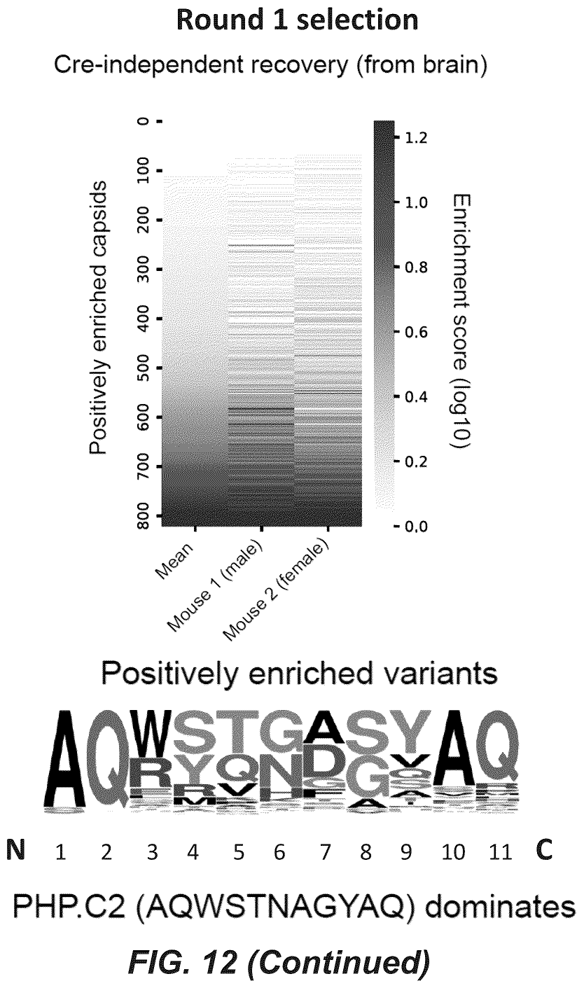

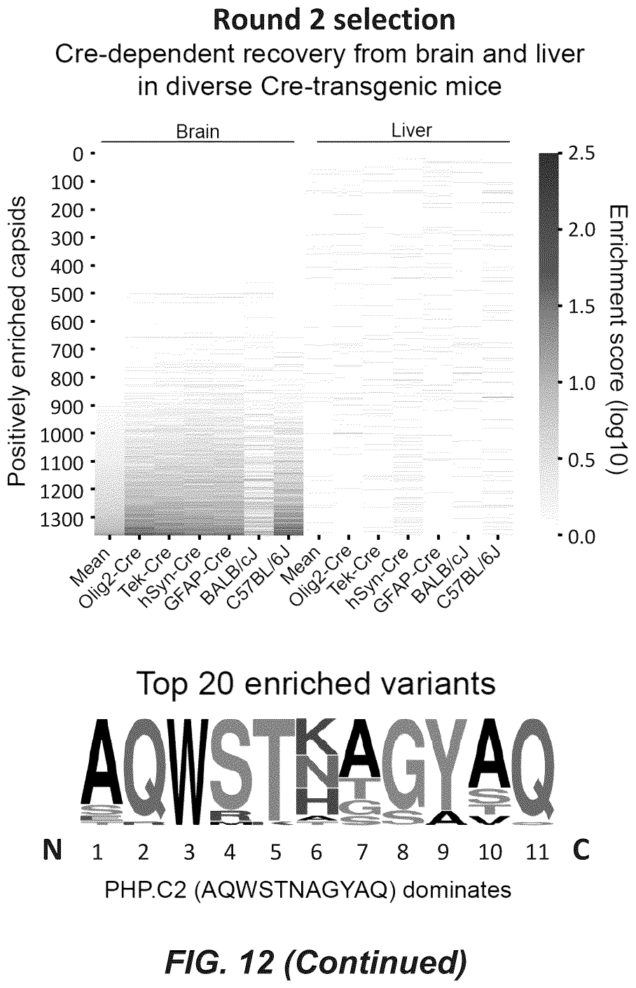

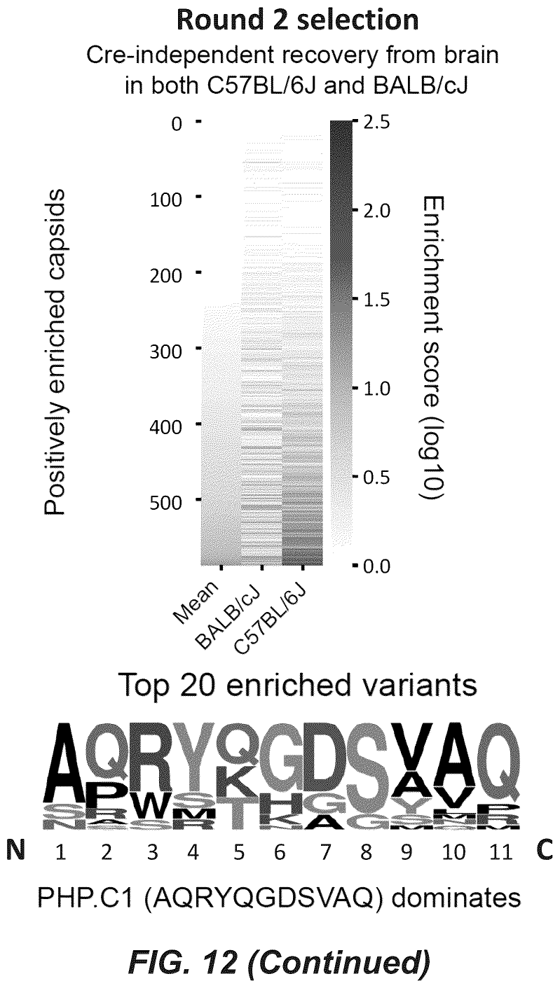

Using M-CREATE, scanning 3-mer substitution capsid libraries were constructed in the chemically-diverse Ly6c1-interacting variants PHP.C1, PHP.C2, PHP.C3, and PHP.C4. These libraries were pooled for two rounds of selection in Syn-Cre mice (FIG. 5A). In the second round of selection, Olig2-Cre, Tek-Cre, and GFAP-Cre mice, as well as wild-type C57BL/6J and BALB/cJ mice were also included so that potential differences in enhancement between these strains or cell types could be detected during selection among the variants (FIG. 5B). Interestingly, while PHP.C2 variants dominated both rounds of selection in the C57BL/6J background (Cre-dependent or not), PHP.C1 variants dominated the round 2 Cre-independent selection in BALB/cJ (FIG. 12). Following selection, top-performing variants were individually produced and characterized. AAV-PHP.eC (variant 19), evolved from PHP.C1, retained Ly6c1 interaction in cell culture (FIG. 5C) and outperformed PHP.C2 in multiple mouse strains with membrane-disrupted Ly6a (FIG. 5D). PHP.eC, thus, provided a potent tool for transgene delivery in mouse strains without membrane-localized Ly6a.

While differences in their capsid insertion sequences and in their patterns of CNS potency across genetically diverse mouse strains at low doses suggested potentially diverse mechanisms for crossing the BBB, nine capsids were found to interact with the same receptor, Ly6c1. Differences in potency across mouse strains can be highly dose-dependent, as seen with PHP.C3 and PHP.N. This suggests that, in some cases, a cell culture screening can be sensitive to interactions that might only become functionally relevant in vivo at higher doses. While several Ly6c1 polymorphisms existed across mouse strains, no clear pattern appears between mutation variants and an AAV's strain potency, similar to the observation in Ly6a-dependent AAVs. Moreover, unlike Ly6a, none of the sequence polymorphisms were predicted to interfere with Ly6c1 GPI anchoring and thus membrane localization. Ly6c1 expression levels were also consistently high across in-bred mouse strains but were significantly lower in recently wild-derived mouse strains. This suggests that low-dose potency patterns observed here may arise instead from differences in how each AAV's individual Ly6c1 binding footprint is affected by strain-dependent sequence polymorphisms, or differences in unidentified off-target interactions.

### AlphaFold2-Based Methods to Identify Receptor-Binding Peptides and Engineered AAV Binding Poses in Silico

Having identified a panel of receptor and AAV capsid pairings, binding poses for engineered AAVs and their newly identified receptors were generated using protein structure prediction methods. An AlphaFold2-based computational method for APPRAISE-AAV was applied. The APPRAISE-AAV in silico method can be applied to any existing engineered capsid library dataset to mine for capsid variants likely to interact with a chosen target receptor, including carbonic anhydrase IV (e.g., Car4). The modeling pipeline can also provide high-confidence binding models for AAV receptor complexes that have proven difficult to structurally resolve. AAVs are used as examples to demonstrate this in silico approach, however, the APPRAISE methodology is not limited to AAVs. Moreover, the pipeline for generating full AAV trimer complex structures can readily be employed to guide the translation of engineered peptide insertions identified through directed evolution in AAVs to other protein modalities

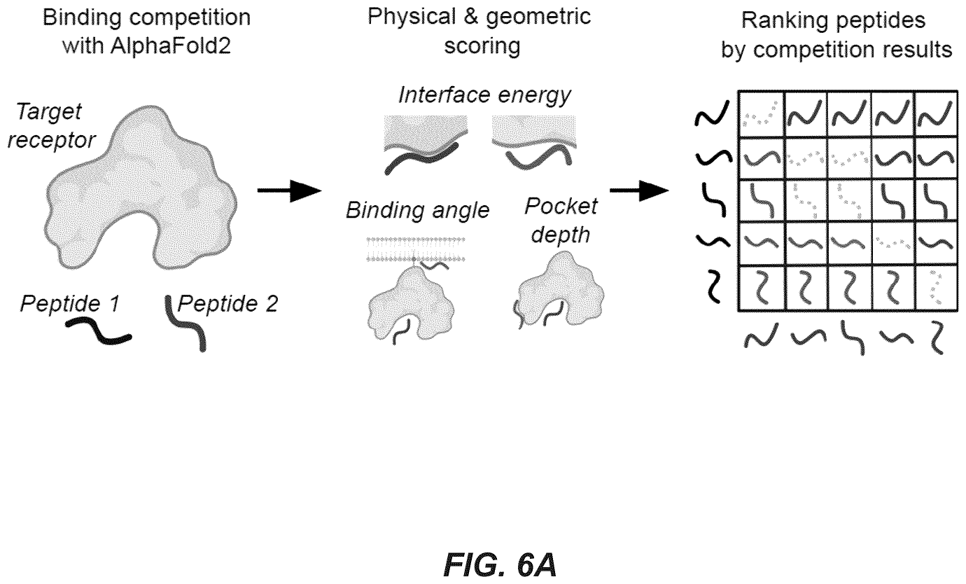

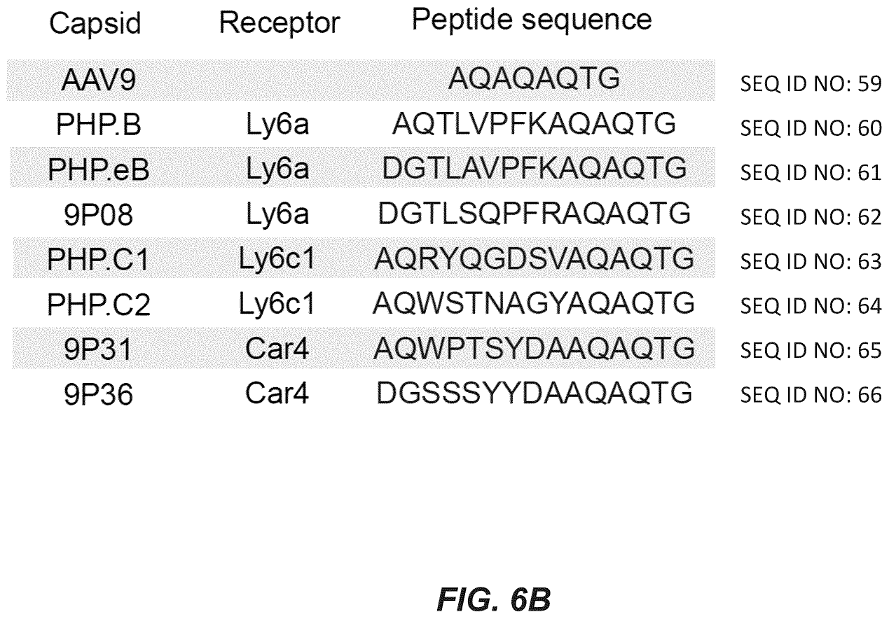

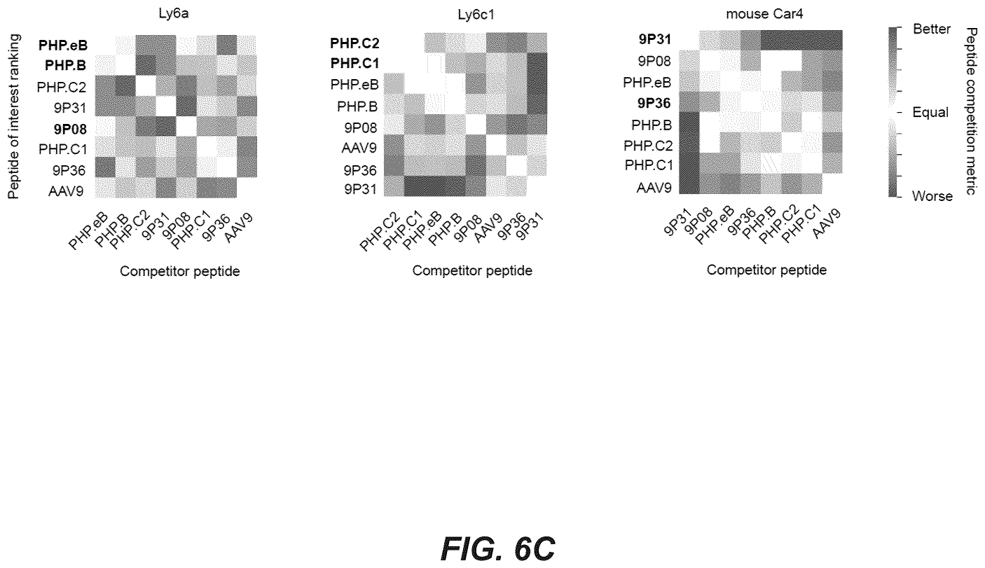

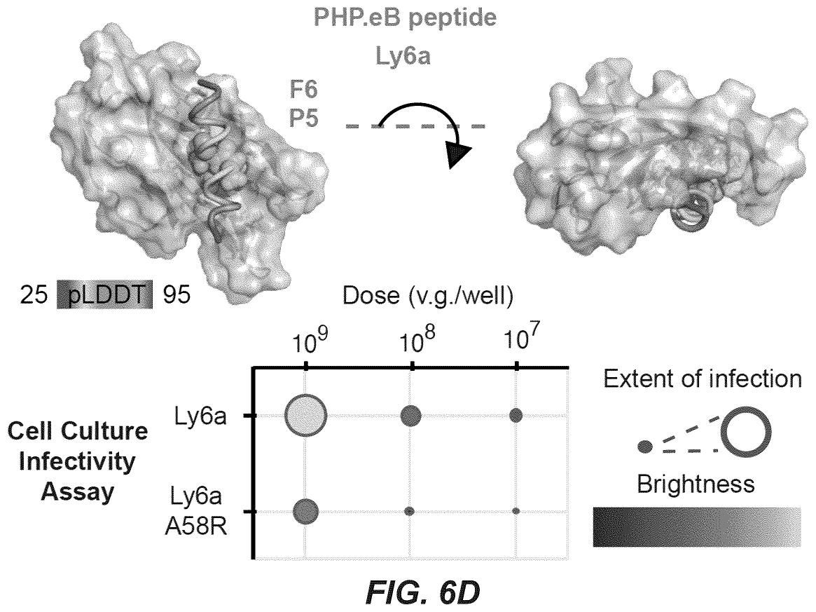

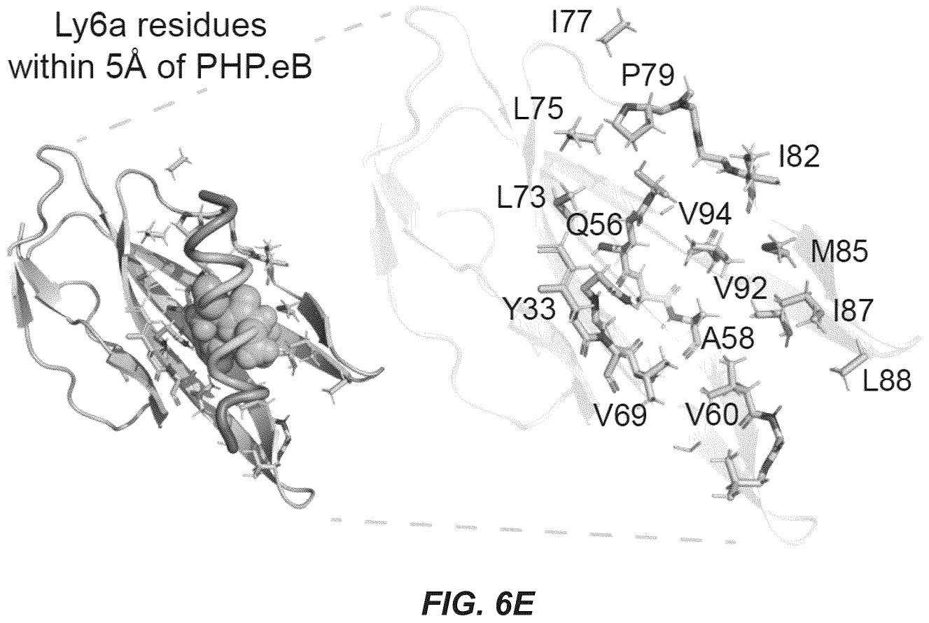

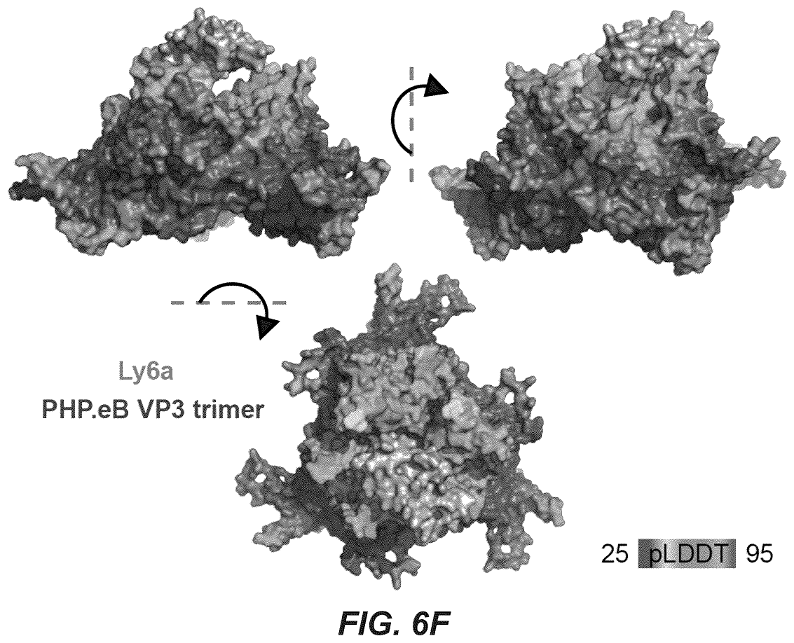

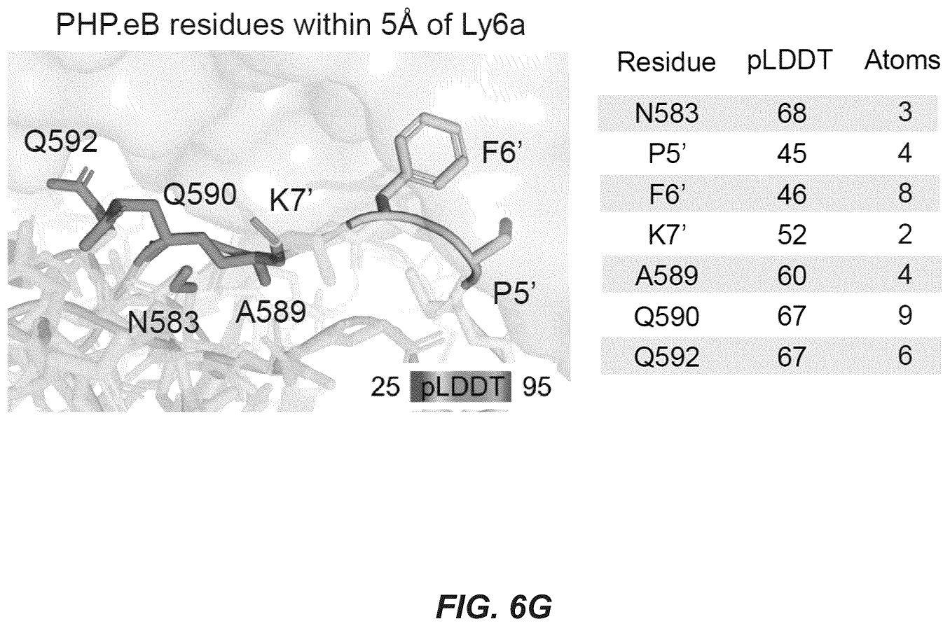

Specifically, the APPRAISE-AAV in silico method used AlphaFold2 to place surface-exposed peptides spanning mutagenic insertions (AA587-594) from two distinct AAV variants in competition to interact with a potential receptor (FIG. 6A). This comprised the minimal peptide to encompass the solvent-exposed residues of capsid variable region VIII. A combination of physical and geometric scoring parameters that included interface energy, binding angle, and binding pocket depth calculation were used to generate a peptide competition metric. Results from these individual pairwise competitions can be assembled into larger matrices that rank sets of AAV capsid insertion peptides according to their receptor-binding probability encoded in the AlphaFold2 neural network. When applied to Ly6a and the newly identified receptors, the experimentally verified Ly6a, Ly6c1, and Car4 insertion peptides raised to the top of their respective rankings (FIG. 6B-FIG. 6C). However, some false negatives were also observed, such as in 9P08 with Ly6a or 9P36 with Car4.

In addition to predictions of whether a peptide binds to a receptor, the structural details of the binding interactions were also computationally interrogated. Binding poses were generated by pairing the top AAV insertion peptide with its receptor. Each pairing was validated by repeating the cell culture screening with receptors containing point mutations hypothesized to disrupt the high-confidence region of the binding interface (as determined by the per-residue estimated model confidence pLDDT score and consistency between replicate models) in these predicted poses. The PHP.eB peptide was predicted to nestle in a groove in Ly6a, forming strong interactions at Pro5′ and Phe6′ (FIG. 6D) with several Ly6a residues (FIG. 6E). Therefore, a point mutation was introduced in this groove, Ly6a Ala58Arg, and was found to disrupt PHP.eB's enhanced infectivity with the wild-type receptor. This experimental result further bolstered confidence in in silico APPRAISE-AAV rankings.

To gain a full picture of the AAV-receptor interaction, the PHP.eB insertion peptide and Ly6a receptor complex were modeled within the context of the AAV capsid three-fold symmetry spike. This structure was challenging for standard modeling tools because of the large size of an AAV capsid (˜200 kDa per trimer) as well as the often weak and dynamic binding interactions between engineered capsids and receptors (μM affinities possible without avidity). AlphaFold2 failed to capture direct contact between full-length PHP.eB capsid and Ly6a in either a monomer-receptor or trimer-receptor configuration (data not shown). To address this challenge, an integrative structure modeling pipeline was developed. In this pipeline, an initial model of an AAV capsid trimer predicted using AlphaFold2-Multimer was structurally aligned with an AlphaFold2-predicted peptide-receptor complex model through the high-confidence Pro5′ and Phe6′ residues of the peptide insertion and RosettaRemodel optimization of the linking peptide residues within the context of the AAV capsid three-fold symmetry spike (FIG. 13A). This complete binding model (FIG. 6E), provided a snapshot for a dynamic interaction that has thus far proven resistant to high-resolution structural characterization.

The PHP.eB-Ly6a model results were consistent with available experimental results. The root-mean-square deviation (RMSD) between the PHP.eB monomer model and a cryo-EM-based model (PDB ID: 7WQO) was 0.36 angstrom. RMSD increased in PHP.eB's engineered loop to 1.36 angstroms. The only high-confidence deviation from cryo-EM structures of un-complexed PHP.eB was the side chain of Phe6′, which showed no significant electron density, indicating flexibility, but formed a stable interaction with Ly6a in the PHP.eB monomer model (FIG. 13B, right). The high confidence prediction of Pro5′ and Phe6′ aligned with recent evidence showing that PFK 3-mer insertion alone was sufficient to gain Ly6a binding. While Ly6a can bind any insertion loop of a trimer, additional interactions induced steric clashes supporting a ratio of one Ly6a per capsid trimer. Interestingly, a PHP.eB-Ly6a complex ensemble image forced to contain 60 bound copies of Ly6a resembled a recently reported CryoEM map, whose analysis pipeline would average all 60 singly-occupied binding sites to form a composite map (FIG. 13C). The PHP.eB-Ly6a complex model showed that a single copy of both Ly6a and AAVR PKD2 domain can bind to the same three-fold spike simultaneously without clashing (FIG. 13D), in agreement with saturation binding experiments. Consistent with previous work showing that the Ly6a SNP D63G did not affect PHP.eB binding, the residue was greater than 10 angstroms from the PHP.eB peptide atoms in the PHP.eB-Ly6a complex model. The PHP.eB-Ly6a complex model included several interactions involving AAV insertion-adjacent residues, which was consistent with a previous report (FIG. 6G).

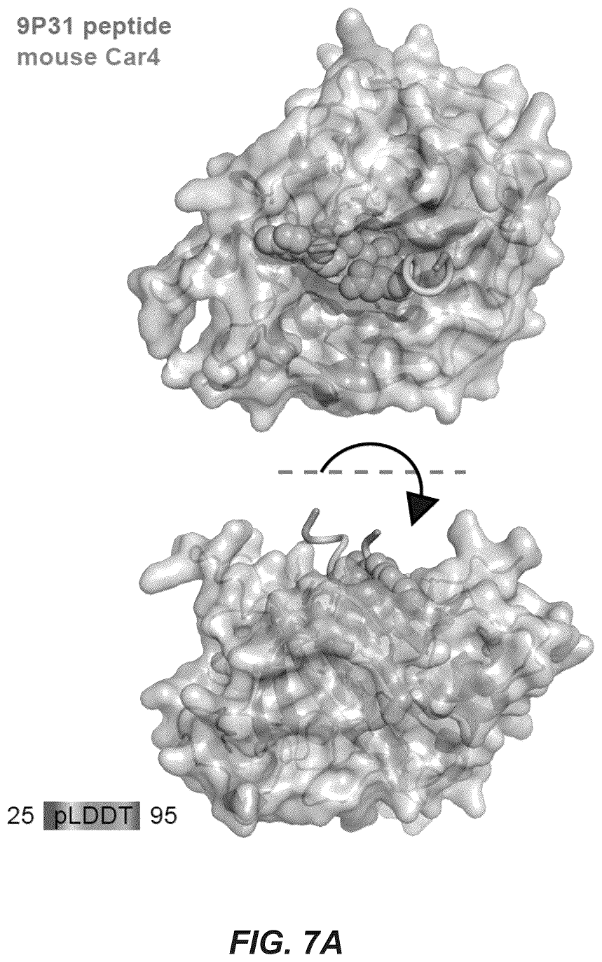

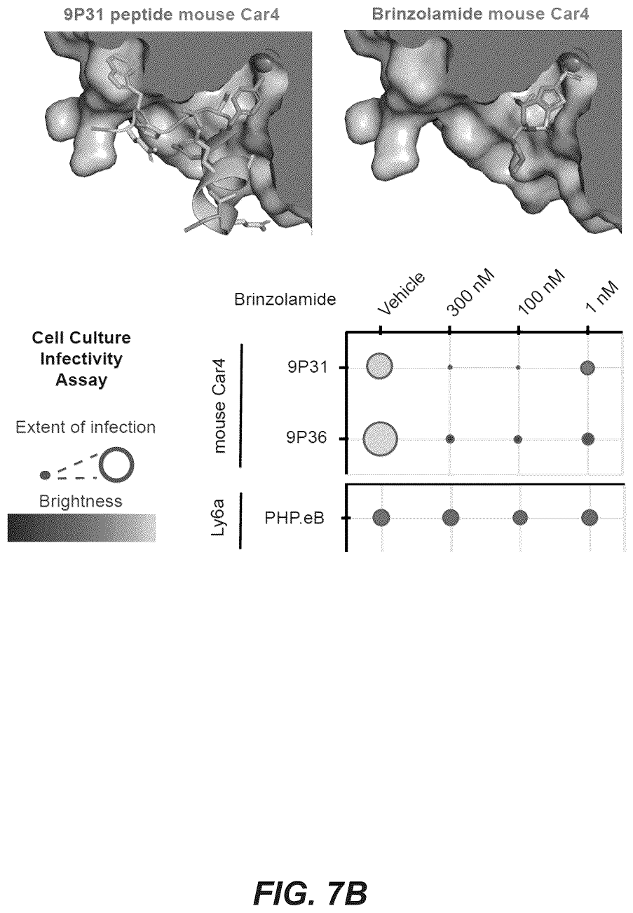

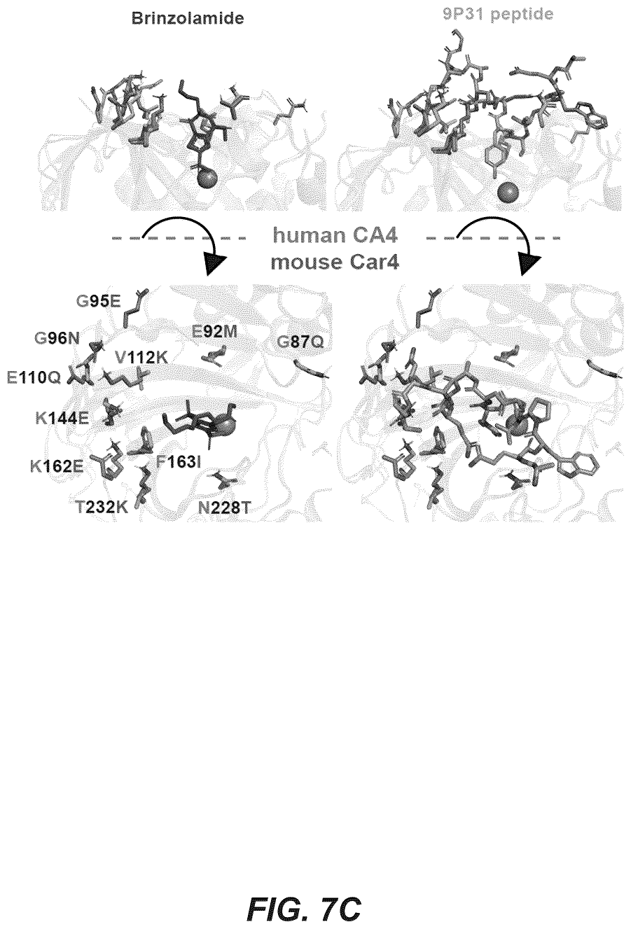

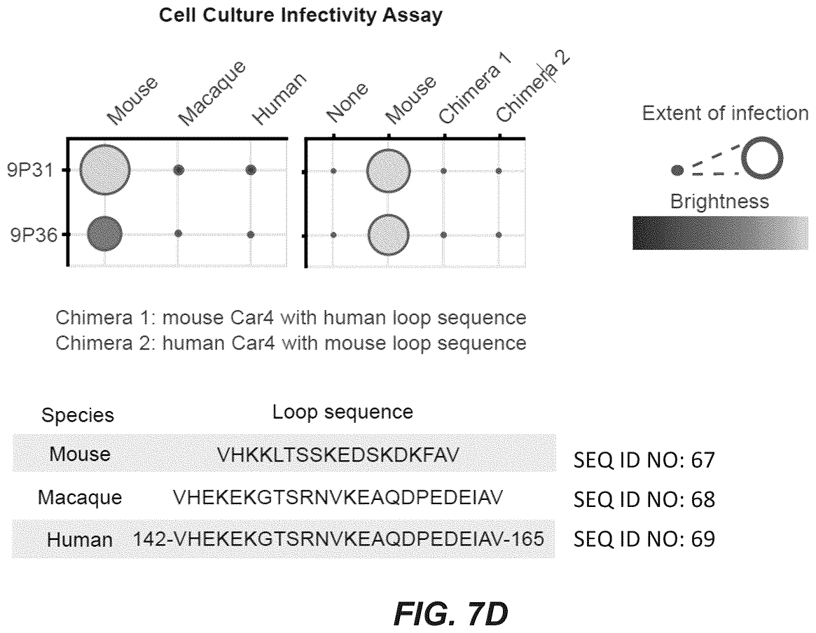

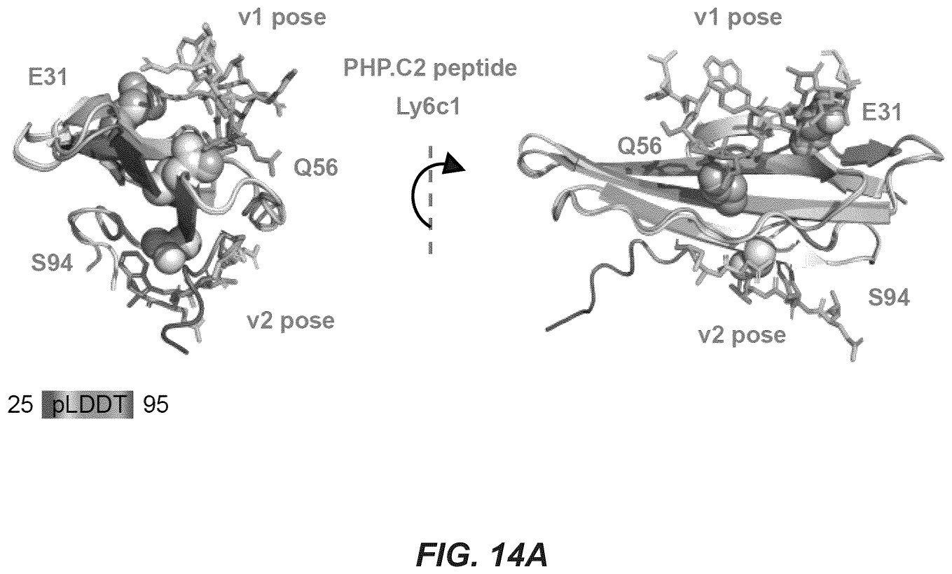

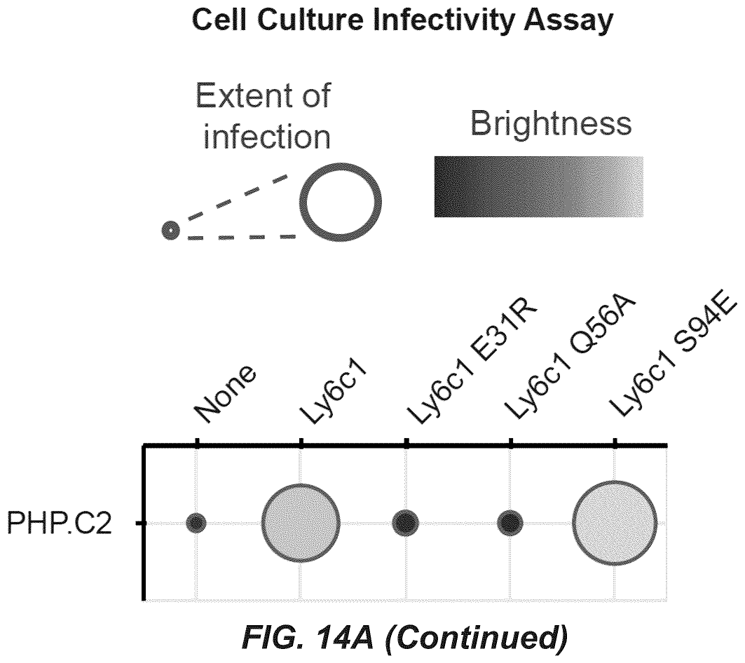

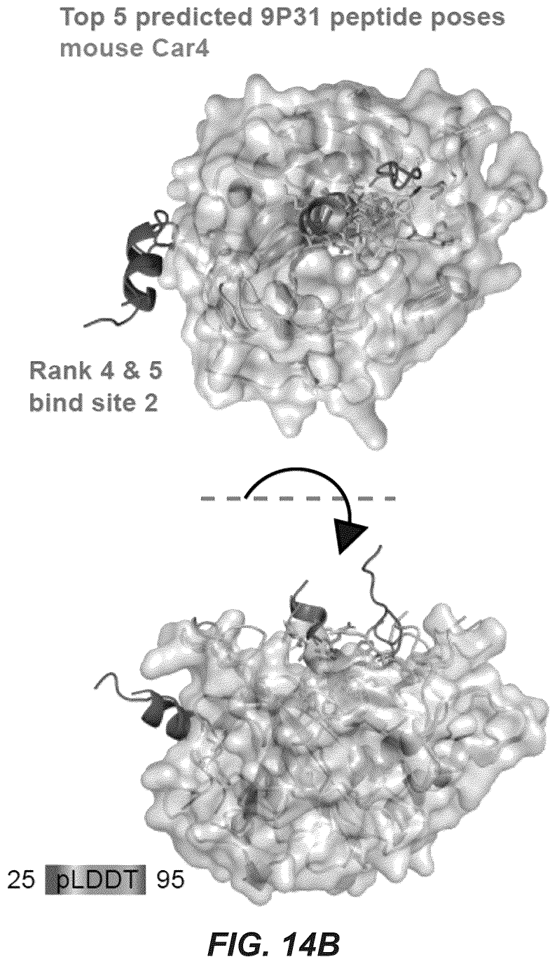

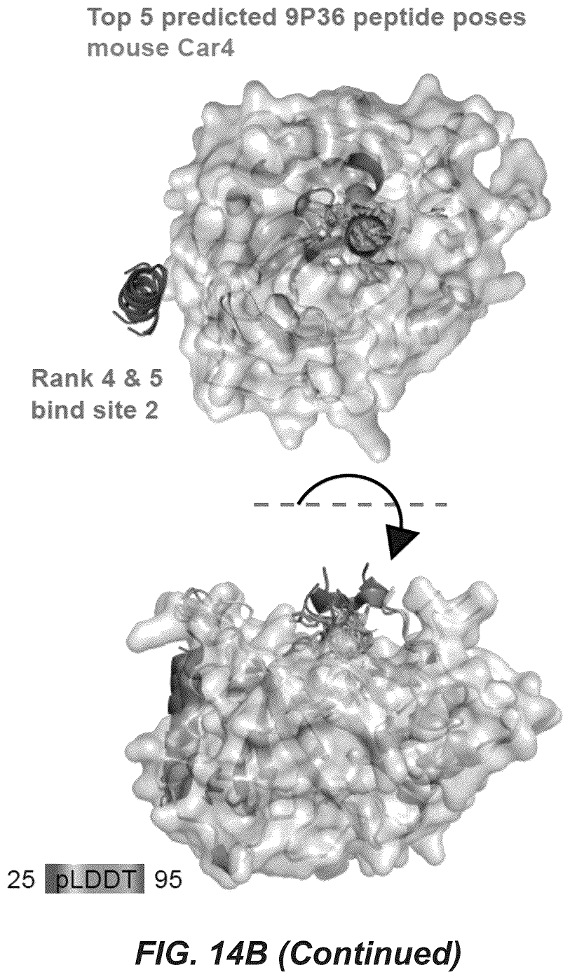

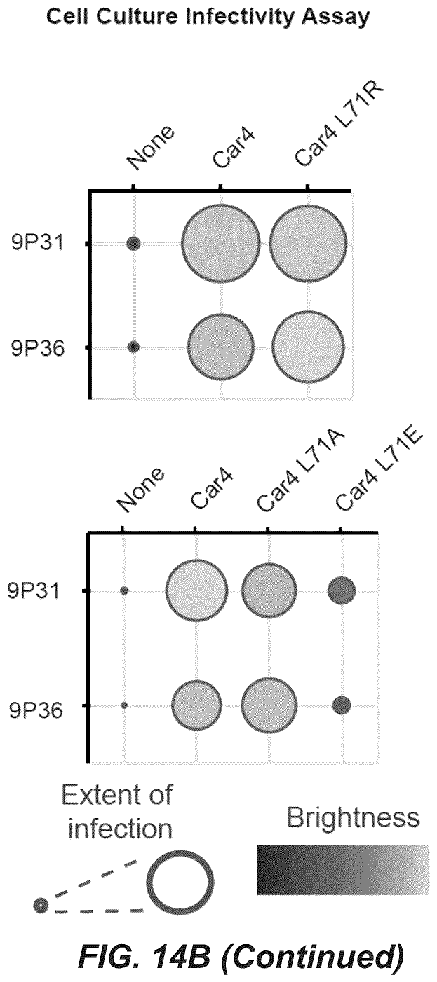

These structural modeling methods were then applied to the newly identified receptors. Interestingly, unlike for Ly6a and Car4, the predicted binding pose for PHP.C2 peptide with Ly6c1 was found to vary with the version of AlphaFold-Multimer used, with v1 predictions closely matching mutational data from the cell culture infectivity assay (FIG. 14A). Such complementarity between versions has been reported previously. In mouse Car4, 9P31 peptide invaded the catalytic pocket of the enzyme (FIG. 7A). The 9P31 tyrosine residue shared with 9P36 approaches the enzyme active site and 9P31's divergent tryptophan finds purchase in an ancillary pocket (FIG. 7B). This predicted binding pose was competitive with the binding site of brinzolamide (PDB ID 3NZC), a broad carbonic anhydrase inhibitor that is prescribed for glaucoma. In the cell culture infectivity assay, brinzolamide showed a dose-dependent inhibition of 9P31 and 9P36 potency, while PHP.eB was unaffected (FIG. 7B). The smaller brinzolamide bound deep in the catalytic core of Car4 where side chains were largely conserved between species (FIG. 7C). However, 9P31 peptide extended to the surface of the enzyme where there was considerable sequence divergence. Similarly, while brinzolamide bound to both mouse and human Car4, 9P31 and 9P36 were specific to mouse Car4 (FIG. 7D). Chimeric receptors that swapped a highly divergent loop of the 9P31 binding site showed that this region was necessary but not sufficient to control 9P31 and 9P36 potency. A second potential Car4 binding site was also suggested by lower ranked poses with 9P31 and 9P36, but mutagenesis experiments showed inconsistent effects (FIG. 14B). Future rational engineering of new AAVs against species-appropriate CA4, aided by the APPRAISE-AAV method, is a promising new avenue for the generation of non-invasive vectors with enhanced CNS potency. Targeting CA4 can also find applications across diverse proteins and chemical modalities. In vivo experiments can be carried out to further validate the engineering of a human CA-binding AAV with optimal BBB crossing properties.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of increasing permeability of the blood brain barrier, comprising:
providing a targeting peptide capable of binding to a carbonic anhydrase IV, thereby increasing permeability of the blood brain barrier, wherein the targeting peptide binds a zinc binding site and/or hydrophobic substrate binding pocket of the carbonic anhydrase IV.

2. (canceled)

3 (canceled)

4 (canceled)

5 The method of claim 1, wherein the permeability of the blood brain barrier is increased by at least 25%, 50%, 75%, 100%, or more as compared to the absence of the target peptide or the reduction of carbonic anhydrase IV activity.

6. A method of delivering a payload to a nervous system, the method comprising:
providing a targeting peptide capable of binding to a carbonie anhydrase IV or a derivative thereof, wherein the targeting peptide is part of a delivery system, and wherein the delivery system comprises the payload to be delivered to a nervous system; and
administering the delivery system to the subject.

7. The method of claim 6, wherein the delivery system comprises nanoparticles, nanotubes, nanowires, dendrimers, liposomes, ethosomes and aquasomes, polymersomes and niosomes, foams, hydrogels, cubosomes, quantum dots, exosomes, macrophages, and combinations thereof.

8. The method of claim 6, wherein the delivery system comprises a viral vector or a non-viral vector, wherein the viral vector comprises an adenovirus vector, an AAV vector, a lentiviral vector, or a retrovirus vector.

9. The method of claim 8, wherein the target peptide is part of a capsid protein of an AAV vector.

10. The method of claim 9, wherein the AAV vector is a vector selected from the group consisting of AAV1, AAV2, AAV3, AAV3b, AAV4, AAV5, AAV6, AAV7, AAV8, AAV9, AAV10, AAV-DJ, human isolate hu.31, human isolate hu.32, rhesus isolate rh.8rhesus isolate rh. 10, and a variant thereof.

11. The method of claim 8, wherein the non-viral vector comprises lipid-based nanoparticles, polymeric nanoparticles, inorganic nanoparticles, surfactant-based emulsions, nanowires, silica nanoparticles, peptide or protein-based particles, lipid-polymer particles, nanolipoprotein particles, and combinations thereof.

12. The method of claim 6, wherein the payload to be delivered to a nervous system is a biological molecule, a non-biological molecule, or a combination thereof; and wherein the biological molecule is selected from the group consisting of a nucleic acid sequence, a protein, a peptide, a lipid, a polysaccharide, and a combination thereof.

13. The method of claim 6, wherein the payload is a therapeutic molecule.

14. The method of claim 12, wherein the nucleic acid sequence to be delivered to a nervous system comprises one or more of:
a) a trophic factor, a growth factor, or other soluble factors that might be released from the transduced cells and affect the survival or function of that cell and/or surrounding cells;
b) a cDNA that restores protein function to humans or animals harboring a genetic mutation(s) in that gene;
c) a cDNA that encodes a protein that can be used to control or alter the activity or state of a cell;
d) a cDNA that encodes a protein or a nucleic acid used for assessing the state of a cell:
c) a cDNA and/or associated guide RNA for performing genomic engineering;
f) a sequence for genome editing via homologous recombination;
g) a DNA sequence encoding a therapeutic RNA;
h) an shRNA or an artificial miRNA delivery system; or
i) a DNA sequence that influences the splicing of an endogenous gene.

15. The method of claim 6, wherein the carbonic anhydrase IV is a mouse carbonic anhydrase IV having at least 80% sequence identity to an amino acid sequence of SEQ ID NO: 179, or the carbonic anhydrase TV is a human carbonic anhydrase IV having at least 80% sequence identity to an amino acid sequence of SEQ ID NO: 180.

16 (canceled)

17. The method of claim 6, wherein upon binding the targeting peptide is capable of interacting with (1) one or more positions functionally equivalent to S20, G21, W22, L36, W41, P42, E90, V111, Q112, H114, H139, V141, K143, F156, L217, T218, T219, P220, N221, D223, or W228 in the carbonic anhydrase IV having an amino acid sequence of SEQ ID NO: 179; or (2) one or more positions functionally equivalent to S21, H22, W23, L37, W42, G43, M92, K113, Q114, H116, H141,V143, E145, Q158, L224, T225, T226, P227, T228, D231, or W236 in the carbonic anhydrase IV having an amino acid sequence of SEQ ID NO: 180.

18. The method of claim 6, wherein the targeting peptide comprises (1) an amino acid sequence selected from the group consisting of SEO ID NOs: 70-174; or (2) an amino acid sequence having at least 80% sequence identity to an amino acid sequence selected from the group consisting of SEQ ID NOs: 70-174.

19. The method of claim 6, wherein the targeting peptide comprises (1) an amino acid sequence selected from the group consisting of: AKPTPLLGLLQAQTG (SEQ ID NO: 70), AKPTPLLLLLQAQTG (SEQ ID NO: 71), AQPPLGGLLAQAQTG (SEQ ID NO: 72). AKPPGPWAEAQAQTG (SEQ ID NO: 73), and AQPPLLGGLAQAQTG (SEQ ID NO: 74); or (2) an amino acid sequence having at least 80% sequence identity to an amino acid sequence selected from the group consisting of SEQ ID NOs: 70-74.

20. The method of claim 9, wherein the targeting peptide is inserted between two adjacent amino acids in AA587-594 of SEQ ID NO: 178 of the AAV9 vector or functional equivalents of AA587-594 in an amino acid sequence at least 80% identical to SEQ ID NO: 178.

21. The method of claim 9, wherein the targeting peptide is inserted between AA588-589 of SEQ ID NO: 178 of the AAV9 vector or functional equivalents of AA588-589 in an amino acid sequence at least 80% identical to SEQ ID NO: 178.

22. The method of claim 9, wherein the AAV vector is conjugated to a nanoparticle, a second molecule, or a combination thereof.

23. The method of claim 6, wherein the administration is a systemic administration; and wherein the administration is an intravenous administration or an intrathecal administration.

24. The method of claim 6, wherein the subject is a human.

25. The method of claim 6, wherein the subject is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, or lysosomal storage disorders that involve cells within the CNS; and optionally wherein the lysosomal storage disorder that involve cells within the CNS is Krabbe disease, Sandhoff disease, Tay-Sachs, Gaucher disease (Type I, II or III), Niemann-Pick disease (NPC1 or NPC2 deficiency). Hurler syndrome, Pompe Disease, or Batten disease.

26. The method of claim 6, wherein the subject is a subject suffering from, at risk to develop, or has suffered from a stroke, traumatic brain injury, epilepsy, or spinal cord injury.

27.-55. (anceled)

56. A method of increasing permeability of the blood brain barrier in a murine species, comprising: providing a targeting peptide capable of binding to Ly6c1, thereby increasing permeability of the blood brain barrier in the murine species.

57. The method of claim 56, wherein the targeting peptide has 1) an amino acid sequence selected from the group consisting of AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), and SPRYKGDSVAQ (SEQ ID NO: 57); or (2) an amino acid sequence having at least 80% sequence identity to an amino acid sequence selected from the group consisting of AQRYQGDSVAQ (SEQ ID NO: 7), AQWSTNAGYAQ (SEQ ID NO: 8), AQERVGFAQAQ (SEQ ID NO: 9), AQWMTHGSAAQ (SEQ ID NO: 10), and SPRYKGDSVAQ (SEQ ID NO: 57).

58. (canceled)

59. The method of claim 57, wherein the targeting peptide is inserted between two adjacent amino acids in AA587-590 of SEQ ID NO: 178 or functional equivalents of AA587-590 in an amino acid sequence at least 80% identical to SEQ ID NO: 178.
